(19) United States
(12) Patent Application Publication
(10) Pub. No.: US 2026/0355335 A1
(43) Pub. Date: Nov. 12, 2026

(54) COMPOSITIONS AND USES THEREOF

(71) Applicant: Solve Therapeutics, Inc., San Diego, CA (US)

(72) Inventors: Brian LANNUTTI, Solana Beach, CA (US); Jeff WATKINS, Encinitas, CA (US); Katti JESSEN, San Diego, CA (US)

(21) Appl. No.: 19/115,167

(22) PCT Filed: Sep. 28, 2023
(86) PCT No.: PCT/US2023/075435

§ 371 Date: Mar. 25, 2025

Related U.S. Application Data
(60) Provisional application No. 63/377,480, filed on Sep. 28, 2022.
(60) Provisional application No. 63/377,612, filed on Sep. 29, 2022.

(51) Int. Cl. A61K 47/68; A61P 35/00

(52) U.S. Cl. CPC A61K 47/68037; A61K 47/6849; A61K 47/6871; A61P 35/00

(57) ABSTRACT

Provided herein is a compound of Formula (I): or a pharmaceutically acceptable salt thereof, wherein the variables of Formula (I) are as defined in the application. Such compounds can be useful as anti-cancer agents.

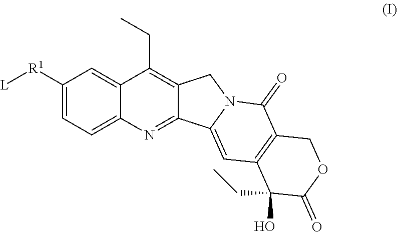

### CROSS-REFERENCE TO RELATED APPLICATIONS

This application claims the benefit of U.S. Provisional Patent Application No. 63/377,480, filed on Sep. 28, 2022; and U.S. Provisional Patent Application No. 63/377,612, filed on Sep. 29, 2022, each of which is hereby incorporated by reference in its entirety.

### BACKGROUND

Currently, small cytotoxic molecules for antibody drug conjugates can include camptothecin derivatives, which have antitumor effects by inhibiting topoisomerase I.

Camptothecin derivatives can be used in antibody drug conjugates (ADC). However, there is still a need for further development of camptothecin derivatives and ADC drugs with better efficacy and/or safety.

### SUMMARY OF THE INVENTION

In an aspect, the present disclosure provides a compound represented by Formula (I),

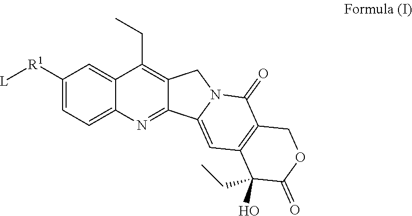

or a pharmaceutically acceptable salt thereof, wherein;
R¹ is selected from —O—CH₂—CH₂—O— and —NH—C(O)—CH₂—O—;
L is L²-(L²B)_{z}-L³-L⁴-(L⁵)ₘ-(L⁶)ₙ-(L⁷)ₚ-R²;
L² is selected from a C₁₋₆ alkylene;
L^{2B} is selected from NR⁴C(O)O—C₁₋₆ alkylene-phenylene, wherein the phenylene is optionally substituted with one or more R⁵;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocyclene;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
R⁴ is selected from hydrogen and C₁₋₆ alkyl optionally substituted with one or more SO₂C₁₋₆ alkyl;
each R⁵ is independently selected from a sugar;
m is selected from 0 and 1;
n is selected from 0 and 1;
p is selected from 0 and 1;
q is selected from 0 to 8;
s is selected from 0 and 1; and
z is selected from 0 and 1.

In an aspect, the present disclosure provides a compound represented by Formula (I)*,

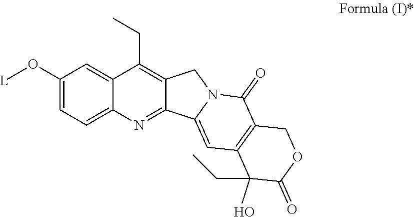

or a pharmaceutically acceptable salt thereof, wherein;
   L is L²-L³-L⁴-(L⁵)ₘ-(L⁶)ₙ-(L⁷)ₚ-R²;
   L² is selected from a C₁₋₆ alkylene;
   L³ is selected from a residue comprising 1 to 7 amino acids;
   L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
   L⁵ is selected from an (O—CH₂—CH₂—)_{q}—(NR³)ₛ;
   L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   L⁷ is selected from a C₅₋₆ carbocyclene;
   R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   R³ is selected from hydrogen and C₁₋₆ alkyl;
   m is selected from 0 and 1;
n is selected from 0 and 1;
p is selected from 0 and 1;
q is selected from 0 to 8; and
s is selected from 0 and 1.

In some embodiments, Formula (I) is represented by

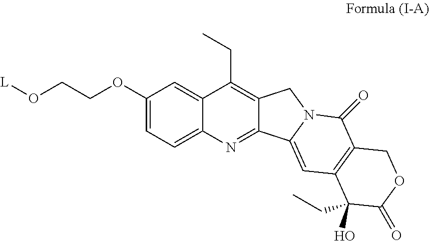

or a pharmaceutically acceptable salt thereof.

In some embodiments, Formula (I) is represented by

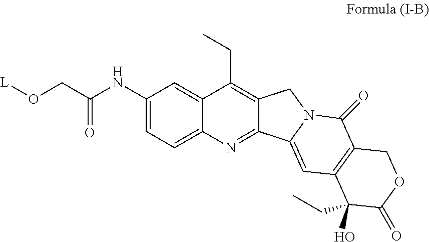

or a pharmaceutically acceptable salt thereof.

In some embodiments, Formula (I) is represented by

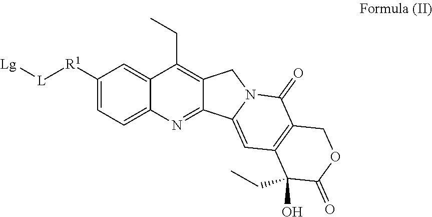

or a pharmaceutically acceptable salt thereof, wherein;
Lg is the ligand.

In some embodiments, the present disclosure provides a compound of Formula (III):

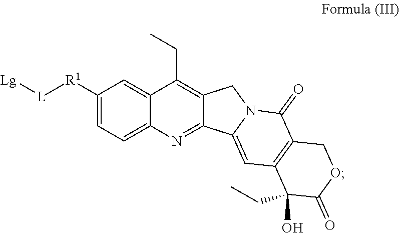

or a pharmaceutically acceptable salt thereof, wherein;
Lg is a ligand;
R¹ is selected from —O—CH₂—CH₂—O— and —NH—C(O)—CH₂—O—;
L is L²-L³-L⁴-(L⁵)ₘ-(L⁶)ₙ-(L⁷)ₚ-R²;
L² is selected from a C₁₋₆ alkylene;
L³ is selected from a residue comprising 1 to 7 amino acids;
   L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an —(O—CH₂—CH₂—)_{q}—(NR³)ₛ—;
   L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   L⁷ is selected from a C₅₋₆ carbocyclene;
   R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   R³ is selected from hydrogen and C₁₋₆ alkyl;
m is selected from 0 and 1;
n is selected from 0 and 1;
p is selected from 0 and 1;
q is selected from 0 to 8; and
s is selected from 0 and 1.

In some embodiments, Formula (I)* is represented by

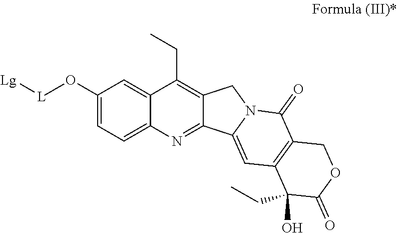

or a pharmaceutically acceptable salt thereof, wherein;
Lg is the ligand.

In some embodiments, the present disclosure provides a compound of Formula (IV)*:

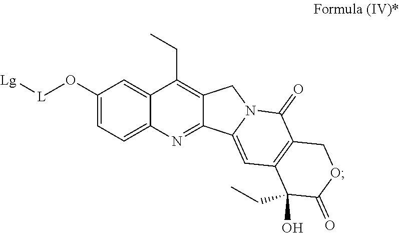

or a pharmaceutically acceptable salt thereof, wherein;
Lg is a ligand;
L is L²-L³-L⁴-(L⁵)ₘ-(L⁶)ₙ-(L⁷)ₚ-R²;
L² is selected from a C₁₋₆ alkylene;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocyclene;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
m is selected from 0 and 1;
n is selected from 0 and 1;
p is selected from 0 and 1;
q is selected from 0 to 8; and
s is selected from 0 and 1.

In some embodiments, the ligand is selected from an antibody or an antigen-binding fragment thereof. In some cases, the ligand is selected from a group consisting of chimeric antibodies, humanized antibodies, and fully human antibodies.

In some embodiments, a pharmaceutical composition comprising a compound or salt of Formula (I), Formula (I)*, Formula (I-A), Formula (I-B), Formula (II), Formula (II)*, Formula (III), Formula (III)*, Formula (IV), Formula (IV)*, Formula (IV-A)*, Formula (V-A), Formula (V-B), Formula (V-C), or Formula (V-D), and a pharmaceutically acceptable excipient of any one thereof.

In some embodiments, the disclosure provides a method of treating a subject with a tumor, comprising administering to the subject in need thereof a compound or salt of Formula (I), Formula (I)*, Formula (I-A), Formula (I-B), Formula (II), Formula (II)*, Formula (III), Formula (III)*, Formula (IV), Formula (IV)*, Formula (IV-A)*, Formula (V-A), Formula (V-B), Formula (V-C), or Formula (V-D), or a pharmaceutical composition of any one thereof.

In some embodiments, the disclosure provides a method of treating a subject with a disease or disorder, comprising administering to the subject in need thereof a compound or salt of Formula (I), Formula (I)*, Formula (I-A), Formula (I-B), Formula (II), Formula (II)*, Formula (III), Formula (III)*, Formula (IV), Formula (IV)*, Formula (IV-A)*, Formula (V-A), Formula (V-B), Formula (V-C), or Formula (V-D), or a pharmaceutical composition of any one thereof.

In some embodiments, the disclosure provides a method of treating a subject with a cancer, comprising administering to the subject in need thereof a compound or salt of Formula (I), Formula (I)*, Formula (I-A), Formula (I-B), Formula (II), Formula (II)*, Formula (III), Formula (III)*, Formula (IV), Formula (IV)*, Formula (IV-A)*, Formula (V-A), Formula (V-B), Formula (V-C), or Formula (V-D), or a pharmaceutical composition of any one thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates the stability of ADCs in mouse plasma;

FIG. 2 illustrates the stability of ADCs in human plasma;

FIG. 3 illustrates in vivo antitumor activity of ADCs in the Jeko-1 xenograft model; and

FIG. 4 illustrates in vivo antitumor activity of ADC-3 in the SK-OV3 xenograft model.

### DETAILED DESCRIPTION OF THE INVENTION

The following description sets forth numerous exemplary configurations, methods, parameters, and the like. It should be recognized, however, that such description is not intended as a limitation on the scope of the present disclosure, but is instead provided as a description of exemplary embodiments.

In the following description, certain specific details are set forth in order to provide a thorough understanding of various embodiments of the disclosure. However, one skilled in the art will understand that the disclosure may be practiced without these details.

### DEFINITIONS

Unless defined otherwise, all technical and scientific terms used herein have the same meaning as is commonly understood by one of skill in the art to which this invention belongs. All patents and publications referred to herein are incorporated by reference.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{c} is an alkynylene chain as defined above.

“Carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Bicyclic carbocycles may be fused, bridged or spiro-ring systems. In some cases, spiro-ring carbocycles have at least two molecular rings with only one common atom.

“Carbocyclene” refers to a divalent carbocycle linking the rest of the molecule to a radical group.

The term “unsaturated carbocycle” refers to carbocycles with at least one degree of unsaturation and excluding aromatic carbocycles. Examples of unsaturated carbocycles include cyclohexadiene, cyclohexene, and cyclopentene.

“Cycloalkyl” refers to a fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Aminoalkyl” refers to an alkyl radical, as defined above, that is substituted by one or more amine radicals, for example, propan-2-amine, butane-1,2-diamine, pentane-1,2,4-triamine and the like.

“Hydroxyalkyl” refers to an alkyl radical, as defined above, that is substituted by one or more hydroxy radicals, for example, propan-1-ol, butane-1,4-diol, pentane-1,2,4-triol, and the like.

“Alkoxyalkyl” refers to an alkyl radical, as defined above, that is substituted by one or more alkoxy radicals, for example, methoxymethane, 1,3-dimethoxybutane, 1-methoxypropane, 2-ethoxypentane, and the like.

“Cyanoalkyl” as used herein refers to an alkyl radical, as defined above, that is substituted by one or more cyano radicals, for example, acetonitrile, 2-ethyl-3-methylsuccinonitrile, butyronitrile, and the like.

“Heterocycle” refers to a saturated or unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. Bicyclic heterocycles may be fused, bridged or spiro-ring systems. In some cases, spiro-ring heterocycles have at least two molecular rings with only one common atom. The spiro-ring heterocycle includes at least one heteroatom.

“Heterocyclene” refers to a divalent heterocycle linking the rest of the molecule to a radical group.

“Heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, pyran, thiophene, isoxazole, benzimidazole, benzthiazole, and imidazopyridine.

An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine. Heterocycles may be optionally substituted by one or more substituents such as those substituents described herein.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R b-N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{C} is a straight or branched alkylene, alkenylene or alkynylene chain.

As used in the specification and claims, the singular form “a” “an” and “the” includes plural references unless the context clearly dictates otherwise.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “ligand” generally refers to a macromolecular compound capable of recognizing and binding to an antigen or receptor associated with a target cell. The ligand can be used to bring the drug to the target cell population that binds to the ligand, including but not limited to protein hormones, lectins, growth factors, antibodies, or others that can bind to cells, receptors and/or antigens molecule. The ligand can be an antibody. The ligand can be an antigen binding fragment. The ligand can be a targeting moiety.

The term “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target biological molecule may be a biological receptor or other structure of a cell such as a tumor antigen.

The term “linker” refers to a chemical moiety that is capable of combining two different chemical moieties to each other. The linker can include spacers and amino acids. The linker can be a cleavable linker that facilitates the release of the compounds described herein. The linker can be used to form a covalent bond to a ligand (e.g., antibody).

The term “antibody” means whole antibodies and any antigen binding fragment (i.e., “antigen-binding portion”) or single chain variants thereof. A whole antibody is a protein comprising at least two heavy (H) chains and two light (L) chains inter-connected by disulfide bonds. Each heavy chain comprises a heavy chain variable region (VH) and a heavy chain constant region comprising three domains, CH1, CH2 and CH3. Each light chain comprises a light chain variable region (VL or Vk) and a light chain constant region comprising one single domain, CL. The VH and VL regions can be further subdivided into regions of hypervariability, termed complementarity determining regions (CDRs), interspersed with more conserved framework regions (FRs). Each VH and VL comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (C1q) of the classical complement system.

An antibody is said to “specifically bind” to an antigen X if the antibody binds to antigen X with a KD of 5×10₋₈ M or less, more preferably 1×10⁻⁸ M or less, more preferably 6×10⁻⁹ M or less, more preferably 3×10⁻⁹M or less, even more preferably 2×10⁻⁹M or less. The antibody can be chimeric, humanized, or, preferably, human. The heavy chain constant region can be engineered to affect glycosylation type or extent, to extend antibody half-life, to enhance or reduce inter-actions with effector cells or the complement system, or to modulate some other property. The engineering can be accomplished by replacement, addition, or deletion of one or more amino acids or by replacement of a domain with a domain from another immunoglobulin type, or a combination of the foregoing.

The term “antigen binding fragment” and “antigen binding portion” of an antibody (or simply “antibody portion” or “antibody fragment”) mean one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, for example, Abbas et al., Cellular and Molecular Immunology, 6th Ed., Saunders Elsevier 2007); (iv) a Fd fragment consisting of the VH and CH1 domains; (v) a Fv fragment consisting of the VL and VH domains of a single arm of an antibody, (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains. Preferred antigen binding fragments are Fab, F(ab′)2, Fab′, Fv, and Fd fragments. Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” of an antibody.

The term “isolated antibody” means an antibody that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

The term “monoclonal antibody” or “monoclonal antibody composition” means a preparation of antibody molecules of single molecular composition, which displays a single binding specificity and affinity for a particular epitope.

The term “human antibody” means an antibody having variable regions in which both the framework and CDR regions (and the constant region, if present) are derived from human germline immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human antibody” does not include antibodies in which CDR sequences derived from the germline of another mammalian species, such as a mouse, have been grafted onto human framework sequences.

The term “human monoclonal antibody” means an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

### Compounds of the Disclosure

The following is a discussion of compounds and salts thereof that can be used in the methods of the disclosure.

In an aspect, the present disclosure provides a compound of Formula (I):

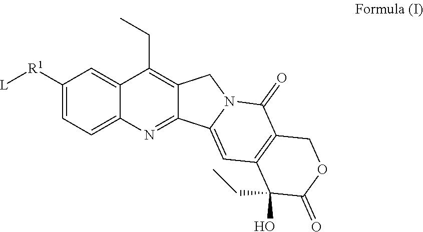

or a pharmaceutically acceptable salt thereof, wherein;
R¹ is selected from —O—CH₂—CH₂—O— and —NH—C(O)—CH₂—O—; L is L²-(L^{2B})_{z}-L³-L⁴-(L⁵)ₘ-(L⁶)ₙ-(L⁷)ₚ-R²;
L² is selected from a C₁₋₆ alkylene;
L^{2B} is selected from NR⁴C(O)O—C₁₋₆ alkylene-phenylene, wherein the phenylene is optionally substituted with one or more R⁵;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocyclene;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
R⁴ is selected from hydrogen and C₁₋₆ alkyl optionally substituted with one or more SO₂C₁₋₆ alkyl;
each R⁵ is independently selected from a sugar;
m is selected from 0 and 1;
n is selected from 0 and 1;
p is selected from 0 and 1;
q is selected from 0 to 8;
s is selected from 0 and 1; and
z is selected from 0 and 1.

In some embodiments, Formula (I) is represented by

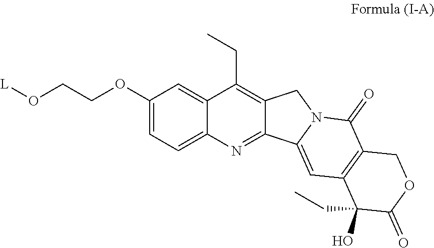

or a pharmaceutically acceptable salt thereof.

In some embodiments, Formula (I) is represented by

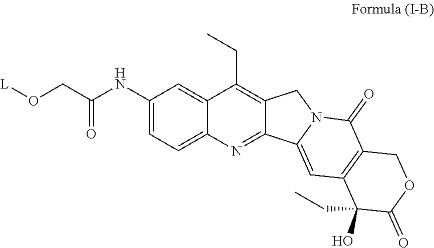

In some embodiments, for a compound or salt of Formula (I), Formula (I-A), Formula (I-B), Formula (II), or Formula (III), the L is L²-L³-L⁴-L⁵-L⁶-L⁷-R². In some cases, the L is L²-L³-L⁴-L⁵-L⁶-R². In some cases, the L is L²-L³-L⁴-L⁵-L⁶-R². In some cases, the L is L²-L³-L⁴-L⁷-R². In some cases, the linker is L²-L³-L⁴-R². In some cases, L is L²-L^{2B}-L³-L⁴-L⁵-R².

In some embodiments, for a compound or salt of Formula (I), Formula (I-A), Formula (I-B), or Formula (III), L² is selected from C₁₋₆ alkylene. In some cases, L² is C₅ alkylene. In some cases, L² is C₄ alkylene. In some cases, L² is C₃ alkylene. In some cases, L² is C₂ alkylene. In some cases, L² is C₁ alkylene.

In some embodiments, for a compound or salt of Formula (I), Formula (I-A), Formula (I-B), Formula (II), Formula (III), or Formula (III-A), z is 1. In some cases, L^{2B} is selected from NR⁴C(O)O—C₁₋₆ alkylene-phenyl, wherein the phenyl is optionally substituted with one R⁵. In some cases, L^{2B} is selected from NR⁴C(O)O—C₁₋₂ alkylene-phenyl, wherein the phenyl is optionally substituted with one R⁵. In some cases, L^{2B} is selected from NR⁴C(O)O—C₁ alkylene-phenyl, wherein the phenyl is optionally substituted with one R⁵. In some cases, L^{2B} is

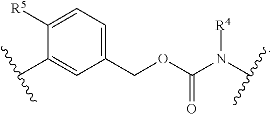

In some cases, L^{2B} is

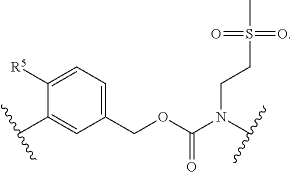

In some cases, L^{2B} is

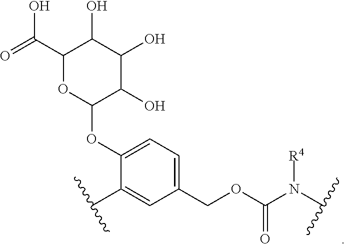

In some cases, L^{2B} is

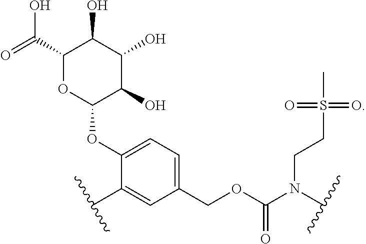 [SMILES: *c1cc(COC(=O)N(*)CCS(C)(=O)=O)ccc1O[C@@H]1O[C@H](C(=O)O)[C@@H](O)[C@H](O)[C@H]1O]

In some cases, each R⁵ is independently selected from a sugar. In some cases, the sugar is selected from a monosaccharide and disaccharides. In some cases, the sugar is a selected from a monosaccharide. In some cases, the sugar is selected from fructose, galactose, glucose, glucuronic acid, maltose, sucrose, and sucrose. In some cases, the sugar is glucuronic acid. In some cases, R⁵ is

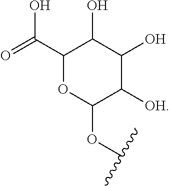 [SMILES: *OC1OC(C(=O)O)C(O)C(O)C1O]

In some cases, R⁵ is

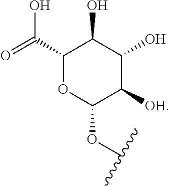 [SMILES: *O[C@@H]1O[C@H](C(=O)O)[C@@H](O)[C@H](O)[C@H]1O]

In some cases, z is 0. In some cases, R⁴ is selected from hydrogen. In some cases, R⁴ is selected from C₁₋₆ alkyl optionally substituted with one SO₂C₁₋₆ alkyl. In some cases, R⁴ is selected from C₁₋₆ alkyl substituted with one SO₂C₁₋₆ alkyl. In some cases, R⁴ is selected from C₁₋₆ alkyl substituted with one SO₂methyl. In some cases, R⁴ is

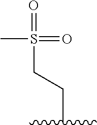 [SMILES: *CCS(C)(=O)=O]

In some embodiments, for a compound or salt of Formula (I), Formula (I-A), Formula (I-B), Formula (II), Formula (III), or Formula (III-A), L³ is selected from a residue comprising 1 to 7 amino acids. In some cases, L³ is selected from a residue comprising 1 to 5 amino acids. In some cases, L³ is selected from a residue comprising 1 to 4 amino acids. In some cases, L³ is selected from a residue comprising 1 to 3 amino acids. In some cases, L³ is selected from a residue comprising 1 to 2 amino acids. In some cases, L³ is selected from a residue comprising 2 to 4 amino acids. In some cases, L³ is selected from a residue comprising 1 amino acid. In some cases, L³ is selected from a residue comprising 2 amino acids. In some cases, L³ is selected from a residue comprising 3 amino acids. In some cases, L³ is selected from a residue comprising 4 amino acids. In some cases, L³ is selected from a residue comprising 5 amino acids. In some cases, L³ is selected from a residue comprising 6 amino acids. In some cases, L³ is selected from a residue comprising 7 amino acids.

In some embodiments, for a compound or salt of Formula (I), Formula (I-A), Formula (I-B), Formula (II), Formula (III), or Formula (III-A), L³ is selected from a residue comprising natural amino acids and unnatural amino acids.

In some embodiments, for a compound or salt of Formula (I), Formula (I-A), Formula (I-B), Formula (II), Formula (III), or Formula (III-A), L³ is selected from a residue comprising natural amino acids. In some cases, L³ is selected from a residue comprising alpha amino acids. In some cases, L³ is selected from a residue comprising beta amino acids.

In some embodiments, for a compound or salt of Formula (I), Formula (I-A), Formula (I-B), Formula (II), Formula (III), or Formula (III-A), the amino acid is an unnatural amino acid.

In some embodiments, for a compound or salt of Formula (I), Formula (I-A), Formula (I-B), Formula (II), Formula (III), or Formula (III-A), L³ is selected from a group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, citrulline, and β-Alanine. In some cases, L³ is selected from a group consisting of alanine, aspartic acid, cysteine, glutamic acid, glutamine, glycine, isoleucine, leucine, lysine, phenylalanine, serine, valine, citrulline, sarcosine, and β-Alanine. In some cases, L³ is selected from a group consisting of alanine, glycine, lysine, phenylalanine, serine, valine, citrulline, sarcosine, and β-Alanine. In some cases, L³ is selected from a group consisting of alanine, glycine, phenylalanine, valine, citrulline, and β-Alanine. In some cases, L³ is selected from a group consisting of alanine, valine, and citrulline. In some cases, L³ is selected from a group consisting of glycine and phenylalanine. In some cases, L³ is selected from a group consisting of glycine, phenylalanine, and sarcosine. In some cases, L³ is selected from a group consisting of sarcosine.

In some embodiments, for a compound or salt of Formula (I), Formula (I-A), Formula (I-B), Formula (II), Formula (III), or Formula (III-A), L³ comprises 4 amino acids. In some cases, L³ comprises 2 amino acids. In some cases, L³ comprises 1 amino acid. In some cases, L³ comprises at least two different amino acids. In some cases, L³ is

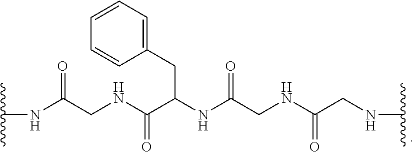 [SMILES: *NCC(=O)NCC(=O)NC(Cc1ccccc1)C(=O)NCC(=O)N*]

In some cases, L³ is

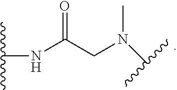 [SMILES: *NC(=O)CN(*)C]

In some embodiments, for a compound or salt of Formula (I), Formula (I-A), Formula (I-B), Formula (II), Formula (III), or Formula (III-A), L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl. In some cases, L⁴ is selected from an optionally substituted C₁₋₆ alkylene, wherein the C₁₋₆ alkylene is optionally substituted with one or more substituents independently selected from —OH, —NH₂, and oxo. In some cases, L⁴ is selected from an optionally substituted C₁₋₆ alkylene, wherein the C₁₋₆ alkylene is optionally substituted with oxo. In some cases, L⁴ is selected from —C(O)—(CH₂)₂— and —C(O)—(CH₂)₅—. In some cases, L⁴ is —C(O)—(CH₂)₂—. In some cases, L⁴ is —C(O)—(CH₂)₃—. In some cases, L⁴ is —C(O)—(CH₂)₄—. In some cases, L⁴ is —C(O)—(CH₂)₅—. In some cases, L⁴ is selected from

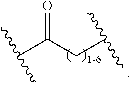

In some embodiments, for a compound or salt of Formula (I), Formula (I-A), Formula (I-B), Formula (II), Formula (III), or Formula (III-A), L⁵ is selected from an —(O—CH₂—CH₂—)_{q}—(NR³)ₛ—; wherein, q is selected from 0 to 8; and s is selected from 0 and 1. In some cases, L⁵ is selected from an —(O—CH₂—CH₂—)_{q}—(NR³)ₛ—; wherein, q is selected from 0 to 8; and s is 0. In some cases, L⁵ is selected from an —(O—CH₂—CH₂—)_{q}—(NR³)ₛ—; wherein, q is selected from 0 to 8; and s is 1. In some cases, q is 1. In some cases, q is 2. In some cases, q is 3. In some cases, q is 4. In some cases, q is 5. In some cases, q is 6. In some cases, L⁵ is —(O—CH₂—CH₂—)₂—NH—. In some cases, L⁵ is —(O—CH₂—CH₂—)₃—NH—. In some cases, L⁵ is —(O—CH₂—CH₂—)₄—NH—. In some cases, L⁵ is —(O—CH₂—CH₂—)₅—NH—. In some cases, L⁵ is —(O—CH₂—CH₂—)₆—NH—. In some cases, L⁵ is —(O—CH₂—CH₂—)₇—NH—. In some cases, L⁵ is —(O—CH₂—CH₂—)₈—NH—. In some cases, L⁵ is selected from an —(O—CH₂—CH₂—)₁-8. In some cases, s is 0. In some cases, s is 1.

In some embodiments, for a compound or salt of Formula (I), Formula (I-A), Formula (I-B), Formula (II), Formula (III), or Formula (III-A), L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl. In some cases, L⁶ is selected from an optionally substituted C₁₋₆ alkylene, wherein the C₁₋₆ alkylene is optionally substituted with one or more substituents independently selected from —OH, —NH₂, and oxo. In some cases, L⁶ is selected from an optionally substituted C₁₋₆ alkylene, wherein the C₁₋₆ alkylene is optionally substituted with oxo. In some cases, L⁶ is selected from —C(O)—(CH₂)₂— and —C(O)—(CH₂)₅—. In some cases, L⁶ is —C(O)—(CH₂)₂—. In some cases, L⁶ is —C(O)—(CH₂)₃—. In some cases, L⁶ is —C(O)—(CH₂)₄—. In some cases, L⁶ is —C(O)—(CH₂)₅—. In some cases, L⁶ is selected from

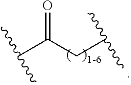

In some embodiments, for a compound or salt of Formula (I), Formula (I-A), Formula (I-B), Formula (II), Formula (III), or Formula (III-A), L⁷ is selected from a C₅₋₆ carbocycle. In some cases, L⁷ is selected from a C₅₋₆ carbocyclene. In some cases, L⁷ is selected from a C₆ carbocycle. In some cases, L⁷ is selected from a C₆ carbocyclene. In some cases, L⁷ is phenylene. In some cases, L⁷ is cyclohexylene. In some cases, L⁷ is

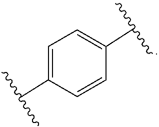 [SMILES: *c1ccc(*)cc1]

In some cases, L⁷ is

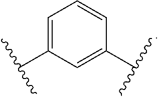 [SMILES: *c1cccc(*)c1]

In some embodiments, for a compound or salt of Formula (I), Formula (I-A), Formula (I-B), Formula (II), or Formula (III), R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂-10 alkenyl, C₂-10 alkynyl. In some cases, R² is selected from an optionally substituted 5- to 6-membered heterocyclene, wherein the 5- to 6-membered heterocyclene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl. In some cases, R² is selected from an optionally substituted 5- to 6-membered heterocycle, wherein the 5- to 6-membered heterocycle is optionally substituted with one or more substituents independently selected from oxo and halogen. In some cases, R² is selected from an optionally substituted 5- to 6-membered heterocyclene, wherein the 5- to 6-membered heterocyclene is optionally substituted with one or more substituents independently selected from oxo and halogen. In some cases, R² is selected from an optionally substituted 5-membered heterocycle, wherein the 5-membered heterocycle is optionally substituted with one or more substituents independently selected from oxo. In some cases, R² is selected from an optionally substituted 5-membered heterocyclene, wherein the 5-membered heterocyclene is optionally substituted with one or more substituents independently selected from oxo. In some cases, the 5-membered heterocycle has at least one double bond. In some cases, the 5-membered heterocycle has one double bond. In some cases, R² is a maleimide. In some cases, R² is

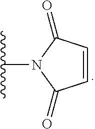 [SMILES: *N1C(=O)C=CC1=O]

In some cases, R² is

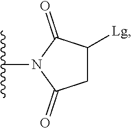

wherein Lg is a ligand. In some cases, R² is a reactive moiety capable of forming a new bond. In some cases, R² is a reactive moiety capable of forming a new bond from an existing double bond.

In some embodiments, for a compound or salt of Formula (I), Formula (I-A), Formula (I-B), Formula (II), Formula (III), or Formula (III-A), L²-L³-L⁴-R² is selected from

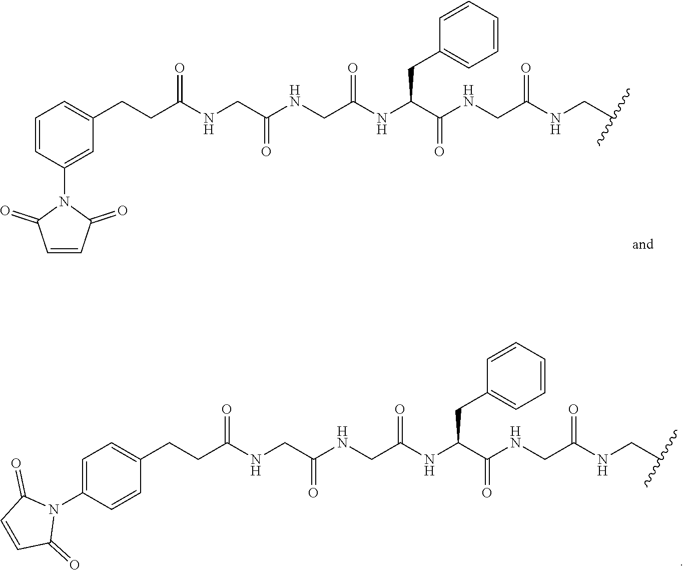 [SMILES: *CNC(=O)CNC(=O)[C@H](Cc1ccccc1)NC(=O)CNC(=O)CNC(=O)CCc1cccc(N2C(=O)C=CC2=O)c1 ; *CNC(=O)CNC(=O)[C@H](Cc1ccccc1)NC(=O)CNC(=O)CNC(=O)CCc1ccc(N2C(=O)C=CC2=O)cc1]

In some cases, L²-L³-L⁴ is

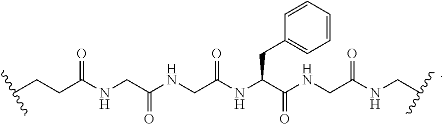 [SMILES: *CCC(=O)NCC(=O)NCC(=O)N[C@@H](Cc1ccccc1)C(=O)NCC(=O)NC*]

In some embodiments, for a compound or salt or Formula (I), Formula (I-A), Formula (I-B), Formula (II), Formula (III), or Formula (III-A), L²-L³-L⁴ is

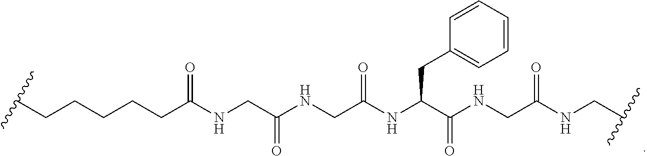 [SMILES: *CCCCCC(=O)NCC(=O)NCC(=O)N[C@@H](Cc1ccccc1)C(=O)NCC(=O)NC*]

In some cases, L²-L³ is

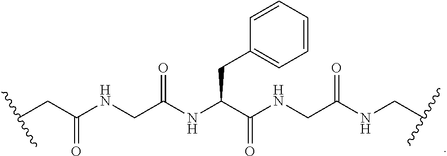 [SMILES: *CNC(=O)CNC(=O)[C@H](Cc1ccccc1)NC(=O)CNC(=O)C*]

In some embodiments, for a compound or salt of Formula (I), Formula (I-A), Formula (I-B), Formula (II), or Formula (III), L²-L³-L⁴-L⁵-L⁶-L⁷-R² is

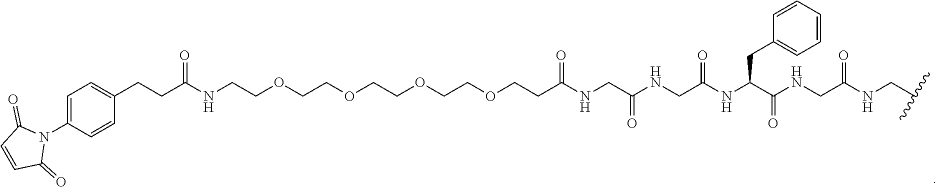 [SMILES: *CNC(=O)CNC(=O)[C@H](Cc1ccccc1)NC(=O)CNC(=O)CNC(=O)CCOCCOCCOCCOCCNC(=O)CCc1ccc(N2C(=O)C=CC2=O)cc1]

In some cases, L²-L³-L⁴-L⁵-L⁶-L⁷ is

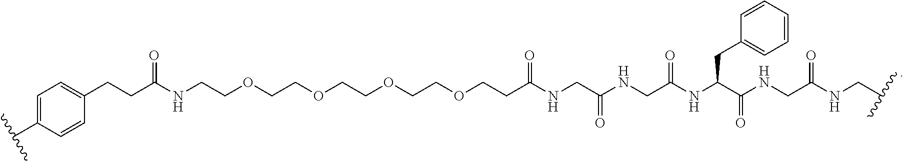 [SMILES: *CNC(=O)CNC(=O)[C@H](Cc1ccccc1)NC(=O)CNC(=O)CNC(=O)CCOCCOCCOCCOCCNC(=O)CCc1ccc(*)cc1]

In some cases, L²-L³-L⁴-L⁵-L⁶ is

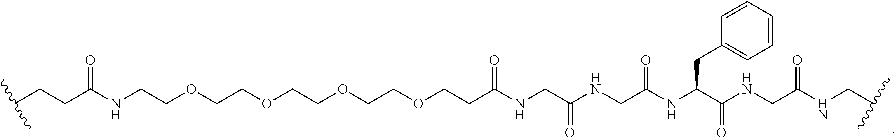 [SMILES: *CCC(=O)NCCOCCOCCOCCOCCC(=O)NCC(=O)NCC(=O)N[C@@H](Cc1ccccc1)C(=O)NCC(=O)NC*]

In some cases, L²-L³-L⁴-L⁵ is

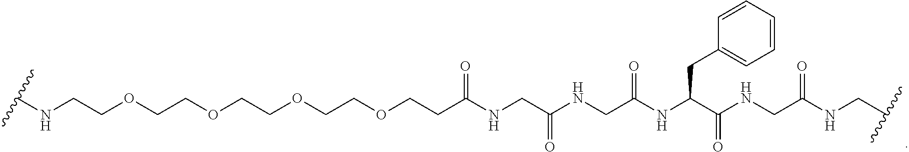 [SMILES: *CNC(=O)CNC(=O)[C@H](Cc1ccccc1)NC(=O)CNC(=O)CNC(=O)CCOCCOCCOCCOCCN*]

In some cases, L²-L³-L⁴-L⁵ is

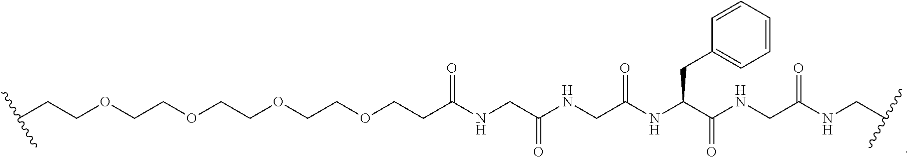 [SMILES: *CCOCCOCCOCCOCCC(=O)NCC(=O)NCC(=O)N[C@@H](Cc1ccccc1)C(=O)NCC(=O)NC*]

In some embodiments, for a compound or salt of Formula (I), Formula (I-A), Formula (I-B), Formula (II), or Formula (III), L²-L^{2B}-L³-L⁴-R² is

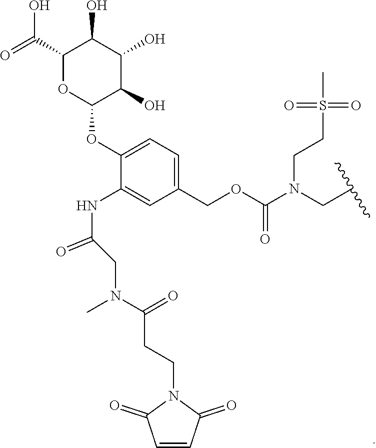 [SMILES: *CN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1]

In some cases, L²-L^{2B}-L³-L⁴ is

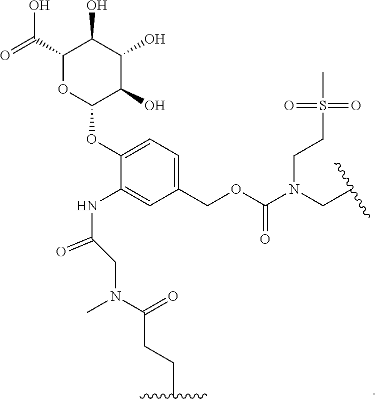 [SMILES: *CCC(=O)N(C)CC(=O)Nc1cc(COC(=O)N(C*)CCS(C)(=O)=O)ccc1O[C@@H]1O[C@H](C(=O)O)[C@@H](O)[C@H](O)[C@H]1O]

In some cases, L²-L^{2B}-L³ is

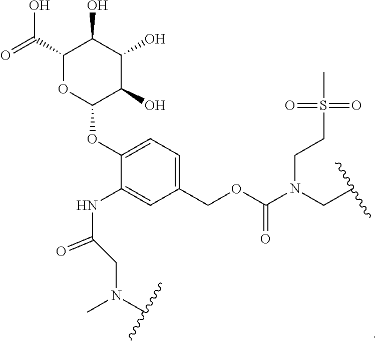 [SMILES: *CN(CCS(C)(=O)=O)C(=O)OCc1ccc(O[C@@H]2O[C@H](C(=O)O)[C@@H](O)[C@H](O)[C@H]2O)c(NC(=O)CN(*)C)c1]

In some cases, L²-L^{2B} is

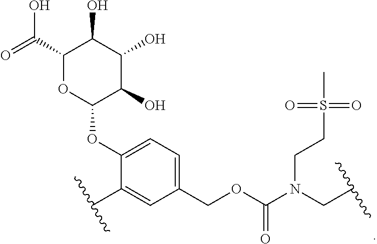 [SMILES: *CN(CCS(C)(=O)=O)C(=O)OCc1ccc(O[C@@H]2O[C@H](C(=O)O)[C@@H](O)[C@H](O)[C@H]2O)c(*)c1]

In some cases, L^{2B} is

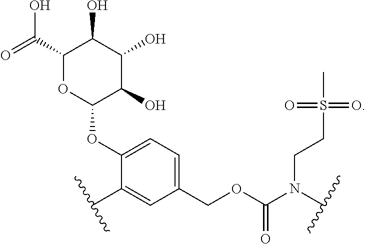 [SMILES: *c1cc(COC(=O)N(*)CCS(C)(=O)=O)ccc1O[C@@H]1O[C@H](C(=O)O)[C@@H](O)[C@H](O)[C@H]1O]

In some embodiments, for a compound or salt of Formula (I), Formula (I-A), Formula (I-B), Formula (II), or Formula (III), L is

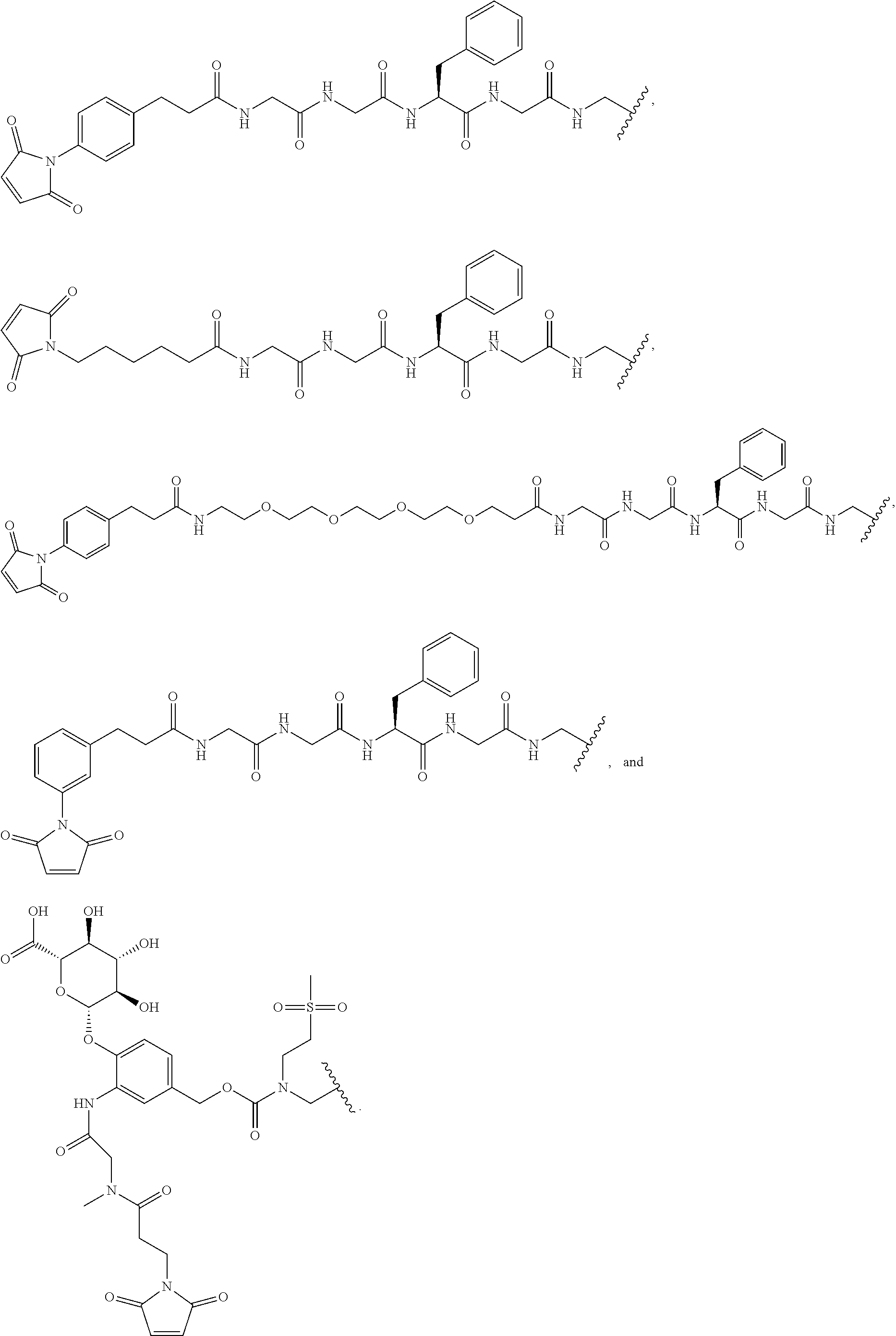

In some embodiments, Formula (I) is represented by

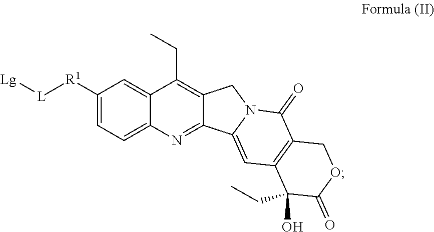

or a pharmaceutically acceptable salt thereof, wherein Lg is the ligand.

In an aspect, the present disclosure provides a compound of Formula (III):

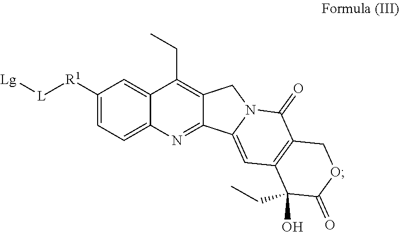

or a pharmaceutically acceptable salt thereof, wherein;
Lg is a ligand
R¹ is selected from —O—CH₂—CH₂—O— and —NH—C(O)—CH₂—O—;
L is L²-(L²B)_{z}-L³-L⁴-(L⁵)ₘ-(L⁶)ₙ-(L⁷)ₚ-R²;
L² is selected from a C₁₋₆ alkylene;
L³ is selected from a residue comprising 1 to 7 amino acids;
   L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an —(O—CH₂—CH₂—)_{q}—(NR³)ₛ—;
   L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   L⁷ is selected from a C₅₋₆ carbocycle;
      R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
      R³ is selected from hydrogen and C₁₋₆ alkyl;
      R⁴ is selected from C₁₋₆ alkyl optionally substituted with one or more SO₂C₁₋₆ alkyl;
      each R⁵ is independently selected from a sugar;
m is selected from 0 and 1;
n is selected from 0 and 1;
p is selected from 0 and 1;
q is selected from 0 to 8;
s is selected from 0 and 1; and
z is selected from 0 and 1.

In some embodiments, Formula (III) is represented by

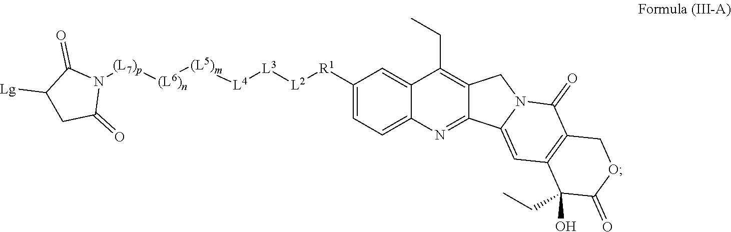

or a pharmaceutically acceptable salt thereof.

In an aspect, the present disclosure provides a compound of Formula (IV)

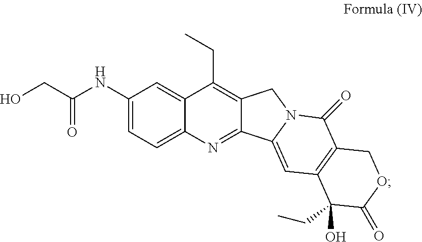 [SMILES: CCc1c2c(nc3ccc(NC(=O)CO)cc13)-c1cc3c(c(=O)n1C2)COC(=O)[C@]3(O)CC]

or a pharmaceutically acceptable salt thereof.

In some embodiments, for a compound or salt of Formula (I), Formula (I-A), or Formula (I-B), R¹ is selected from —O—CH₂—CH₂—O—;
L is L²-L³-L⁴-(L⁵)ₘ-(L⁶)ₙ-(L⁷)ₚ-R²;
   L² is selected from a C₁₋₆ alkylene;
   L³ is selected from a residue comprising 3 to 5 amino acids;
   L⁴ is selected from

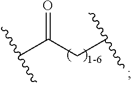

L⁵ is selected from an (O—CH₂—CH₂—)_{q}—(NR³)ₛ;
L⁶ is selected from

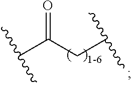

L⁷ is phenylene;
R² is selected from maleimide;
R³ is selected from hydrogen and C₁₋₆ alkyl;
m is selected from 0 and 1;
n is selected from 0 and 1;
p is selected from 0 and 1;
q is selected from 2 to 6; and
s is selected from 0 and 1.

In some embodiments, for a compound or salt of Formula (I), Formula (I-A), or Formula (I-B), R¹ is selected from —O—CH₂—CH₂—O—;
L is L²-L³-L⁴-(L⁵)ₘ-(L⁶)ₙ-(L⁷)ₚ-R²;
   L² is

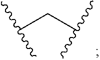 [SMILES: *C*]

L³ is

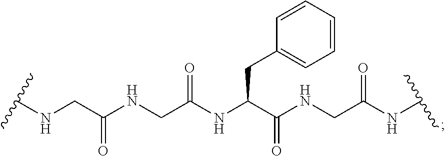 [SMILES: *NCC(=O)NCC(=O)N[C@@H](Cc1ccccc1)C(=O)NCC(=O)N*]

L⁴ is selected from

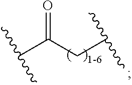

L⁵ is

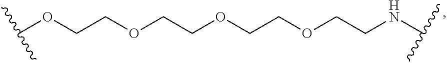 [SMILES: *NCCOCCOCCOCCO*]

m is selected from 0 and 1;
L⁶

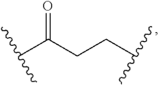 [SMILES: *CCC(*)=O]

n is selected form 0 and 1;
L⁷ is selected from

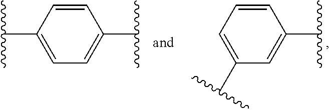 [SMILES: *c1ccc(*)cc1 ; *c1cccc(*)c1]

p is selected from 0 and 1;
R² is

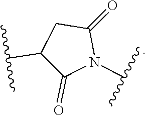 [SMILES: *C1CC(=O)N(*)C1=O]

In some embodiments, for a compound or salt of Formula (I), Formula (I-A), or Formula (I-B), R¹ is selected from —O—CH₂—CH₂—O—;
L is L²-L³-L⁴-(L⁵)ₘ-(L⁶)ₙ-(L⁷)ₚ-R²;
   L² is

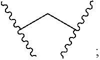 [SMILES: *C*]

L³ is

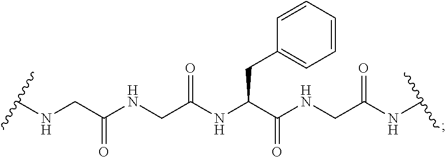 [SMILES: *NCC(=O)NCC(=O)N[C@@H](Cc1ccccc1)C(=O)NCC(=O)N*]

L⁴ is selected from

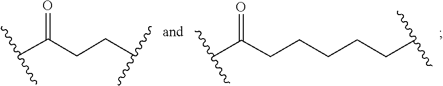 [SMILES: *CCC(*)=O ; *CCCCCC(*)=O]

L⁵ is

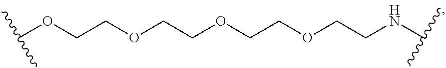 [SMILES: *NCCOCCOCCOCCO*]

m is selected from 0 and 1;
L⁶

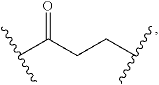 [SMILES: *CCC(*)=O]

n is selected form 0 and 1;
L⁷

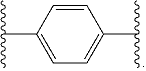 [SMILES: *c1ccc(*)cc1]

p is selected from 0 and 1;
R²

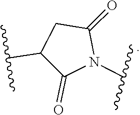 [SMILES: *C1CC(=O)N(*)C1=O]

In some embodiments, Formula (III) is represented by Formula (V-A)

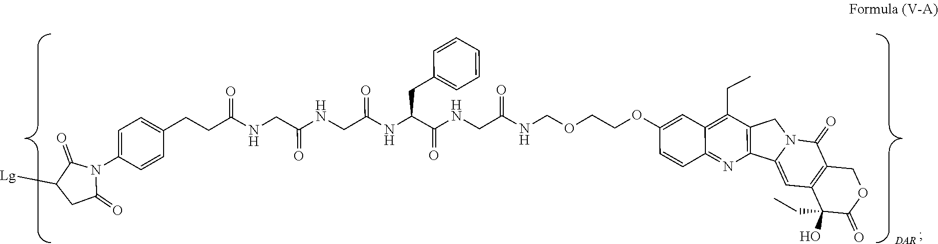

or a pharmaceutically acceptable salt thereof, wherein the DAR is selected from about 1-8. In some cases, the DAR is about 8.

In some embodiments, Formula (III) is represented by Formula (V-B)

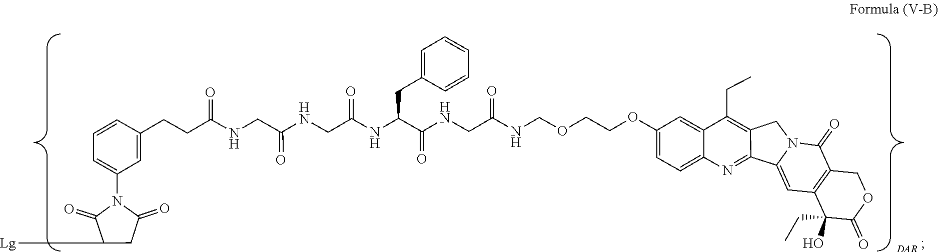

or a pharmaceutically acceptable salt thereof, wherein the DAR is selected from about 1-8. In some cases, the DAR is about 8.

In some embodiments, Formula (III) is represented by Formula (V-C):

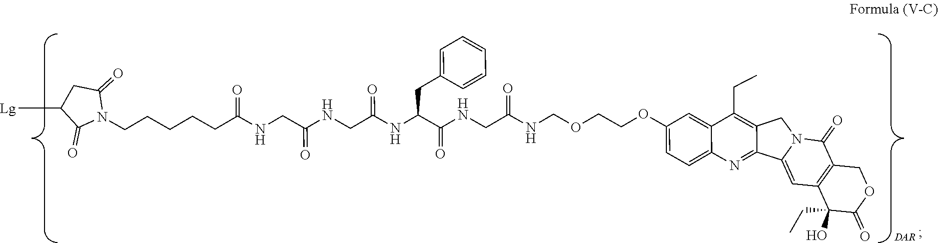

or a pharmaceutically acceptable salt thereof, wherein the DAR is selected from about 1-8. In some cases, the DAR is about 8.

In some embodiments, Formula (III) is represented by Formula (V-D)

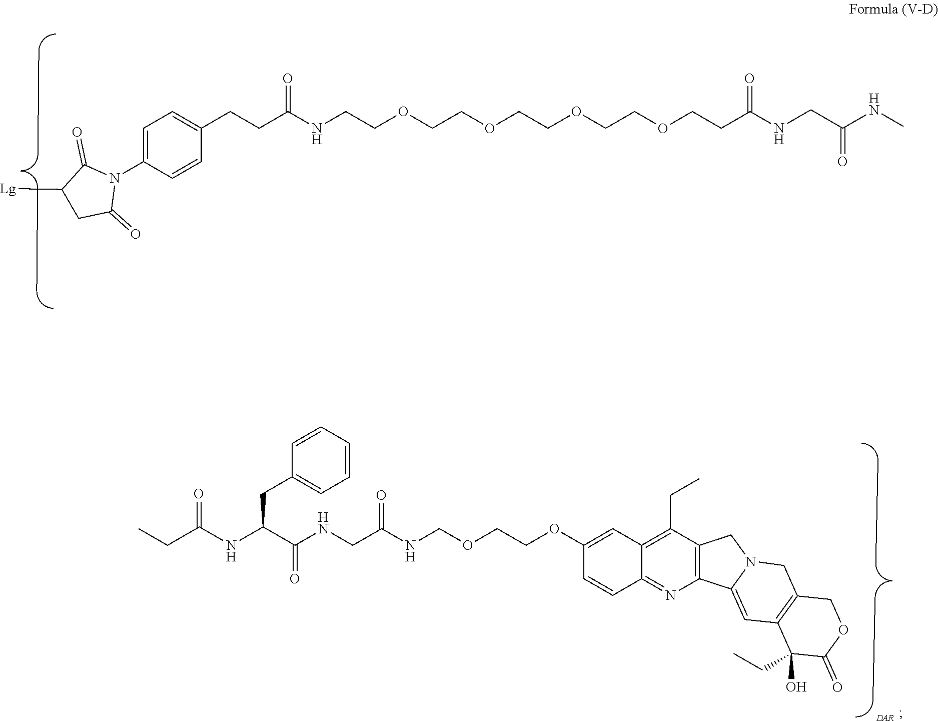

or a pharmaceutically acceptable salt thereof, wherein the DAR is selected from about 1-8. In some cases, the DAR is about 8.

In some embodiments, the compound of Formula (III) is selected from:

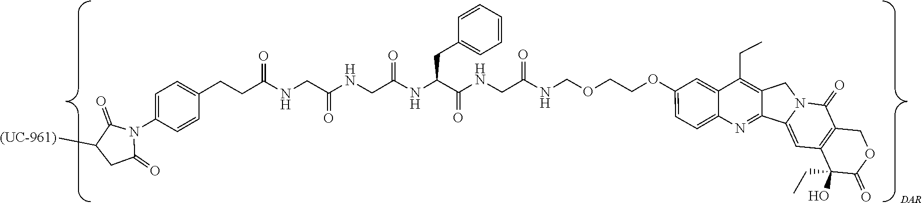

or a pharmaceutically acceptable salt thereof, wherein the DAR is selected from about 1-8. In some cases, the DAR is about 8.

In some embodiments, the compound of Formula (III) is selected from:

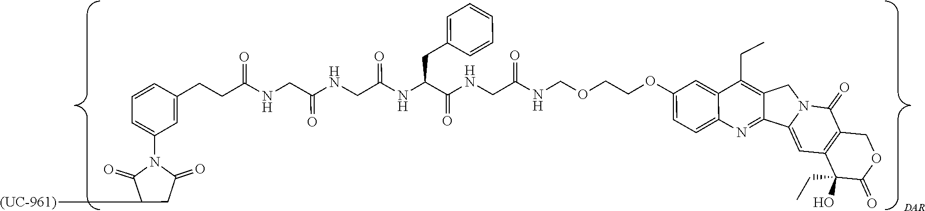

or a pharmaceutically acceptable salt thereof, wherein the DAR is selected from about 1-8. In some cases, the DAR is about 8.

In some embodiments, the compound of Formula (III) is selected from:

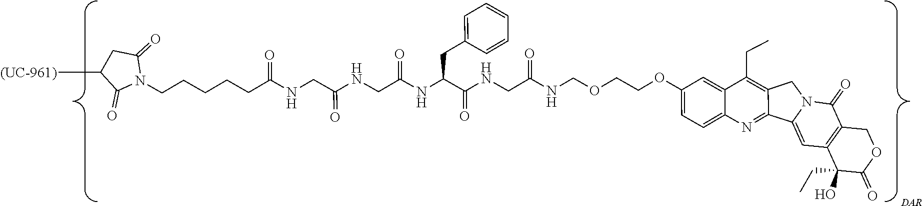

or a pharmaceutically acceptable salt thereof, wherein the DAR is selected from about 1-8.

In some embodiments, Formula (III) or Formula (IV)* includes a DAR value. In some cases, the DAR is a drug to antibody ratio. In some cases, the DAR is the average number of drugs conjugated to the ligand (Lg). In some cases, the DAR is determined using RP-HPLC. In some cases, the DAR is determined using RP-HPLC as in example 17. In some cases, the DAR is at most about 16. In some cases, the DAR is about 8. In some cases, the DAR is most about 10. In some cases, the DAR is at least about 1. In some cases, the DAR is at least about 2. In some cases, the DAR is at least about 4. In some cases, the DAR is at least about 8. In some cases, the DAR is at most about 8. In some cases, the DAR is from about 1 to about 16. In some cases, the DAR is from about 4 to about 12. In some cases, the DAR is from about 6 to about 10. In some cases, the DAR is from about 7 to about 9. In some cases, the DAR is from about 8 to about 10. In some cases, the DAR is from about 6 to about 8. In some cases, the DAR is about 8.

In some embodiments, for a compound or salt of Formula (III), Formula (V-A), Formula (V-B), Formula (V-C), or Formula (V-D), DAR is a drug to antibody ratio. In some cases, DAR is the average number of drugs conjugated to the ligand (Lg). In some cases, the DAR is determined using RP-HPLC. In some cases, the DAR is determined using RP-HPLC as in example 17. In some cases, the DAR is at most about 16. In some cases, the DAR is about 8. In some cases, the DAR is most about 10. In some cases, the DAR is at least about 1. In some cases, the DAR is at least about 2. In some cases, the DAR is at least about 4. In some cases, the DAR is at least about 8. In some cases, the DAR is at most about 8. In some cases, the DAR is from about 1 to about 16. In some cases, the DAR is from about 4 to about 12. In some cases, the DAR is from about 6 to about 10. In some cases, the DAR is from about 7 to about 9. In some cases, the DAR is from about 8 to about 10. In some cases, the DAR is from about 6 to about 8. In some cases, the DAR is about 8.

In some embodiments, for a compound or salt of Formula (I), Formula (I-A), or Formula (I-B), Formula (III), Formula (V-A), Formula (V-B), Formula (V-C), Formula (V-D), or Formula (IV)*, UC-961, PTK-7, trastuzumab, brentuximab, loncastuximab, rosopatamab, rituximab, pinatuzumab, polatuzumab, and naratuximab. In some cases, the ligand is selected from trastuzumab, brentuximab, loncastuximab, rosopatamab, rituximab, pinatuzumab, polatuzumab, and naratuximab. In some cases, the ligand is trastuzumab.

In an aspect, the present disclosure provides a compound represented by Formula (I)*,

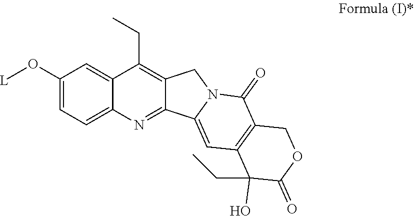

or a pharmaceutically acceptable salt thereof, wherein;
   L is L²-L³-L⁴-L)ₘ L⁶)ₙ-(L⁷)ₚ-R²;
   L² is selected from a C₁₋₆ alkylene;
   L³ is selected from a residue comprising 1 to 7 amino acids;
   L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
   L⁵ is selected from an (O—CH₂—CH₂—)_{q}—(NR³)ₛ;
   L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   L⁷ is selected from a C₅₋₆ carbocyclene;
   R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
   R³ is selected from hydrogen and C₁₋₆ alkyl;
   m is selected from 0 and 1;
n is selected from 0 and 1;
p is selected from 0 and 1;
q is selected from 0 to 8; and
s is selected from 0 and 1.

In some embodiments, Formula (I)* is represented by

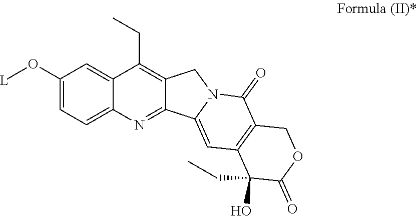

or a pharmaceutically acceptable salt thereof.

In some embodiments, Formula (I)* is represented by

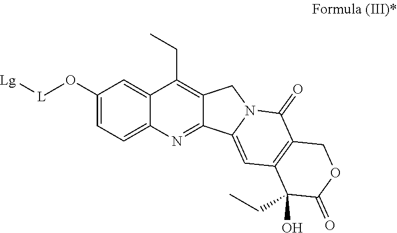

or a pharmaceutically acceptable salt thereof, wherein;
Lg is the ligand.

In some embodiments, for a compound or salt of Formula (I)*, Formula (II)*, Formula (III)*, or Formula (IV)*, the L is L²-L³-L⁴-L⁵-L⁶-L⁷-R². In some cases, the L is L²-L³-L⁴-L⁵-L⁶-R². In some cases, the L is L²-L³-L⁴-L⁶-R². In some cases, the L is L²-L³-L⁶-L⁷-R². In some cases, the linker is L²-L³-L⁴-R². In some cases, L is L²-L⁴-R².

In some embodiments, for a compound or salt of Formula (I)*, Formula (II)*, Formula (III)*, Formula (IV)*, or Formula (IV-A)*, L² is selected from C₁₋₆ alkylene. In some cases, L² is C₅ alkylene. In some cases, L² is C₄ alkylene. In some cases, L² is C₃ alkylene. In some cases, L² is C₂ alkylene. In some cases, L² is C₁ alkylene.

In some embodiments, for a compound or salt of Formula (I)*, Formula (II)*, Formula (III)*, Formula (IV)*, or Formula (IV-A)*, L³ is selected from a residue comprising 1 to 7 amino acids. In some cases, L³ is selected from a residue comprising 1 to 5 amino acids. In some cases, L³ is selected from a residue comprising 1 to 4 amino acids. In some cases, L³ is selected from a residue comprising 1 to 3 amino acids. In some cases, L³ is selected from a residue comprising 1 to 2 amino acids. In some cases, L³ is selected from a residue comprising 2 to 4 amino acids. In some cases, L³ is selected from a residue comprising 1 amino acid. In some cases, L³ is selected from a residue comprising 2 amino acids. In some cases, L³ is selected from a residue comprising 3 amino acids. In some cases, L³ is selected from a residue comprising 4 amino acids. In some cases, L³ is selected from a residue comprising 5 amino acids. In some cases, L³ is selected from a residue comprising 6 amino acids. In some cases, L³ is selected from a residue comprising 7 amino acids.

In some embodiments, for a compound or salt of Formula (I)*, Formula (II)*, Formula (III)*, Formula (IV)*, or Formula (IV-A)*, L³ is selected from a residue comprising natural amino acids and unnatural amino acids.

In some embodiments, for a compound or salt of Formula (I)*, Formula (II)*, Formula (III)*, Formula (IV)*, or Formula (IV-A)*, L³ is selected from a residue comprising natural amino acids. In some cases, L³ is selected from a residue comprising alpha amino acids. In some cases, L³ is selected from a residue comprising beta amino acids.

In some embodiments, for a compound or salt of Formula (I)*, Formula (II)*, Formula (III)*, Formula (IV)*, or Formula (IV-A)*, the amino acid is an unnatural amino acid.

In some embodiments, for a compound or salt of Formula (I)*, Formula (II)*, Formula (III)*, Formula (IV)*, or Formula (IV-A)*, L³ is selected from a group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, citrulline, and β-Alanine. In some cases, L³ is selected from a group consisting of alanine, aspartic acid, cysteine, glutamic acid, glutamine, glycine, isoleucine, leucine, lysine, phenylalanine, serine, valine, citrulline, sarcosine, and β-Alanine. In some cases, L³ is selected from a group consisting of alanine, glycine, lysine, phenylalanine, serine, valine, citrulline, sarcosine, and β-Alanine. In some cases, L³ is selected from a group consisting of alanine, glycine, phenylalanine, valine, citrulline, and β-Alanine. In some cases, L³ is selected from a group consisting of alanine, valine, and citrulline. In some cases, L³ is selected from a group consisting of glycine and phenylalanine. In some cases, L³ is selected from a group consisting of glycine, phenylalanine, and sarcosine. In some cases, L³ is selected from a group consisting of sarcosine.

In some embodiments, for a compound or salt of Formula (I)*, Formula (II)*, Formula (III)*, Formula (IV)*, or Formula (IV-A)*, L³ comprises 4 amino acids. In some cases, L³ comprises 2 amino acids. In some cases, L³ comprises 1 amino acid. In some cases, L³ comprises at least two different amino acids. In some cases, L³ is

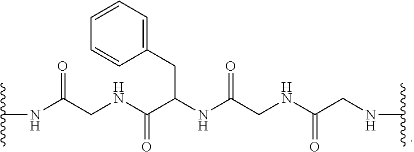 [SMILES: *NCC(=O)NCC(=O)NC(Cc1ccccc1)C(=O)NCC(=O)N*]

In some embodiments, for a compound or salt of Formula (I)*, Formula (II)*, Formula (III)*, Formula (IV)*, or Formula (IV-A)*, L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl. In some cases, L⁴ is selected from an optionally substituted C₁₋₆ alkylene, wherein the C₁₋₆ alkylene is optionally substituted with one or more substituents independently selected from —OH, —NH₂, and oxo. In some cases, L⁴ is selected from an optionally substituted C₁₋₆ alkylene, wherein the C₁₋₆ alkylene is optionally substituted with oxo. In some cases, L⁴ is selected from —C(O)—(CH₂)₂— and —C(O)—(CH₂)₅—. In some cases, L⁴ is —C(O)—(CH₂)₂—. In some cases, L⁴ is —C(O)—(CH₂)₃—. In some cases, L⁴ is —C(O)—(CH₂)₄—. In some cases, L⁴ is —C(O)—(CH₂)₅—. In some cases, L⁴ is

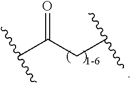

In some embodiments, for a compound or salt of Formula (I)*, Formula (II)*, Formula (III)*, Formula (IV)*, or Formula (IV-A)*, L⁵ is selected from an —(O—CH₂—CH₂—)_{q}—(NR³)ₛ—; wherein, q is selected from 0 to 8; and s is selected from 0 and 1. In some cases, L⁵ is selected from an —(O—CH₂—CH₂—)_{q}—(NR³)ₛ—; wherein, q is selected from 0 to 8; and s is 0. In some cases, L⁵ is selected from an —(O—CH₂—CH₂—)_{q}—(NR³)ₛ—; wherein, q is selected from 0 to 8; and s is 1. In some cases, q is 1. In some cases, q is 2. In some cases, q is 3. In some cases, q is 4. In some cases, q is 5. In some cases, q is 6. In some cases, L⁵ is —(O—CH₂—CH₂—)₂—NH—. In some cases, L⁵ is —(O—CH₂—CH₂—)₃—NH—. In some cases, L⁵ is —(O—CH₂—CH₂—)₄—NH—. In some cases, L⁵ is —(O—CH₂—CH₂—)₅—NH—. In some cases, L⁵ is —(O—CH₂—CH₂—)₆—NH—. In some cases, L⁵ is —(O—CH₂—CH₂—)₇—NH—. In some cases, L⁵ is —(O—CH₂—CH₂—)₈—NH—. In some cases, L⁵ is selected from an —(O—CH₂—CH₂—)₁₋₈. In some cases, s is 0. In some cases, s is 1.

In some embodiments, for a compound or salt of Formula (I)*, Formula (II)*, Formula (III)*, Formula (IV)*, or Formula (IV-A)*, L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl. In some cases, L⁶ is selected from an optionally substituted C₁₋₆ alkylene, wherein the C₁₋₆ alkylene is optionally substituted with one or more substituents independently selected from —OH, —NH₂, and oxo. In some cases, L⁶ is selected from an optionally substituted C₁₋₆ alkylene, wherein the C₁₋₆ alkylene is optionally substituted with oxo. In some cases, L⁶ is selected from —C(O)—(CH₂)₂— and —C(O)—(CH₂)₅—. In some cases, L⁶ is —C(O)—(CH₂)₂—. In some cases, L⁶ is —C(O)—(CH₂)₃—. In some cases, L⁶ is —C(O)—(CH₂)₄—. In some cases, L⁶ is —C(O)—(CH₂)₅—. In some cases, L⁶ is selected from

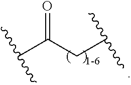

In some embodiments, for a compound or salt of Formula (I)*, Formula (II)*, Formula (III)*, Formula (IV)*, or Formula (IV-A)*, L⁷ is selected from a C₅₋₆ carbocycle. In some cases, L⁷ is selected from a C₆ carbocycle. In some cases, L⁷ is selected from a C₆ carbocyclene. In some cases, L⁷ is phenylene. In some cases, L⁷ is cyclohexylene. In some cases, L⁷ is

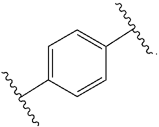 [SMILES: *c1ccc(*)cc1]

In some cases, L⁷ is

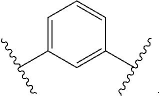 [SMILES: *c1cccc(*)c1]

In some cases, p is 0. In some cases, p is 1.

In some embodiments, for a compound or salt of Formula (I)*, Formula (II)*, Formula (III)*, or Formula (IV)*, R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl. In some cases, R² is selected from an optionally substituted 5- to 6-membered heterocyclene, wherein the 5- to 6-membered heterocyclene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl. In some cases, R² is selected from an optionally substituted 5- to 6-membered heterocycle, wherein the 5- to 6-membered heterocycle is optionally substituted with one or more substituents independently selected from oxo and halogen. In some cases, R² is selected from an optionally substituted 5- to 6-membered heterocyclene, wherein the 5- to 6-membered heterocyclene is optionally substituted with one or more substituents independently selected from oxo. In some cases, R² is selected from an optionally substituted 5-membered heterocycle, wherein the 5-membered heterocycle is optionally substituted with one or more substituents independently selected from oxo. In some cases, the 5-membered heterocycle has at least one double bond. In some cases, the 5-membered heterocycle has one double bond. In some cases, R² is a maleimide. In some cases, R² is

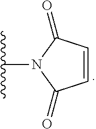 [SMILES: *N1C(=O)C=CC1=O]

In some cases, R² is

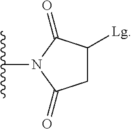

In some cases, R² is a reactive moiety capable of forming a new bond. In some cases, R² is a reactive moiety capable of forming a new bond from an existing double bond.

In some embodiments, for a compound or salt of Formula (I)*, Formula (II)*, Formula (III)*, or Formula (IV)*, L²-L³-L⁴-R² is selected from

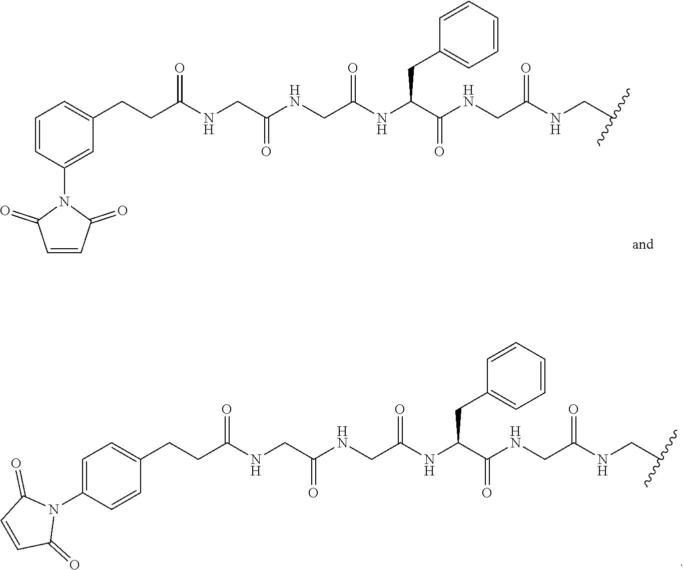 [SMILES: *CNC(=O)CNC(=O)[C@H](Cc1ccccc1)NC(=O)CNC(=O)CNC(=O)CCc1cccc(N2C(=O)C=CC2=O)c1 ; *CNC(=O)CNC(=O)[C@H](Cc1ccccc1)NC(=O)CNC(=O)CNC(=O)CCc1ccc(N2C(=O)C=CC2=O)cc1]

In some embodiments, for a compound or salt of Formula (I)*, Formula (II)*, Formula (III)*, Formula (IV)*, or Formula (IV-A)*, In some cases, L²-L³-L⁴ is

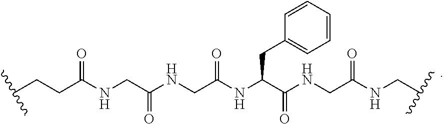 [SMILES: *CCC(=O)NCC(=O)NCC(=O)N[C@@H](Cc1ccccc1)C(=O)NCC(=O)NC*]

In some embodiments, for a compound or salt of Formula (I)*, Formula (II)*, Formula (III)*, Formula (IV)*, or Formula (IV-A)*, L²-L³-L⁴ is

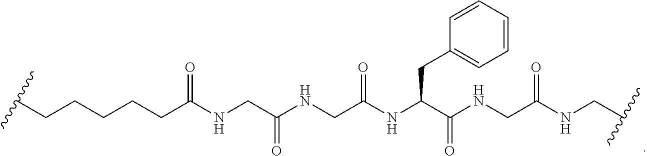 [SMILES: *CCCCCC(=O)NCC(=O)NCC(=O)N[C@@H](Cc1ccccc1)C(=O)NCC(=O)NC*]

In some cases, L²-L³ is

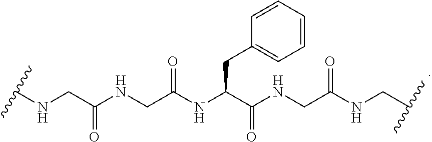 [SMILES: *CNC(=O)CNC(=O)[C@H](Cc1ccccc1)NC(=O)CNC(=O)CN*]

In some embodiments, for a compound or salt of Formula (I)*, Formula (II)*, Formula (III)*, or Formula (IV)*, L²-L³-L⁴-L⁵-L⁶-L⁷-R² is

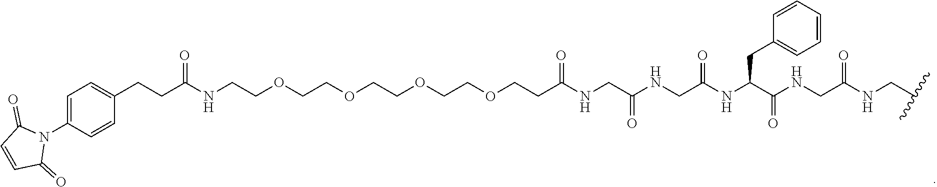 [SMILES: *CNC(=O)CNC(=O)[C@H](Cc1ccccc1)NC(=O)CNC(=O)CNC(=O)CCOCCOCCOCCOCCNC(=O)CCc1ccc(N2C(=O)C=CC2=O)cc1]

L³-L⁴-L⁵-L⁶-L⁷ is

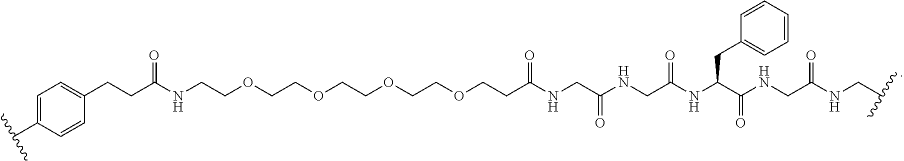 [SMILES: *CNC(=O)CNC(=O)[C@H](Cc1ccccc1)NC(=O)CNC(=O)CNC(=O)CCOCCOCCOCCOCCNC(=O)CCc1ccc(*)cc1]

In some embodiments, for a compound or salt of Formula (I)*, Formula (II)*, Formula (III)* Formula (IV)*, or Formula (IV-A)*, L²-L³-L⁴-L⁵-L⁶ is

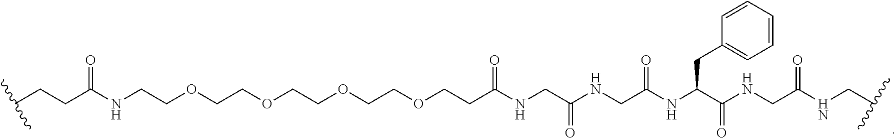 [SMILES: *CCC(=O)NCCOCCOCCOCCOCCC(=O)NCC(=O)NCC(=O)N[C@@H](Cc1ccccc1)C(=O)NCC(=O)NC*]

In some cases, L²-L³-L⁴-L⁵

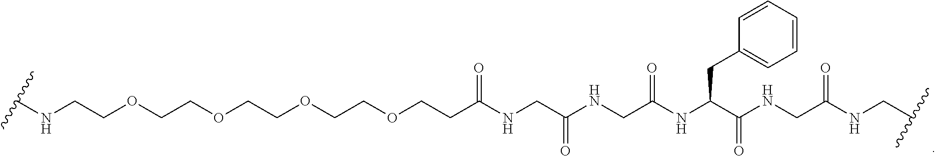 [SMILES: *CNC(=O)CNC(=O)[C@H](Cc1ccccc1)NC(=O)CNC(=O)CNC(=O)CCOCCOCCOCCOCCN*]

In some cases, L²-L³-L⁴-L⁵ is

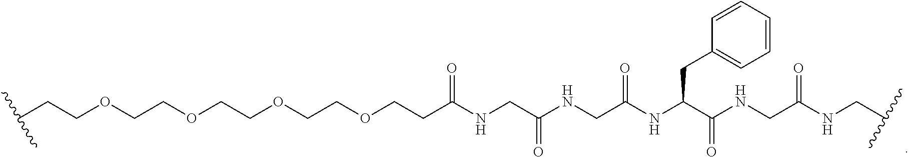 [SMILES: *CCOCCOCCOCCOCCC(=O)NCC(=O)NCC(=O)N[C@@H](Cc1ccccc1)C(=O)NCC(=O)NC*]

In some embodiments, for a compound or salt of Formula (I)*, Formula (II)*, Formula (III)*, or Formula (IV)*, L is selected from

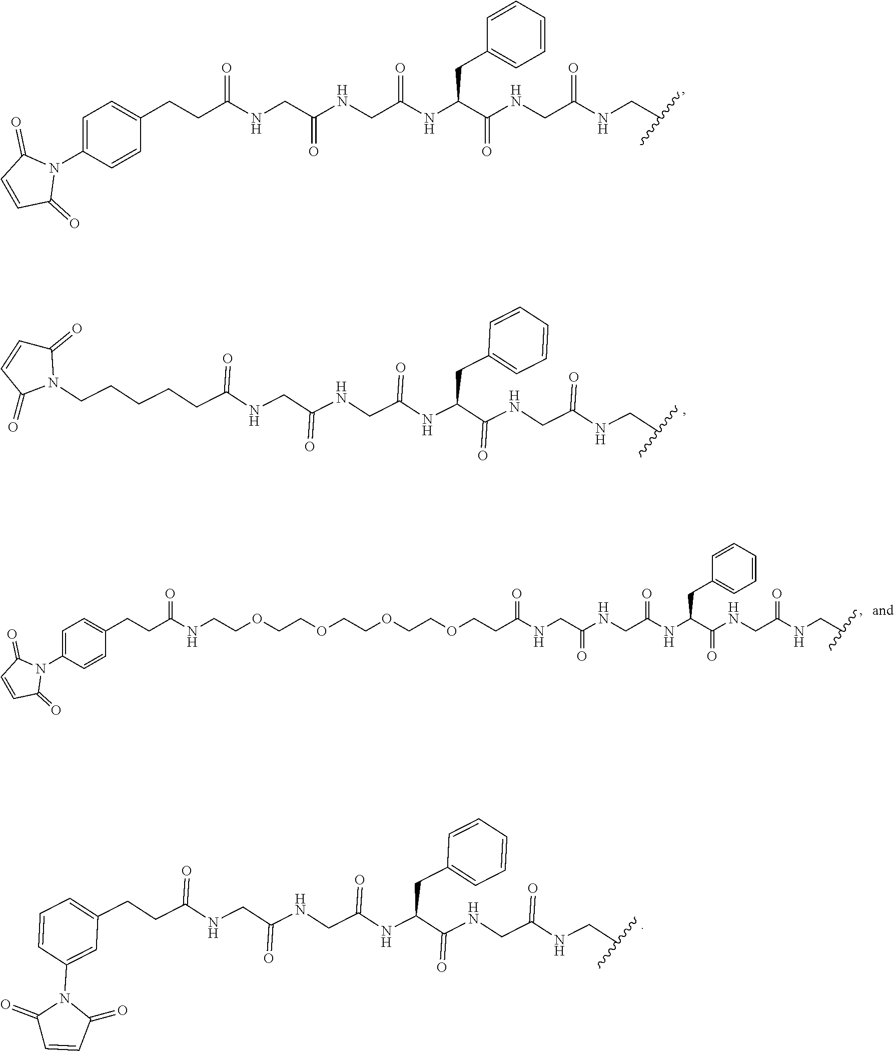 [SMILES: *CNC(=O)CNC(=O)[C@H](Cc1ccccc1)NC(=O)CNC(=O)CNC(=O)CCc1ccc(N2C(=O)C=CC2=O)cc1 ; *CNC(=O)CNC(=O)[C@H](Cc1ccccc1)NC(=O)CNC(=O)CNC(=O)CCCCCN1C(=O)C=CC1=O ; *CNC(=O)CNC(=O)[C@H](Cc1ccccc1)NC(=O)CNC(=O)CNC(=O)CCOCCOCCOCCOCCNC(=O)CCc1ccc(N2C(=O)C=CC2=O)cc1 ; *CNC(=O)CNC(=O)[C@H](Cc1ccccc1)NC(=O)CNC(=O)CNC(=O)CCc1cccc(N2C(=O)C=CC2=O)c1]

In an aspect, the present disclosure provides a compound of Formula (IV):

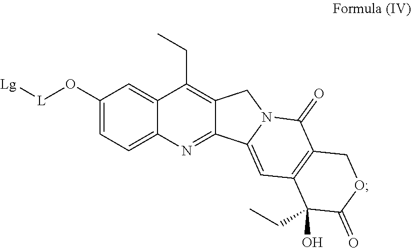

or a pharmaceutically acceptable salt thereof, wherein;
Lg is a ligand;
L is L²-L³-L⁴-(L⁵)ₘ-(L⁶)ₙ-(L⁷)ₚ-R²;
L² is selected from a C₁₋₆ alkylene;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(—NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocycle;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
m is selected from 0 and 1;
n is selected from 0 and 1;
p is selected from 0 and 1;
q is selected from 0 to 8; and
s is selected from 0 and 1.

In some embodiments, Formula (IV)* is represented by

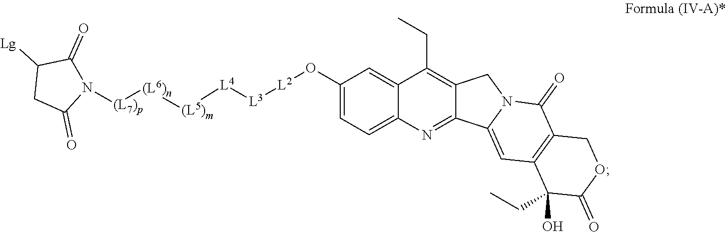

or a pharmaceutically acceptable salt thereof.

Included in the present disclosure are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

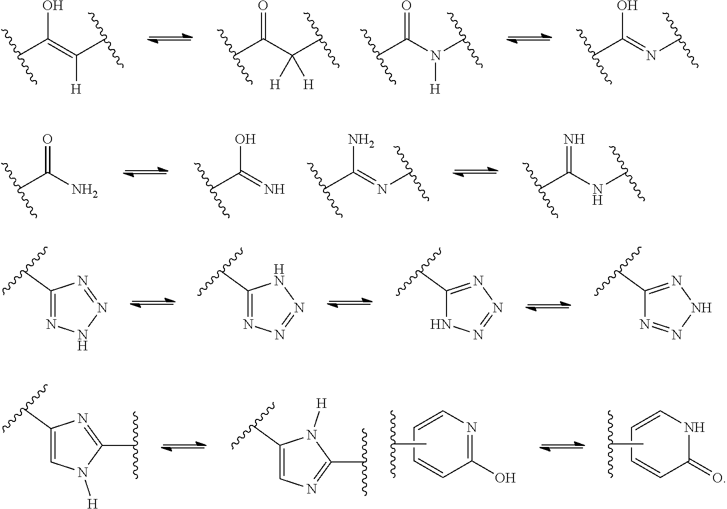

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Ligands/Antibodies

In some embodiments, for a compound or salt of Formula (I), Formula (I-A), Formula (I-B), Formula (II), Formula (III), Formula (III-A), Formula (V-A), Formula (V-B), Formula (V-C), Formula (V-D), Formula (III)*, Formula (III-A), Formula (IV)*, or Formula (IV-A)* the ligand is selected from an antibody or an antigen-binding fragment thereof. In some cases, the ligand is selected from a group consisting of chimeric antibodies, humanized antibodies, and human antibodies.

In some embodiments, the ligand (e.g., antibody), performs a targeting function. By binding to a target tissue or cell where its antigen or receptor is located, the ligand directs the conjugate there. In some cases, when the ligand is an antibody, the compound is sometimes referred to as antibody-drug conjugate (ADC) or an immunoconjugate. Preferably, the target tissue or cell is a cancer tissue or cell, and the antigen or receptor is a tumor-associated antigen, that is, an antigen that is uniquely expressed by cancerous cells or is overexpressed by cancer cells, compared to non-cancerous cells. In some instances, the conjugate is internalized into a target cell by endocytosis and cleavage takes place within the target cell.

In some embodiments, the antibody or antigen-binding fragment thereof binds an antigen selected from the group comprising or consisting of CD19, CD20, CD22, CD30, CD37, CD79b, HER2, and PSMA. In some embodiments, the antibody or antigen-binding fragment thereof binds to HER2. In some cases, the antibody or antigen-binding fragment thereof is selected from trastuzumab, brentuximab, loncastuximab, rosopatamab, rituximab, pinatuzumab, polatuzumab, and naratuximab. In some cases, the antibody is trastuzumab.

In some embodiments, the ligand is an antibody against a tumor associated antigen, allowing the selective targeting of cancer cells. Examples of such antigens include: mesothelin, prostate specific membrane antigen (PSMA), CD19, CD22, CD30, CD70, B7H3, B7H4 (also known as 08E), protein tyrosine kinase 7 (PTK7), glypican-3, RG1, fucosyl-GM1, CTLA-4, and CD44. The antibody can be animal (e.g., murine), chimeric, humanized, or, preferably, human. The antibody preferably is monoclonal, especially a monoclonal human antibody. The preparation of human monoclonal antibodies against some of the aforementioned antigens is disclosed in Korman et al., U.S. Pat. No. 8,609,816 B2 (2013; B7H4, also known as 08E; in particular antibodies 2A7, 1G11, and 2F9); Rao-Naik et al., U.S. Pat. No. 8,097,703 B2 (2012; CD19; in particular antibodies 5G7, 13F1, 46E8, 21D4, 21D4a, 47G4, 27F3, and 3C10); King et al., U.S. Pat. No. 8,481,683 B2 (2013; CD22; in particular antibodies 12C5, 19A3, 16F7, and 23C6); Keler et al., U.S. Pat. No. 7,387,776 B2 (2008; CD30; in particular antibodies 5F11, 2H9, and 17G1); Terrett et al., U.S. Pat. No. 8,124,738 B2 (2012; CD70; in particular antibodies 2H5, 10B4, 8B5, 18E7, and 69A7); Korman et al., U.S. Pat. No. 6,984,720 B1 (2006; CTLA-4; in particular antibodies 10D1, 4B6, and 1E2); Vistica et al., U.S. Pat. No. 8,383,118 B2 (2013, fucosyl-GM1, in particular antibodies 5B1, 5B1a, 7D4, 7E4, 13B8, and 18D5) Korman et al., U.S. Pat. No. 8,008,449 B2 (2011; PD-1; in particular antibodies 17D8, 2D3, 4H1, 5C4, 4A11, 7D3, and 5F4); Huang et al., US 2009/0297438 A1 (2009; PSMA. in particular antibodies 1C3, 2A10, 2F5, 2C6); Cardarelli et al., U.S. Pat. No. 7,875,278 B2 (2011; PSMA; in particular antibodies 4A3, 7F12, 8C12, 8A11, 16F9, 2A10, 2C6, 2F5, and 1C3); Terrett et al., U.S. Pat. No. 8,222,375 B2 (2012; PTK7; in particular antibodies 3G8, 4D5, 12C6, 12C6a, and 7C8); Terrett et al., U.S. Pat. No. 8,680,2471B2 (2014; glypican-3; in particular antibodies 4A6, 11E7, and 16D10); Harkins et al., U.S. Pat. No. 7,335,748 B2(2008; RG1; in particular antibodies A, B, C, and D); Terrett et al., U.S. Pat. No. 8,268,970 B2 (2012; mesothelin; in particular antibodies 3C10, 6A4, and 7B1); Xu et al., US 2010/0092484 A1 (2010; CD44; in particular antibodies 14G9.B8.B4, 2D1.A3.D12, and 1A9.A6.B9); Deshpande et al., U.S. Pat. No. 8,258,2661B2 (2012; IP10; in particular antibodies 1D4, 1E1, 2G1, 3C4, 6A5, 6A8, 7C10, 8F6, 10A12, 10A2S, and 13C4); Kuhne et al., U.S. Pat. No. 8,450,464 B2 (2013; CXCR4; in particular antibodies F7, F9, D1, and E2); and Korman et al., U.S. Pat. No. 7,943,743 B2 (2011; PD-L1; in particular antibodies 3G10, 12A4, 10A5, 5F8, 10H10, 1B12, 7H1, 11E6, 12B7, and 13G4); the disclosures of which are incorporated herein by reference.

In some embodiments, the ligand can also be an antibody fragment or antibody mimetic, such as an affibody, a domain antibody (dAb), a nanobody, a unibody, a DARPin, an anticalin, a versabody, a duocalin, a lipocalin, or an avimer.

In some embodiments, any one of several different reactive groups on the ligand can be a conjugation site, including ε-amino groups in lysine residues, pendant carbohydrate moieties, carboxylic acid groups, disulfide groups, and thiol groups. Each type of reactive group represents a trade-off, having some advantages and some disadvantages. For reviews on antibody reactive groups suitable for conjugation, see, e.g., Garnett, Adv. Drug Delivery Rev. 53 (2001), 171-216 and Dubowchik and Walker, Pharmacology & Therapeutics 83 (1999), 67-123, the disclosures of which are incorporated herein by reference.

In some embodiments, the ligand is conjugated via a lysine F-amino group. Most antibodies have multiple lysine F-amino groups, which can be conjugated via amide, urea, thiourea, or carbamate bonds using techniques known in the art. However, it is difficult to control which and how many F-amino groups react, leading to potential batch-to-batch variability in conjugate preparations. Also, conjugation may cause neutralization of a protonated F-amino group important for maintaining the antibody's native conformation or may take place at a lysine near or at the antigen binding site, neither being a desirable occurrence.

In some embodiments, the ligand can be conjugated via a carbohydrate side chain, as many antibodies are glycosylated. The carbohydrate side chain can be oxidized with periodate to generate aldehyde groups, which in turn can be reacted with amines to form an imine group, such as in a semicarbazone, oxime, or hydrazone. If desired, the imine group can be converted to a more stable amine group by reduction with sodium cyanoborohydride. For additional disclosures on conjugation via carbohydrate side chains, see, e.g., Rodwell et al., Proc. Nat'l Acad. Sci. USA 83, 2632-2636 (1986); the disclosure of which is incorporated herein by reference. As with lysine F-amino groups, there are concerns regarding reproducibility of the location of the conjugation site(s) and stoichiometry.

In some embodiments, the ligand can be conjugated via a carboxylic acid group. In some cases, a terminal carboxylic acid group is functionalized to generate a carbohydrazide, which is then reacted with an aldehyde-bearing conjugation moiety. See Fisch et al., Bioconjugate Chemistry 1992, 3, 147-153.

In some embodiments, the antibody can be conjugated via a disulfide group bridging a cysteine residue on the antibody and a sulfur on the other portion of the conjugate or compound. Some antibodies lack free thiol (sulfhydryl) groups but have disulfide groups, for example in the hinge region. In such case, free thiol groups can be generated by reduction of native disulfide groups. The thiol groups so generated can then be used for conjugation. See, e.g., Packard et al., Biochemistry 1986, 25, 3548-3552; King et al., Cancer Res. 54, 6176-6185 (1994); and Doronina et al., Nature Biotechnol. 21(7), 778-784 (2003); the disclosures of which are incorporated herein by reference. A number of methods are known for introducing free thiol groups into antibodies without breaking native disulfide bonds, which methods can be practiced with the ligand of this invention. Depending on the method employed, it may be possible to introduce a predictable number of free sulfhydryls at predetermined locations. In one approach, mutated antibodies are prepared in which a cysteine is substituted for another amino acid. See, for example, Eigenbrot et al., U.S. Pat. No. 7,521,541 B2 (2009); Chilkoti et al., Bioconjugate Chem. 1994, 5, 504-507; Urnovitz et al., U.S. Pat. No. 4,698,420 (1987); Stimmel et al., J. Biol. Chem., 275 (39), 30445-30450 (2000); Bam et al., U.S. Pat. No. 7,311,902 B2 (2007); Kuan et al., J. Biol. Chem., 269 (10), 7610-7618 (1994); Poon et al., J. Biol. Chem., 270 (15), 8571-8577 (1995). In another approach, an extra cysteine is added to the C-terminus. See, e.g. Cumber et al., J. Immunol., 149, 120-126 (1992); King et al, Cancer Res., 54, 6176-6185 (1994); Li et al., Bioconjugate Chem., 13, 985-995 (2002); Yang et al., Protein Engineering, 16, 761-770 (2003); and Olafson et al., Protein Engineering Design & Selection, 17, 21-27 (2004). A preferred method for introducing free cysteines is that taught by Liu et al., WO 2009/026274 A1, in which a cysteine bearing amino acid sequence is added to the C-terminus of the heavy chain of an antibody. This method introduces a known number of cysteine residues (one per heavy chain) at a known location away from the antigen binding site. The disclosures of the documents cited in this paragraph are all incorporated herein by reference.

In some embodiments, the lysine &-amino groups can be modified with reagents such as 2-iminothiolane or N-succinimidyl-3-(2-pyridyldithio)propionate (SPDP), converting an &-amino group into a thiol or disulfide group—creating a cysteine surrogate, as it were.

### Linkers

The compounds and salts of Formula (I), Formula (I)*, Formula (I-A), Formula (I-B), Formula (II), Formula (II)*, Formula (III), Formula (III)*, Formula (IV)*, or Formula (IV-A)* may contain a linker (e.g., L). The linker may be as described elsewhere herein. In some cases, L is a peptide linker. In some embodiments, the linker is also bound to a ligand (e.g., antibody) and referred to as an antibody drug conjugate or conjugate. Linkers of the conjugates described herein may not affect the binding of active portions of a conjugate, e.g., the antigen binding domains, Fc domains, target binding domains, antibodies, agonists or the like, to a target, which can be a cognate binding partner such as an antigen. A conjugate can comprise multiple linkers, each having one or more compounds attached. These linkers can be the same linkers or different linkers.

In some embodiments, a linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker may comprise a valine-citrulline linker or a valine-alanine peptide. A valine-citrulline- or valine-alanine-containing linker can contain a maleimide or succinimide group.

In some embodiments, a non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly) linker. A linker can be a linker suitable for attachment to an engineered cysteine (THIOMAB). A THIOMAB linker can be a (maleimidocaproyl)-(valine-citrulline)-(para-aminobenzyloxycarbonly)- linker.

In some embodiments, a linker can also comprise alkylene, alkenylene, alkynylene, polyether, polyester, polyamide group(s) and also, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif to an N-terminal GGG motif to regenerate a native amide bond. The linker created can therefore link a moiety attached to the LXPTG recognition motif with a moiety attached to the N-terminal GGG motif.

In some embodiments, a compound or salt of any one of Formulas described herein is linked to the antibody construct by way of a linker(s), also referred to herein as L or linker. L, as used herein, may be selected from any of the linker moieties discussed herein. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody construct, or monovalent such that covalently they link a single compound or salt to a single site on the antibody construct.

In some embodiments, linkers may have from about 10 to about 500 atoms in a linker, such as from about 10 to about 400 atoms, such as about 10 to about 300 atoms in a linker. In some embodiments, linkers may have from about 30 to about 400 atoms, such as from about 30 to about 300 atoms in the linker.

In some embodiments, the linkers described herein may link a compound or salt of any one of the Formulas described herein to a ligand (e.g., antibody) by a covalent linkages between the linker and the antibody construct and compound. The linker may include a functional group capable of covalently linking to a ligand (e.g., antibody).

In some embodiments, by way of example and not limitation, some cleavable and noncleavable linkers that may be included in the conjugates described herein are described below.

In some embodiments, cleavable linkers can be cleavable in vitro and in vivo. Cleavable linkers can include chemically or enzymatically unstable or degradable linkages. Cleavable linkers can rely on processes inside the cell to liberate a benzazepine compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

In some embodiments, a linker can contain a chemically labile group such as hydrazone and/or disulfide groups. Linkers comprising chemically labile groups can exploit differential properties between the plasma and some cytoplasmic compartments. Cleavable linkers can also include a disulfide group.

In some embodiments, acid-labile groups, such as hydrazone, can remain intact during systemic circulation in the blood's neutral pH environment (pH 7.3-7.5) and can undergo hydrolysis and can release the benzazepine compound once the antibody construct benzazepine compound conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation. Hydrazone-containing linkers can contain additional cleavage sites, such as additional acid-labile cleavage sites and/or enzymatically labile cleavage sites. Other acid-labile groups that can be included in linkers include cis-aconityl-containing linkers. cis-Aconityl chemistry can use a carboxylic acid juxtaposed to an amide bond to accelerate amide hydrolysis under acidic conditions.

In some embodiments, the linker may be specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers. Peptide bonds can have good serum stability, as lysosomal proteolytic enzymes can have very low activity in blood due to endogenous inhibitors and the unfavorably high pH value of blood compared to lysosomes. Release of a compound described herein from an antibody drug conjugate can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, cathepsin S, β-glucuronidase, or β-galactosidase. The cleavable peptide can be selected from tetrapeptides such as Gly-Phe-Leu-Gly, Ala-Leu-Ala-Leu or dipeptides such as Val-Cit, Val-Ala, and Phe-Lys. Dipeptides can have lower hydrophobicity compared to longer peptides. A variety of dipeptide-based cleavable linkers can be used in the antibody drug conjugates described herein. The enzymatically cleavable linker can be a 13-glucuronic acid-based linker.

In some embodiments, cleavable linkers can include non-cleavable portions or segments, and/or cleavable segments or portions can be included in an otherwise non-cleavable linker to render it cleavable.

In some embodiments, a linker can contain an enzymatically cleavable peptide moiety. The peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

### Pharmaceutical Formulations

Provided herein, in certain embodiments, are compositions comprising a therapeutically effective amount of any compound or salt of any one of Formula (I), Formula (I)*, Formula (I-A), Formula (I-B), Formula (II), Formula (II)*, Formula (III), Formula (III)*, Formula (IV), Formula (IV)*, Formula (IV-A)*, Formula (V-A), Formula (V-B), Formula (V-C), or Formula (V-D), (also referred to herein as “a pharmaceutical agent”).

Pharmaceutical compositions may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa, e.g., sublingually; anally, rectally or vaginally, for example, as a pessary, cream or foam; parenterally, including intramuscularly, intravenously, subcutaneously or intrathecally as, for example, a sterile solution or suspension; nasally; intraperitoneally; subcutaneously; transdermally, for example, as a patch applied to the skin; and topically, for example, as a cream, ointment or spray applied to the skin, or as an eye drop. The compound may also be formulated for inhalation. In certain embodiments, a compound may be simply dissolved or suspended in sterile water.

A pharmaceutical composition may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

Subjects may generally be monitored for therapeutic effectiveness using assays and methods suitable for the condition being treated, which assays will be familiar to those having ordinary skill in the art and are described herein. Pharmacokinetics of a pharmaceutical agent, or one or more metabolites thereof, that is administered to a subject may be monitored by determining the level of the pharmaceutical agent or metabolite in a biological fluid, for example, in the blood, blood fraction, e.g., serum, and/or in the urine, and/or other biological sample or biological tissue from the subject. Any method practiced in the art and described herein to detect the agent may be used to measure the level of the pharmaceutical agent or metabolite during a treatment course.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipients selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and in the art.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical agents described herein can be used alone or in combination with appropriate additives to make tablets, powders, granules or capsules, and if desired, with diluents, buffering agents, moistening agents, preservatives, coloring agents, and flavoring agents. The pharmaceutical agents may be formulated with a buffering agent to provide for protection of the compound from low pH of the gastric environment and/or an enteric coating. A pharmaceutical agent included in a pharmaceutical composition may be formulated for oral delivery with a flavoring agent, e.g., in a liquid, solid or semi-solid formulation and/or with an enteric coating.

A pharmaceutical composition comprising any one of the pharmaceutical agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of pharmaceutical agent contained within a sustained release formulation depends upon the site of implantation, the rate and expected duration of release, and the nature of the condition, disease or disorder to be treated or prevented.

In certain embodiments, the pharmaceutical compositions comprising a pharmaceutical agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions comprising a pharmaceutical agent can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

In some embodiments, the pharmaceutical agent described herein can be formulated as in inhalant. Inhaled methods can deliver medication directly to the airway. The pharmaceutical agent can be formulated as aerosols, microspheres, liposomes, or nanoparticles. The pharmaceutical agent can be formulated with solvents, gases, nitrates, or any combinations thereof. Compositions described herein are optionally formulated for delivery as a liquid aerosol or inhalable dry powder. Liquid aerosol formulations are optionally nebulized predominantly into particle sizes that can be delivered to the terminal and respiratory bronchioles. Liquid aerosol and inhalable dry powder formulations are preferably delivered throughout the endobronchial tree to the terminal bronchioles and eventually to the parenchymal tissue.

Aerosolized formulations described herein are optionally delivered using an aerosol forming device, such as a jet, vibrating porous plate or ultrasonic nebulizer, preferably selected to allow the formation of aerosol particles having with a mass medium average diameter predominantly between 1 to 5μ. Further, the formulation preferably has balanced osmolarity ionic strength and chloride concentration, and the smallest aerosolizable volume able to deliver effective dose of the pharmaceutical agent. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5μ. Predominantly in this application means that at least 70% but preferably more than 90% of all generated aerosol particles are within 1-5μ range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, California), Sidestream® nebulizers (Medic-Aid Ltd., West Sussex, England), Pari LC® and Pari LC Star® jet nebulizers (Pari Respiratory Equipment, Inc., Richmond, Virginia), and Aerosonic™ (DeVilbiss Medizinische Produkte (Deutschland) GmbH, Heiden, Germany) and UltraAire® (Omron Healthcare, Inc., Vernon Hills, Illinois) ultrasonic nebulizers.

In some embodiments, the pharmaceutical agent(s) can be formulated with oleaginous bases or ointments to form a semisolid composition with a desired shape. In addition to the pharmaceutical agent, these semisolid compositions can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

Controlled or sustained release transdermal or topical formulations can be achieved by the addition of time-release additives, such as polymeric structures, matrices, that are available in the art. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

An insert, transdermal patch, bandage or article can comprise a mixture or coating of polymers that provide release of the pharmaceutical agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

Transdermal devices (inserts, patches, bandages) may also comprise a water insoluble polymer. Rate controlling polymers may be useful for administration to sites where pH change can be used to effect release. These rate controlling polymers can be applied using a continuous coating film during the process of spraying and drying with the active compound. In one embodiment, the coating formulation is used to coat pellets comprising the active ingredients that are compressed to form a solid, biodegradable insert.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the pharmaceutical agent. The matrices can be formed by solvent evaporation, spray drying, solvent extraction and other methods known to those skilled in the art.

Kits with unit doses of one or more of the agents described herein, usually in oral or injectable doses, are provided. Such kits may include a container containing the unit dose, an informational package insert describing the use and attendant benefits of the drugs in treating disease, and optionally an appliance or device for delivery of the composition.

### Methods of Treatment

In an aspect, the present disclosure provides a method of treating a subject with a tumor. In some cases, the treatment of a subject with the tumor includes administering to a subject in need thereof a compound or salt of any one of Formula (I), Formula (I)*, Formula (I-A), Formula (I-B), Formula (II), Formula (II)*, Formula (III), Formula (III)*, Formula (IV), Formula (IV)*, or Formula (IV-A)*, Formula (V-A), Formula (V-B), Formula (V-C), Formula (V-D), or a pharmaceutical composition of any one thereof. In some cases, the tumor is associated with a cancer. In some cases, the cancer is selected from the group consisting of lung cancer, kidney cancer, urethral cancer, colorectal cancer, prostate cancer, glioblastoma multiforme, ovarian cancer, pancreatic, breast, melanoma, liver, bladder, stomach, and esophageal cancers.

In some embodiments, compounds described herein can be used for treating diseases or disorders, such as, but not limited to, hyperproliferative diseases, including: cancers of the head and neck which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; ovarian cancer; small cell and non-small cell lung cancer (SCLC and NSCLC); breast cancer sarcomas, such as fibrosarcoma, malignant fibrous histiocytoma, embryonal rhabdomyosarcoma, leiomysosarcoma, neurofibrosarcoma, osteosarcoma, synovial sarcoma, liposarcoma, and alveolar soft part sarcoma; leukemias such as acute promyelocytic leukemia (APL), acute myelogenous leukemia (AML), acute lymphoblastic leukemia (ALL), and chronic myelogenous leukemia (CML); neoplasms of the central nervous systems, particularly brain cancer; multiple myeloma (MM), lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma. Clinically, practice of the methods and use of compositions described herein will result in a reduction in the size or number of the cancerous growth and/or a reduction in associated symptoms (where applicable). Pathologically, practice of the method and use of compositions described herein will produce a pathologically relevant response, such as: inhibition of cancer cell proliferation, reduction in the size of the cancer or tumor, prevention of further metastasis, and inhibition of tumor angiogenesis. The method of treating such diseases comprises administering a therapeutically effective amount of an inventive combination to a subject. The method may be repeated as necessary. The cancer can be renal, lung, gastric, or ovarian cancer. In some cases, the cancer includes malignancies of the various organ systems, such as affecting skin, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the oesophages.

In some embodiments, the treatment of tumor-bearing subjects inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal.

In some embodiments, compounds described herein can be administered in combination with other therapeutic agents, including antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, β-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, and vindesine.

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

In certain embodiments, the invention provides a method of treating or preventing a disease, state, or condition in a patient in need thereof comprising administering to the patient an effective amount of a compound of any one of embodiments of the invention or a pharmaceutically acceptable salt thereof. The disease, state or condition may be selected from a group as described elsewhere herein.

### Preparation of Compounds

The compounds of the present disclosure can generally be prepared in a number of ways well known to those skilled in the art of organic synthesis. By way of example, compounds of the present disclosure can be synthesized using the methods described herein, together with synthetic methods known in the art of synthetic organic chemistry, or variations thereof as appreciated by those skilled in the art. The compounds of the present disclosure may be prepared as described in the schemes and examples described elsewhere herein.

The following examples further illustrate the invention but, of course, should not be construed as in any way limiting its scope.

### EXAMPLES

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein. As used below, and throughout the description of the invention, the following abbreviations, unless otherwise indicated, shall be understood to have the following meanings:

| | |
|---|---|
| ACN | acetonitrile |
| °C | degrees Celsius |
| DBU | 1,8-Diazabicyclo[5.4.0]undec-7-ene |
| DCM | dichloromethane (CH₂Cl₂) |
| DIEA | diisopropylethylamine |
| DMF | dimethylformamide |
| DMA | dimethylacetamide |
| DMSO | dimethylsulfoxide |
| ESI | electrospray ionization |
| EDTA | Ethylenediaminetetraacetic acid |
| g | gram(s) |
| h | (hours) |
| HOAt | 1-Hydroxy-7-azabenzotriazole |
| HPLC | high performance liquid chromatography |
| LCMS | liquid chromatography mass spectrometry |
| μ | micro |
| M | molar |
| M⁺ | parent molecular ion |
| min | minute(s) |
| mol | mole(s); molecular (as in mol wt) |
| mL | milliliter |
| MS | mass spectrometry |
| NAC | N-acetyl cysteine |
| nm | nanometer(s) |
| NMR | nuclear magnetic resonance |
| PBS | Phosphate-buffered saline |
| PES | Polyethersulfone |
| pH | potential of hydrogen; a measure of the acidity |
| | or basicity of an aqueous |
| | solution |
| PE | petroleum ether |
| PyAOP | (7-Azabenzotriazol-1-yloxy)tripyrrolidinophosphonium |
| hexa- | |
| fluorophosphate | |
| RT | room temperature |
| TFA | trifluoroacetic acid |
| Tris | Tris(hydroxymethyl)aminomethane |
| TCEP | tris(2-carboxyethyl)phosphine |

### Preparation of Drug Linkers

### Example 1: Synthesis of Compound 11

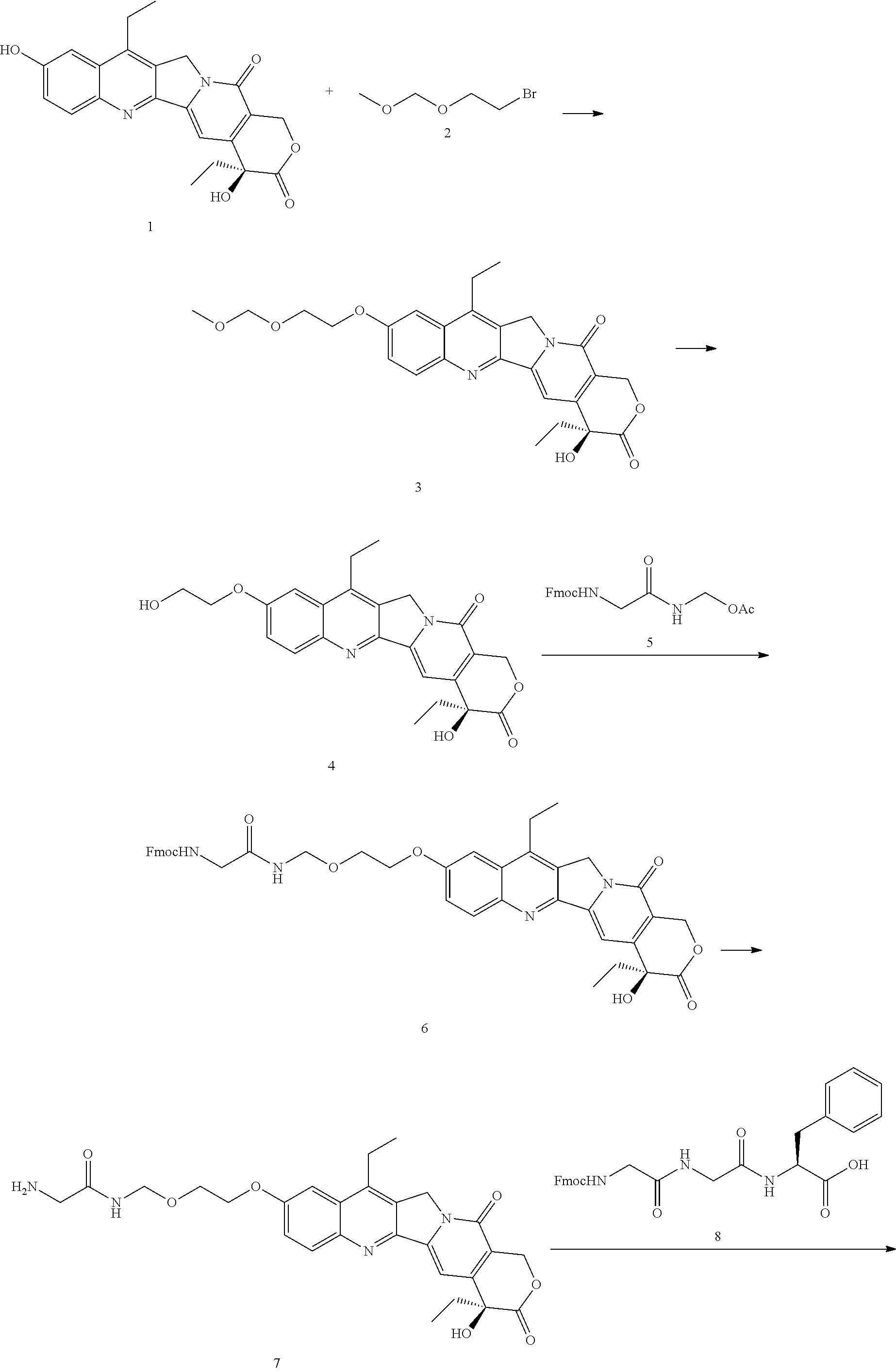

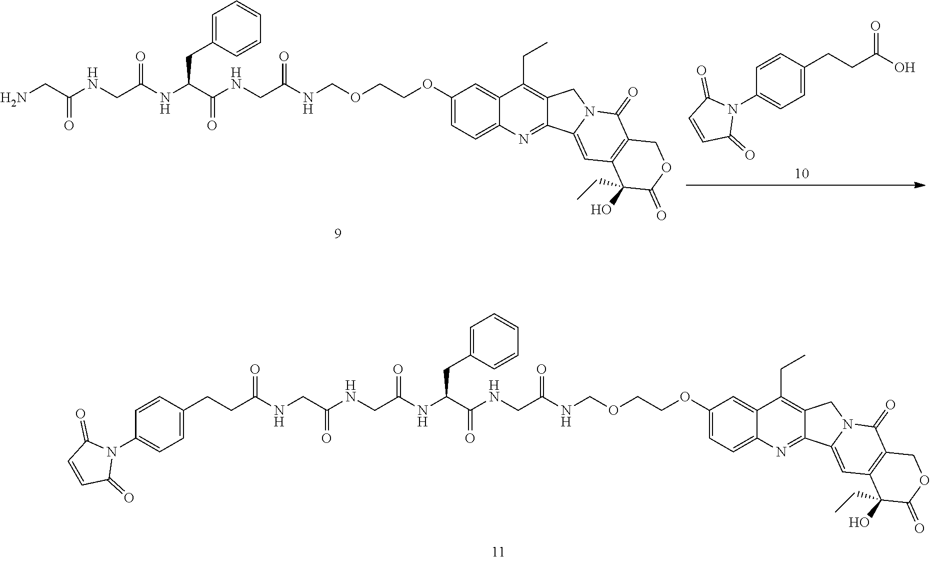 [SMILES: CCc1c2c(nc3ccc(OCCOCNC(=O)CNC(=O)[C@H](Cc4ccccc4)NC(=O)CNC(=O)CN)cc13)-c1cc3c(c(=O)n1C2)COC(=O)[C@]3(O)CC ; O=C(O)CCc1ccc(N2C(=O)C=CC2=O)cc1 ; CCc1c2c(nc3ccc(OCCOCNC(=O)CNC(=O)[C@H](Cc4ccccc4)NC(=O)CNC(=O)CNC(=O)CCc4ccc(N5C(=O)C=CC5=O)cc4)cc13)-c1cc3c(c(=O)n1C2)COC(=O)[C@]3(O)CC]

Compound 3: To a solution of SN-38 (Compound 1, 1.57 g, 4 mmol) in DMF (20 mL) was added compound 2 (1.4 g, 8 mmol) and DBU (0.6 g, 4 mmol). The reaction mixture was stirred at 50 °C for 2 h, and then purified directly by RP-HPLC to give compound 3 as a yellow solid (0.42 g) after lyophilization.

Compound 4: Compound 3 (0.4 g) was suspended in DCM (6 mL) and TFA (4 mL) was added. The mixture was stirred at room temperature for 2 h. Then the reaction was concentrated to dryness under reduced pressure. The residue was purified by RP-HPLC to give compound 4 as a yellow solid (238 mg) after lyophilization.

Compound 6: To a solution of compound 4 (204 mg, 0.47 mmol) in a mixture of DCM (4 mL) and TFA (1 mL) was added compound 5 (860 mg, 2.34 mmol). The mixture was stirred at room temperature for a period of 1 h. Then the solvent was removed under vacuum and the mixture was purified by RP-HPLC. The fractions were lyophilized to give compound 6 (271 mg) as a pale yellow solid.

Compound 7: To a solution of compound 6 (271 mg, 0.36 mmol) in DMF (2 mL) was added piperidine (0.2 mL). The mixture was stirred at room temperature for 10 min, then purified by RP-HPLC. The fractions were lyophilized to give compound 7 (191 mg) as a pale yellow solid.

Compound 9: To a solution of compound 7 (TFA salt, 114 mg, 0.18 mmol) in DMF (2 mL) was added compound 8 (92 mg, 0.18 mmol), DIEA (128 μL, 0.74 mmol), and PyAOP (96 mg, 0.18 mmol). The mixture was stirred at room temperature for 10 min and then piperidine (0.2 mL) was added. The stirring continued for an additional 10 min, then the mixture was purified by RP-HPLC. The fractions were lyophilized to give compound 9 (84 mg) as a pale yellow solid.

Compound 11: To a solution of compound 10 (4.1 mg, 16.7 μmol) in DMF (1 mL) was added HATU (6.4 mg, 16.7 μmol) and DIEA (8.6 μL, 50 μmol). The mixture was stirred at room temperature for 1 min and then compound 9 (TFA salt, 15 mg, 16.7 μmol) was added. The stirring continued for an additional 10 min, then the mixture was purified by RP-HPLC. The fractions were lyophilized to give compound 11 (12 mg) as a pale yellow solid. MS: 1011.8 [M+H]⁺.

### Example 2: Synthesis of Compound 13

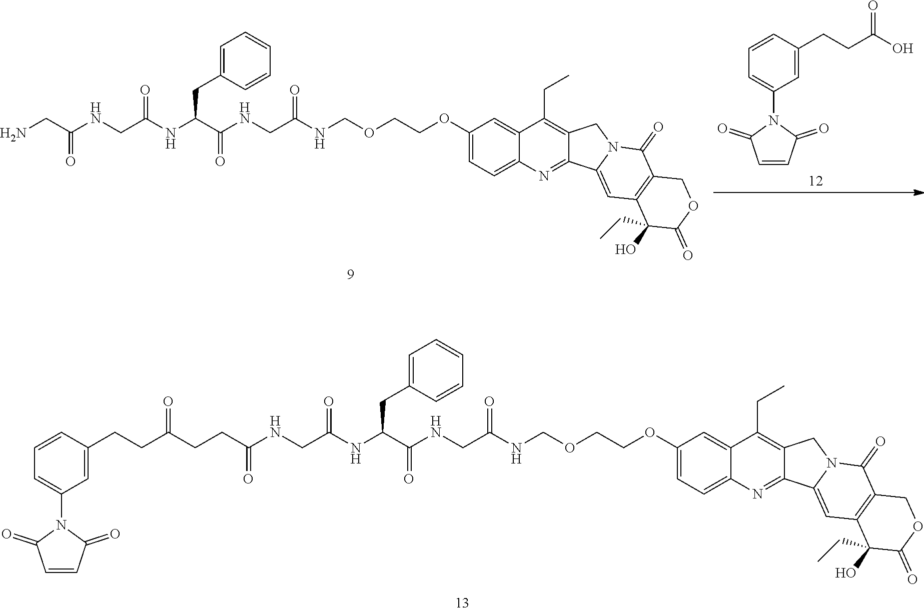 [SMILES: CCc1c2c(nc3ccc(OCCOCNC(=O)CNC(=O)[C@H](Cc4ccccc4)NC(=O)CNC(=O)CN)cc13)-c1cc3c(c(=O)n1C2)COC(=O)[C@]3(O)CC ; O=C(O)CCc1cccc(N2C(=O)C=CC2=O)c1 ; CCc1c2c(nc3ccc(OCCOCNC(=O)CNC(=O)[C@H](Cc4ccccc4)NC(=O)CNC(=O)CCC(=O)CCc4cccc(N5C(=O)C=CC5=O)c4)cc13)-c1cc3c(c(=O)n1C2)COC(=O)[C@]3(O)CC]

To a solution of compound 12 (4.1 mg, 16.7 mol) in DMF (1 mL) was added HATU (6.4 mg, 16.7 μmol) and DIEA (8.6 μL, 50 μmol). The mixture was stirred at room temperature for 1 min and then compound 9 (TFA salt, 15 mg, 16.7 μmol) was added. The stirring continued for an additional 10 min, then the mixture was purified by RP-HPLC. The fractions were lyophilized to give compound 13 (12 mg) as a pale-yellow solid. MS: 1011.4 [M+H]⁺.

### Example 3: Synthesis of Compound 15

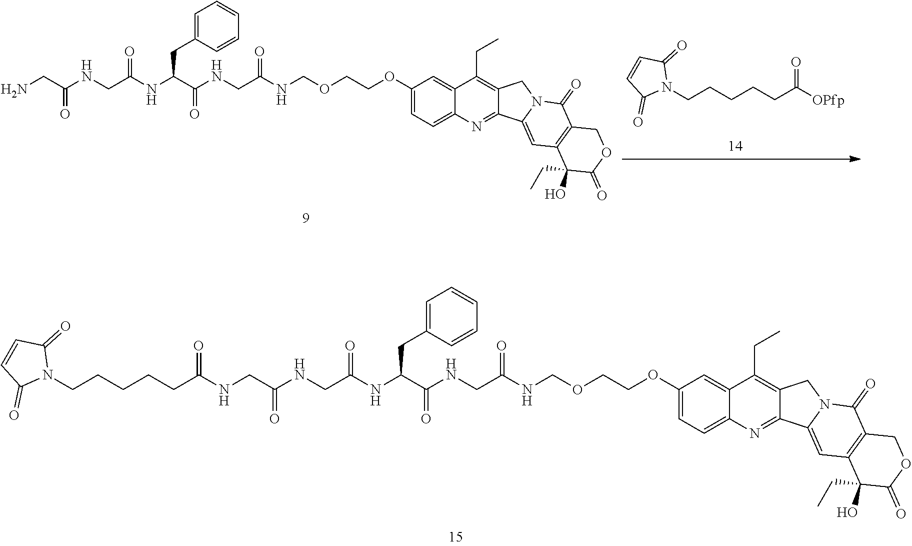

To a solution of compound 9 (TFA salt, 15 mg, 16.7 μmol) in DMF (1 mL) was added compound 14 (6.3 mg, 16.7 μmol) and DIEA (8.6 μL, 50 μmol). The mixture was stirred at room temperature for 10 min, then the mixture was purified by RP-HPLC. The fractions were lyophilized to give compound 15 (13 mg) as a pale yellow solid. MS: 977.4 [M+H]+.

### Example 4: Synthesis of Compound 18

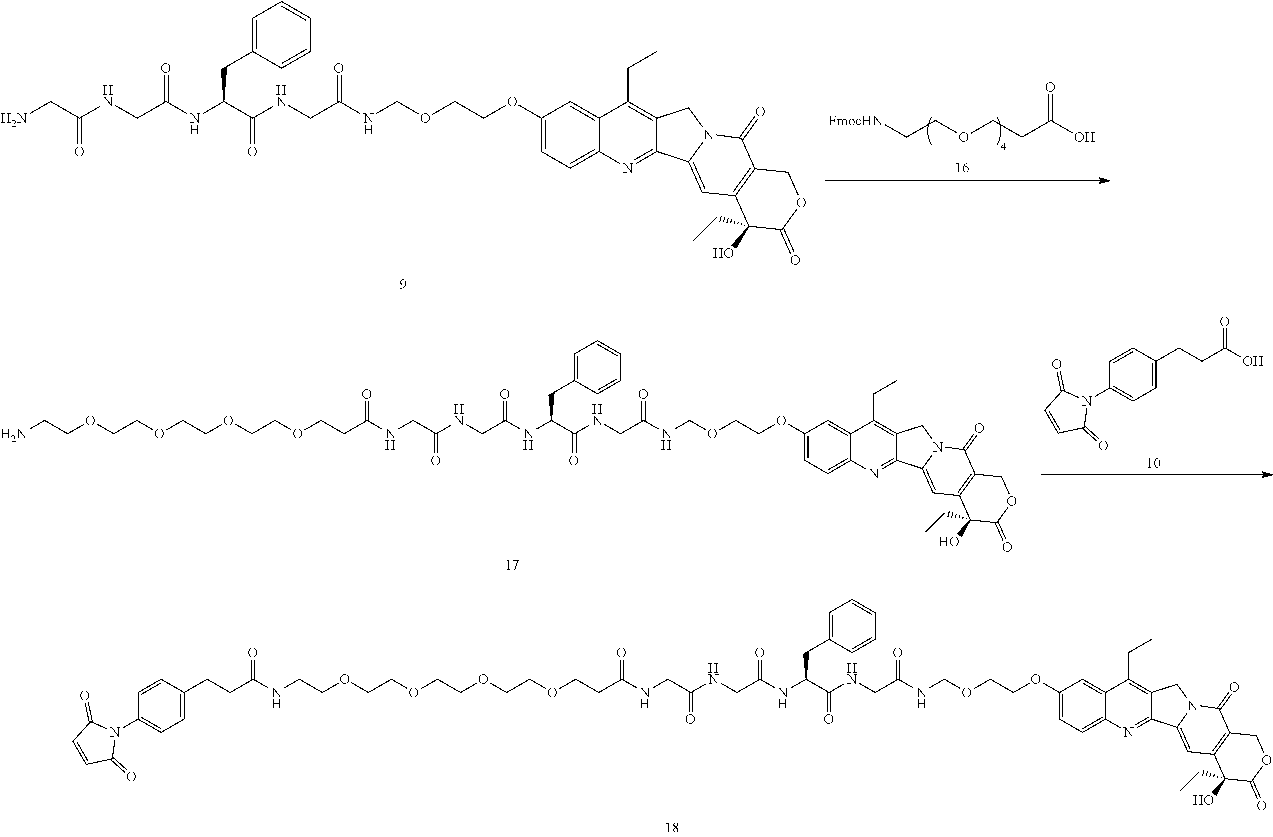

Compound 17: To a solution of compound 16 (10.3 mg, 21.2 μmol) in DMF (1 mL) was added HATU (8.1 mg, 21.2 μmol) and DIEA (15 μL, 86 μmol). The mixture was stirred at room temperature for 1 min and then compound 9 (TFA salt, 19 mg, 21.2 μmol) was added. The mixture was stirred at room temperature for 10 min and then piperidine (0.2 mL) was added. The stirring continued for an additional 10 min, then the mixture was purified by RP-HPLC. The fractions were lyophilized to give compound 17 (21 mg) as a pale-yellow solid.

Compound 18: To a solution of compound 10 (4.1 mg, 16.7 μmol) in DMF (1 mL) was added HATU (6.4 mg, 16.7 μmol) and DIEA (8.6 μL, 50 μmol). The mixture was stirred at room temperature for 1 min and then compound 17 (TFA salt, 19.1 mg, 16.7 μmol) was added. The stirring continued for an additional 10 min, then the mixture was purified by RP-HPLC. The fractions were lyophilized to give compound 18 (13 mg) as a pale-yellow solid. MS: 1258.6 [M+H]⁺.

### Example 5: Synthesis of Compound 6

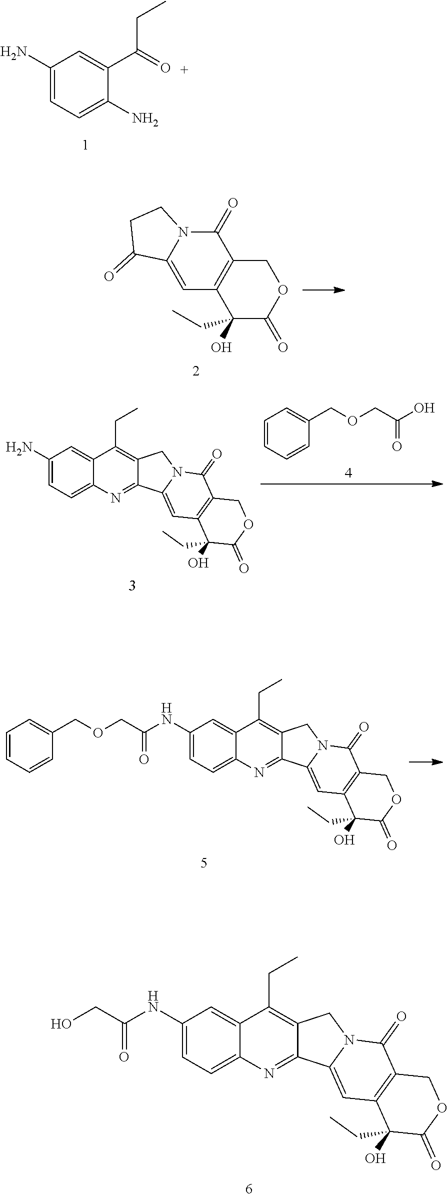 [SMILES: CCC(=O)c1cc(N)ccc1N ; CC[C@@]1(O)C(=O)OCc2c1cc1n(c2=O)CCC1=O ; CCc1c2c(nc3ccc(N)cc13)-c1cc3c(c(=O)n1C2)COC(=O)[C@]3(O)CC ; O=C(O)COCc1ccccc1 ; CCc1c2c(nc3ccc(NC(=O)COCc4ccccc4)cc13)-c1cc3c(c(=O)n1C2)COC(=O)[C@]3(O)CC ; CCc1c2c(nc3ccc(NC(=O)CO)cc13)-c1cc3c(c(=O)n1C2)COC(=O)[C@]3(O)CC]

### Compound 3

A mixture of compound 1 (TFA salt, 150 mg, 0.38 mmol), p-toluenesulfonic acid (36 mg, 0.21 mmol), and compound 2 (100 mg, 0.38 mmol) was refluxed in 20 mL of toluene for 24 h. The solvent was removed under vacuum and the remaining residue was purified by RP-HPLC to give compound 3 (16 mg) as a tan solid.

### Compound 5

To a solution of compound 3 (TFA salt, 7 mg, 13.9 umol) in anhydrous DMF (2 mL) was added acid 4 (2.3 mg, 13.9 umol), PyAOP (7.2 mg, 13.9 umol) and DIEA (10 uL, 55.4 umol). The mixture was stirred at room temperature for a period of 1 h. Then the mixture was purified directly by RP-HPLC to give compound 5 as a tan solid (4 mg).

### Compound 6

Compound 5 (4.0 g, 7.4 umol) was dissolved in ethyl acetate (2 mL), then Pd/C (5 mg) was added and the vessel was filled with hydrogen gas. The mixture was stirred for a period of 1 h; then the catalyst was removed by filtration and the solvent was evaporated under vacuum. The resulting residue was purified by RP-HPLC to give compound 6 as a pale-yellow solid (0.7 mg). MS: 450.1 [M+H]⁺.

### Example 6: Preparation of Antibody-Drug-Conjugates

The conjugates were prepared using the followed conditions using the appropriate Drug-Linker and antibody.

55 mg of antibody in a formulation buffer was pH adjusted or buffer exchanged into 5% (v/v) 0.5 M Tris, 0.025 M EDTA, pH 8.5 formulations for reduction. The addition of 0.025 M EDTA in the formulation buffer was to prevent metal-catalyzed disulphide reoxidation. 10 mM TCEP (7 equivalents per antibody) was added and incubated for 2 h at 20 °C to reduce a target number of interchain disulphide bonds and generate the desired average number of free thiols per antibody. The free thiols were conjugated by adding a 20 mM DMA solution of drug linker (12 equivalents) and stirred for 1 h at 20 °C. Extra DMA solvent was added prior to drug linker addition to maintain solubility of the drug linker upon addition and mixing was performed utilizing stir flasks. Conjugation was ended by the addition of an excess of NAC (10 mM) and further stirred for 30 min to quench unreacted maleimide. Excess quenched drug linker was removed by incubation with activated carbon for 1 h at room temperature at 15 rpm and/or diafiltration. ADC (yield, 91-94%) was exchanged into the final formulation buffer (0.1 M Arg/PBS) using Sephadex G-25 resin column and filtered through 0.2 m PES before aliquoting and storage at ≤−60 °C.

### Example 7: Preparation of Antibody-Drug Conjugate (ADC)-1 Using Drug-Linker 11 and UC-961

ADC-1 was prepared using example 6 and the Drug-Linker 11 and UC-961 antibody.

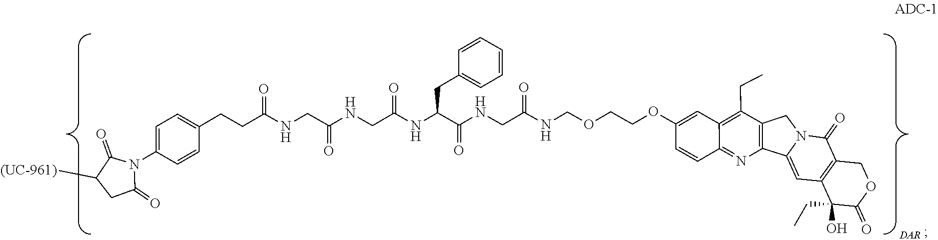

wherein the DAR value as determined by RP-HPLC was 7.8.

### Example 8: Preparation of Antibody-Drug Conjugate (ADC)-2 Using Drug-Linker 13 and UC-961

ADC-2 was prepared using example 6 and the Drug-Linker 13 and UC-961 antibody.

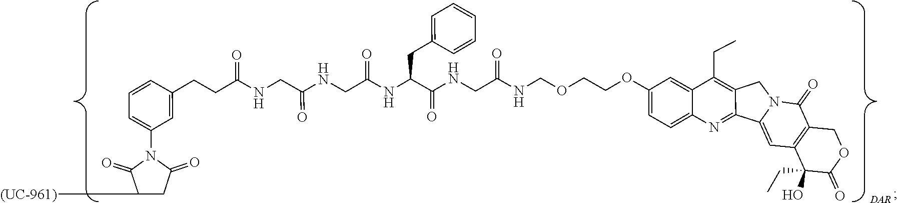

wherein the DAR value as determined by RP-HPLC was 7.7.

### Example 9: Preparation of Antibody-Drug Conjugate (ADC)-3 Using Drug-Linker 15 and UC-961

ADC-3 was prepared using example 6 and the Drug-Linker 15 and UC-961 antibody.

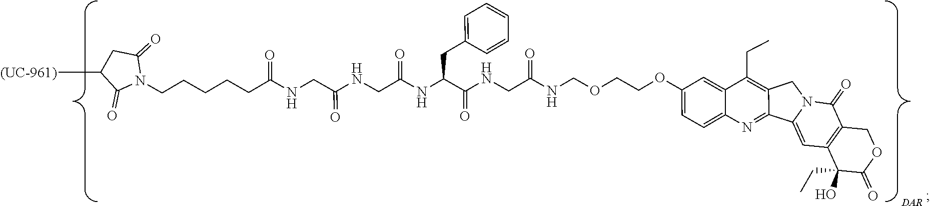

wherein the DAR value as determined by RP-HPLC was 7.8.

### Example 10: Preparation of Antibody-Drug Conjugate (ADC)-4 Using Drug-Linker 18 and UC-961

ADC-4 was prepared using example 6 and the Drug-Linker 18 and UC-961 antibody.

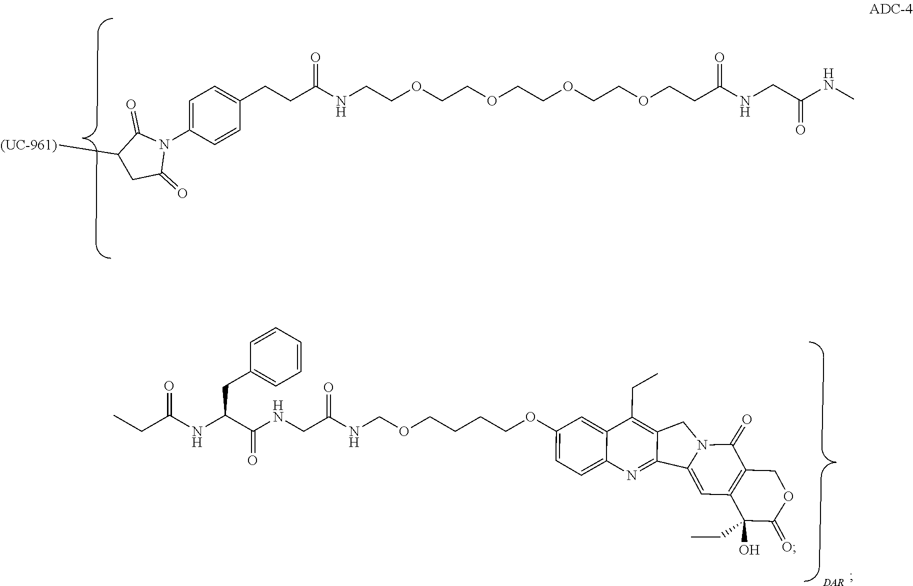

wherein the DAR value as determined by RP-HPLC was 7.8.

### Example 11: Preparation of Antibody-Drug Conjugate (ADC)-5 ADC-5 was Prepared Using Example 6 and UC-961 Antibody

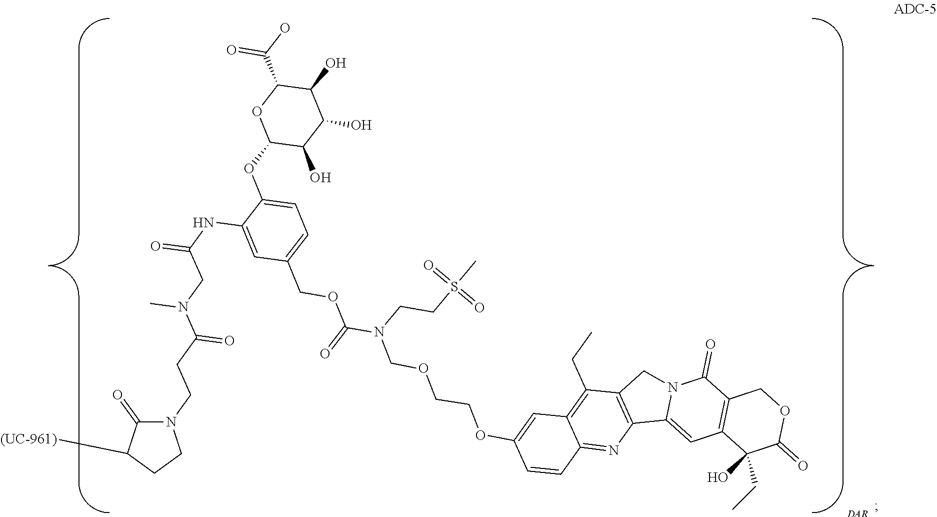

wherein the DAR value as determined by RP-HPLC was 7.8.

### Example 12: Preparation of Antibody-Drug Conjugate (ADC)-6 Using Drug-Linker 15 and PTK-7 Antibody

ADC-6 was prepared using example 6 and the Drug-Linker 15 and PTK7 antibody.

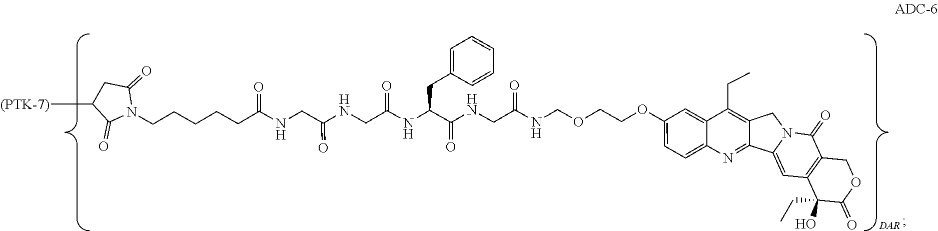

Wherein the DAR value as determined by RP-HPLC was 7.9.

### Characterization of Conjugates

### Example 13: Plasma Stability

The plasma stability tests illustrate the stability of the various ADCs in mouse and human plasma. ADCs were tested in both mouse and human plasma.

ADCs were incubated in IgG depleted plasma at a concentration of 50 g/mL in a volume of 300 μL. IgG depletion had been performed using a HiTrap Protein G Column (Cytiva). The samples were incubated at 37 °C in Eppendorf tubes for periods of 0, 1, 3, 7 and 15 days. After the appropriate time incubation, samples were transferred to a −80 °C freezer until they could be processed. Samples were incubated in each timepoint in duplicate.

To separate the ADCs from the plasma, 100 μL of each sample were mixed with 100 L Protein A magnetic bead slurry (Thermo Pierce) and 900 μL of Sodium Phosphate (50 mM, pH7) with shaking for two hours at room temperature. The ADC should then bind to the beads allowing the excess plasma to be discarded. The beads were then washed to remove nonspecific binding with 1 mL of 0.1% Triton, 0.1% IPA, and then twice in 1 mL PBS for 30 minutes each with shaking. The ADC was then eluted from the beads in 100 μL of a low pH, high organic mix (40 mM Glycine, 2% Formic Acid 50% Acetonitrile) for 60 minutes. The bead slurry was then centrifuged and 50 μL supernatant was injected onto the LC-MS.

The LC used a 50 mm×2.1 bioZen XB-C8 column over a seven-minute gradient of H2O and Acetonitrile each with 0.1% formic acid. The MS was operated in intact protein mode with a source temperature of 500 °C, delustering potential of 200 V, and a mass range of 900-4500 m/z.

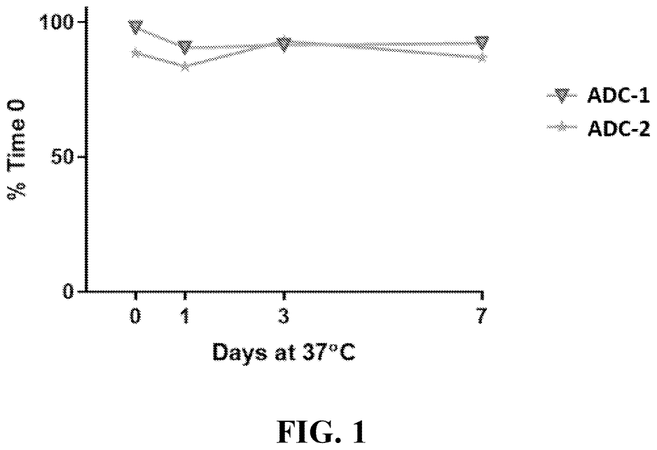

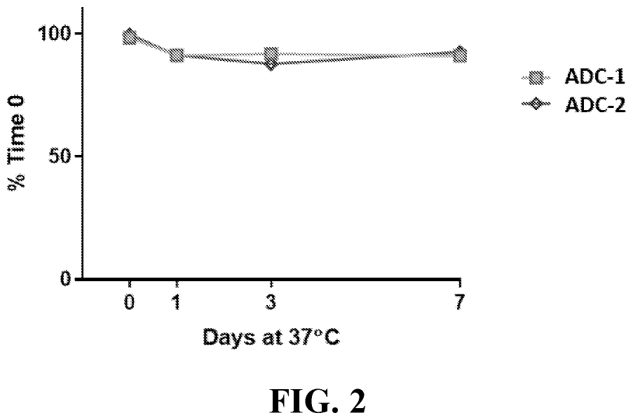

Processing was performed in Sciex OS and stability results were shown in FIG. 1, for mouse plasma and FIG. 2, for human plasma.

### Example 14: Cellular Binding

The ability of ADCs to bind to Jeko-1 cells was measured using in vitro cellular binding assay.

For each Jeko-1 cell line, 0.5 million cells are plated in 50 μL on each well of a 96-well deep-well plate (Thermo Scientific #249946). A 1:3 dilution series of primary antibody is made starting with 1-μg/mL starting stock. Thereafter, 50 μL of different concentrations of primary antibody (from 17 μg/mL to 1000 ng/mL final concentration) are placed over the 50 μL containing the cells. Cells and antibodies are mixed and incubated on ice for 20 minutes. For the first wash, 300 μL of FACS buffer is added and cells are centrifuged at 500×g for 5 minutes at 4 °C. The supernatant is discarded, and cells are resuspended in 400 μL of FACS buffer for an additional wash. After the second wash, cells are resuspended in 100 μL of goat anti-human IgG secondary phycoerythrin (PE) antibodies (ThermoFisher Scientific #12-4998-82) at 1 μg/mL final concentration and incubated on ice (in the dark) for 20 minutes. Cells are washed twice as described previously and analyzed on the BD FACSVerse with Flowjo software, Version 10. The percentage of maximal binding relative to the highest concentration was graphed and half-maximal effective concentrations (EC₅₀) values are determined using GraphPad Prism Version 7.

### Example 15: Cytotoxicity Measurement

The ability of compounds 4 and 6 to inhibit cell growth was measured using in vitro cytotoxicity assay.

Cells were cultured in log phase growth and split into 96-well plates. Each cell-line was plated at a slightly different concentration but ranged from 5×10³ to 50×10⁴ cells/well. Cells, in duplicate, were incubated with 3-fold serial dilution of a particular immunoconjugate starting at 3000 or 1000 nanomolar (3000, 1000, 333, 111, 37, 12.3, 4.1, 1.37, 0.46, 0.15 nanomolar) for 72 hours at 37 °C and 5% CO₂. After treatment the cells were incubated with an equal volume of CellTiter-Glo® reagent (Promega Inc.) for 15 minutes at room temperature, and viability was determined by a luminometer. EC50 values for SKBR3, a breast cancer cell line was shown in

### Table 1.

TABLE 1

| Compound # | EC50 in SKBR3 cells |
|---|---|
| 4 | 3.8 nM |
| 6 | 22 nM |

### Example 16: Internalization

MDA-MB-468 cells are harvested, washed with cold PBS and resuspended at a concentration of 1×10⁷ cells/mL in cold FACS buffer comprising PBS and 2% FBS. Aliquots of 1×10⁶ cells are added to microcentrifuge tubes or wells. Primary antibody is diluted to create 10× stock solutions of 300 μg/mL or 1 mg/mL to allow addition of 10 μl of each solution to the appropriate tubes.

Control groups comprised of unstained and secondary antibody only (goat anti-human IgG-PE, Fc-gamma specific) (ThermoFisher Scientific #12-4998-82). Test groups comprised cells are subjected to the following conditions with evaluation at 30 μg/mL (203 nM) or 100 μg/mL (676 nM) of primary antibody depending upon the experiment: Cells are kept on ice for 20 minutes in addition to controls centrifuged at 300×g for 4 minutes, washed twice with 200 μl FACS buffer, resuspended in 100 μl of FACS buffer, and incubated at 37 °C for 30, 60, 120, or 240 minutes. After incubation, cells are centrifuged at 250×g and washed twice with FACS buffer and resuspended in 100 μl of FACS buffer. A 10× stock of the secondary antibody is diluted 1:2000 in FACS buffer and 10 μl per tube was added to the appropriate tubes. Cells are incubated on ice for 20 minutes, washed twice with FACS buffer and resuspended in 100 μl of fixation buffer (4% paraformaldehyde in PBS). FACS analysis is then performed, assessing median fluorescence intensity (MFI). The relative magnitude of primary antibody internalization is determined by comparing MFI values for each timepoint with that for the primary antibody control at Time 0.

### Example 17: DAR by RP-HPLC

RP-HPLC of the ADCs was used to determine the drug to antibody ratio (DAR) of each ADC. As shown in the below Table 2, reversed phase HPLC was used to obtain the DAR and the monomer content of the ADCs.

RP-HPLC conditions: Column—Phenomenex Kinetex 100 Å, 50×4.6 mm, 2.6 μm, Part Number: PL1912-1502. MPA—0.1% TFA/H20, MPB—0.1% TFA/CAN

Method: Flow rate—1 mL/min, Gradient—see Table 3. Column temp. —50 C.

Sample temp—RT. DAD 214 nm, BW 16 nm; Reference 440 nm, BW 80 nm; Peak width >0.4 min (8 s response time (0.62 Hz); Spectrum: 200—600 nm, step 1.2 nm, slit 8 nm.

Sample—neat injection, ~5 ug.

TABLE 2
Monomer content and DAR of various ADCs

| ADC | Monomer (%) | DAR |
|---|---|---|
| ADC-1 | 96 | 7.8 |
| ADC-2 | 94 | 7.7 |
| ADC-3 | 97 | 7.8 |
| ADC-4 | 97 | 7.8 |
| ADC-5 | 97 | 7.8 |
| ADC-6 | 98 | 7.9 |

TABLE 3
RP-HPLC Method Flow rate Gradient

| Time (min) | B % |
|---|---|
| 0 | 30 |
| 2 | 30 |
| 10 | 41 |
| 11.5 | 90 |
| 15.5 | 90 |
| 16.5 | 30 |
| 20 | 30 |

### Example 18: In Vivo Efficacy

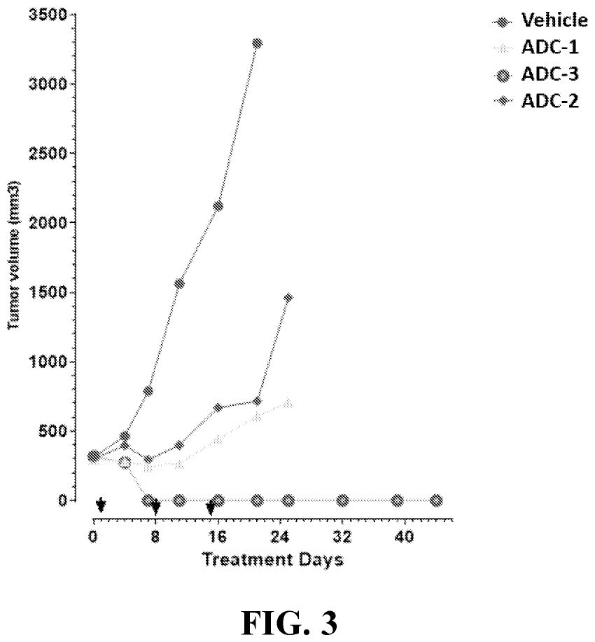

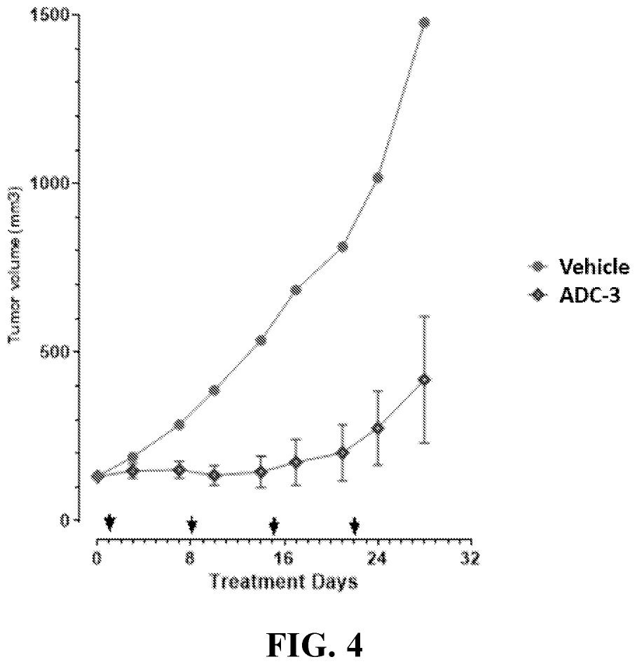

The antitumor activity of ADCs was evaluated in the xenograft models of Jeko-1 mantle cell lymphoma and SK-OV3 ovarian tumors. The tumor bearing mice were randomized based on their individual tumor and given iv injection dose. As shown in FIG. 3, at 10 mg/Kg, once weekly iv injection dose for 3 weeks (days 1, 8, 15), ADCs were shown to have antitumor activity in Jeko-1 model. Whereas in FIG. 4, at 20 mg/Kg, once weekly iv injection dose for 4 weeks (days 1, 8, 15, 22), ADC-3 was shown to have antitumor activity in SK-OV3 model.

What is claimed is:

1. A compound of Formula (I): 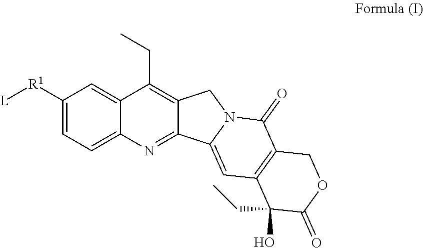
or a pharmaceutically acceptable salt thereof, wherein;
R¹ is selected from —O—CH₂—CH₂—O— and —NH—C(O)—CH₂—O—;
L is L²-(L^{2B})_{z}-L³-L⁴-(L⁵)ₘ-(L⁶)ₙ-(L⁷)ₚ-R²;
L² is selected from a C₁₋₆ alkylene;
L^{2B} is selected from NR⁴C(O)O—C₁₋₆ alkylene-phenylene, wherein the phenylene is optionally substituted with one or more R⁵;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocyclene;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
R⁴ is selected from hydrogen and C₁₋₆ alkyl optionally substituted with one or more SO₂C₁₋₆ alkyl;
each R⁵ is independently selected from a sugar;
m is selected from 0 and 1;
n is selected from 0 and 1;
p is selected from 0 and 1;
q is selected from 0 to 8;
s is selected from 0 and 1; and
z is selected from 0 and 1.

2. The compound or salt of claim 1, wherein Formula (I) is represented by 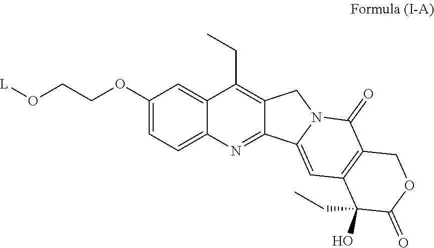
or a pharmaceutically acceptable salt thereof.

3. The compound or salt of claim 1, wherein Formula (I) is represented by 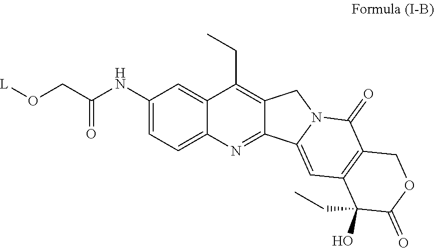
or a pharmaceutically acceptable salt thereof.

4. The compound or salt of any one of claims 1 to 3, wherein L is L²-L³-L⁴-(L⁵)ₘ-(L⁶)ₙ-(L⁷)ₚ-R²;

5. The compound or salt of any one of claims 1 to 4, wherein L is L²-L³-L⁴-R².

6. The compound or salt of any one of claims 1 to 4, wherein L is L²-L³-L⁴-L⁷-R².

7. The compound or salt of any one of claims 1 to 4, wherein L is L²-L³-L⁴-L⁵-L⁶-L⁷-R².

8. The compound or salt of any one of claims 1 to 4, wherein L is L²-L^{2B}-L³-L⁴-R²;

9. The compound or salt of any one of claims 1 to 8, wherein L² is C₁ alkylene.

10. The compound or salt of any one of claims 1 to 9, wherein L³ is selected from a residue comprising 1 to 5 amino acids.

11. The compound or salt of any one of claims 1 to 10, wherein the amino acid is a natural amino acid.

12. The compound or salt of any one of claims 1 to 11, wherein the amino acid is selected from an alpha amino acid and beta amino acid.

13. The compound or salt of claim 12, wherein the amino acid is selected from a group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, citrulline, sarcosine, and β-Alanine.

14. The compound or salt of any one of claims 1 to 13, wherein the amino acids of L³ are selected from a group consisting of glycine and phenylalanine.

15. The compound or salt of any one of claims 1 to 14, wherein the residue of L³ comprises 4 amino acids.

16. The compound or salt of any one of claims 1 to 15, wherein the residue of L³ comprises at least two different amino acids.

17. The compound or salt of any one of claims 1 to 16, wherein L³ is

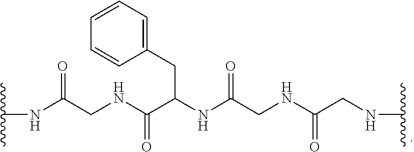 [SMILES: *NCC(=O)NCC(=O)NC(Cc1ccccc1)C(=O)NCC(=O)N*]

18. The compound or salt of claim 13, wherein the amino acids of L³ are selected from a group consisting of sarcosine.

19. The compound or salt of claim 18, wherein the residue of L³ comprises 1 amino acid.

20. The compound or salt of claim 19, wherein L³ is

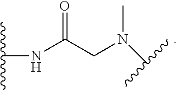 [SMILES: *NC(=O)CN(*)C]

21. The compound or salt of claim 1 or 8, wherein L^{2B} is selected from NR⁴C(O)O—C₁ alkylene-phenyl, wherein the phenyl is substituted with one R⁵.

22. The compound or salt of claim 21, wherein R⁵ is

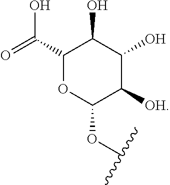

23. The compound or salt of any one of claims 1 to 22, wherein L⁴ is selected from

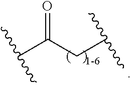

24. The compound or salt of any one of claims 1 to 23, wherein L⁴ is selected from —C(O)—(CH₂)₂— and —C(O)—(CH₂)₅—.

25. The compound or salt of claims 1, 4, or 7, wherein L⁵ is —(O—CH₂—CH₂—)₄—NH—.

26. The compound or salt of claims 1, 4, 7, or 25, wherein L⁶ is selected from

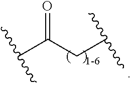

27. The compound or salt of claims 1, 4, 7, or 26, wherein L⁶ is selected from —C(O)—(CH₂)₂—and —C(O)—(CH₂)₅—.

28. The compound or salt of claim 27, wherein L⁶ is selected —C(O)—(CH₂)₂—.

29. The compound or salt of any one of claims 1, 4, 6, or 7, wherein L⁷ is phenylene.

30. The compound or salt of any one of claims 1 to 29, wherein R² is selected from an optionally substituted 5-membered heterocycle.

31. The compound or salt of claim 30, wherein R² is

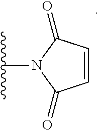 [SMILES: *N1C(=O)C=CC1=O]

32. The compound or salt of claim 1, wherein the compound is selected from 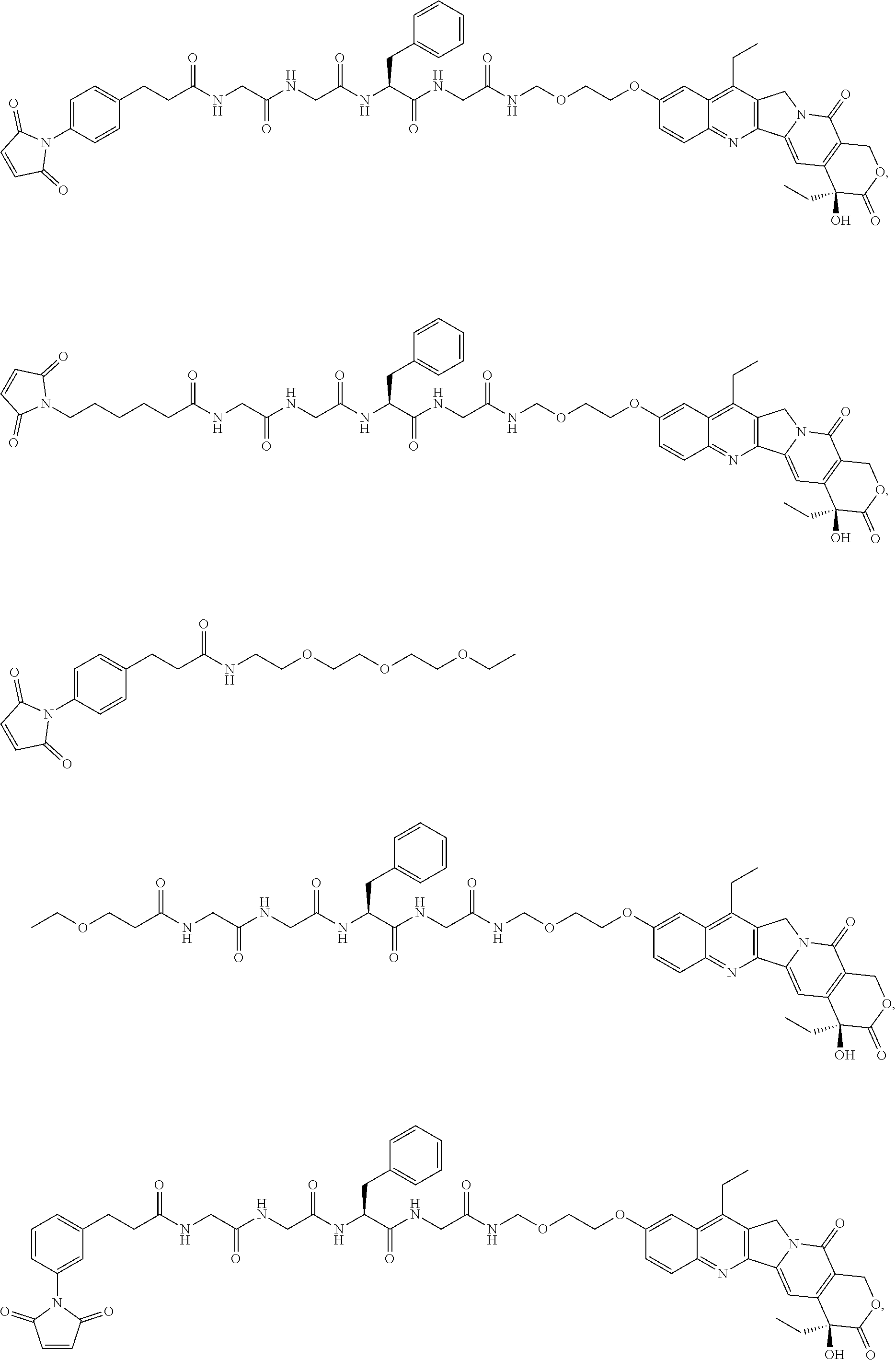 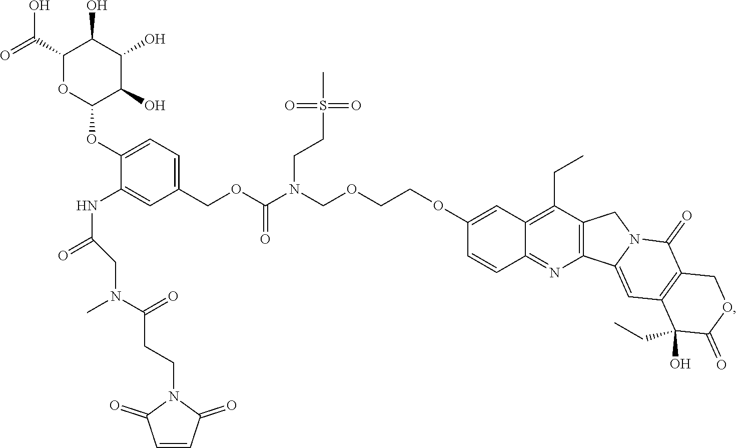 [SMILES: CCc1c2c(nc3ccc(OCCOCN(CCS(C)(=O)=O)C(=O)OCc4ccc(O[C@@H]5O[C@H](C(=O)O)[C@@H](O)[C@H](O)[C@H]5O)c(NC(=O)CN(C)C(=O)CCN5C(=O)C=CC5=O)c4)cc13)-c1cc3c(c(=O)n1C2)COC(=O)[C@]3(O)CC]
or a pharmaceutically acceptable salt of any one thereof.

33. The compound or salt of claim 1, wherein the compound is selected from 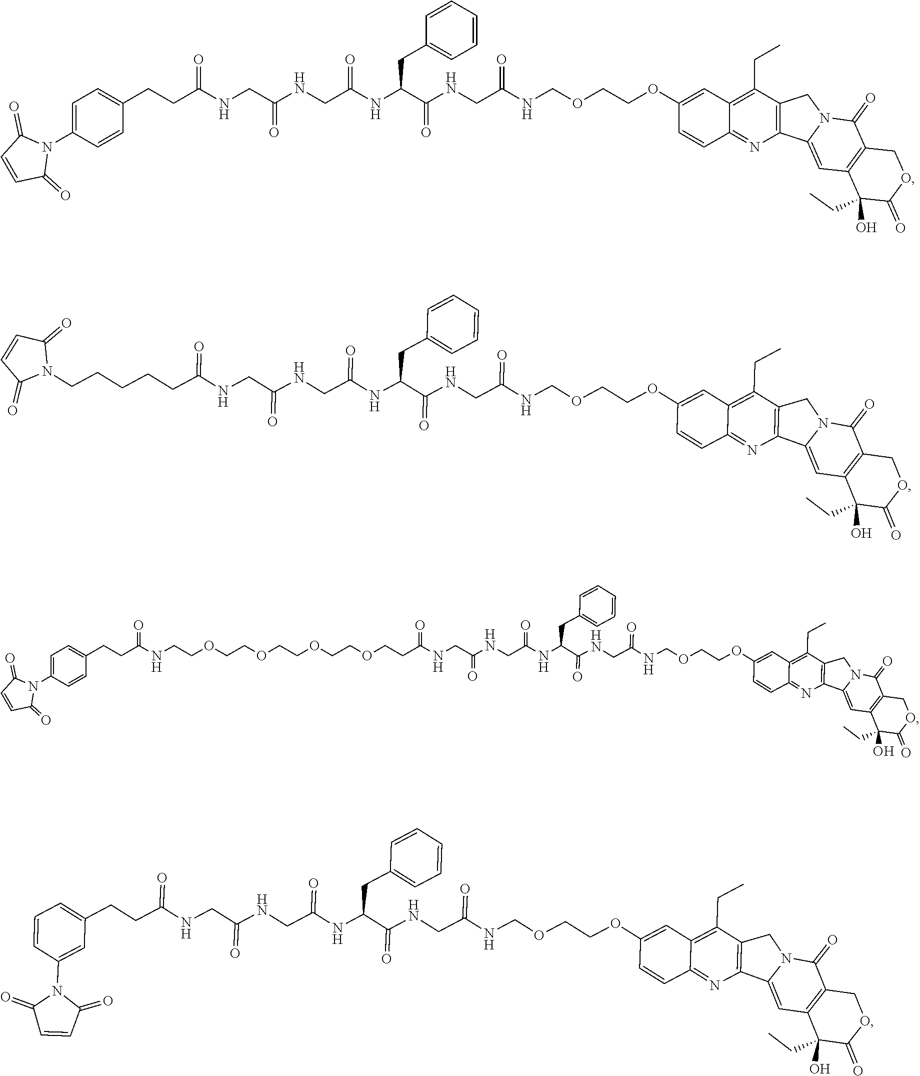
or a pharmaceutically acceptable salt of any one thereof.

34. The compound or salt of claim 1, wherein the compound is selected from 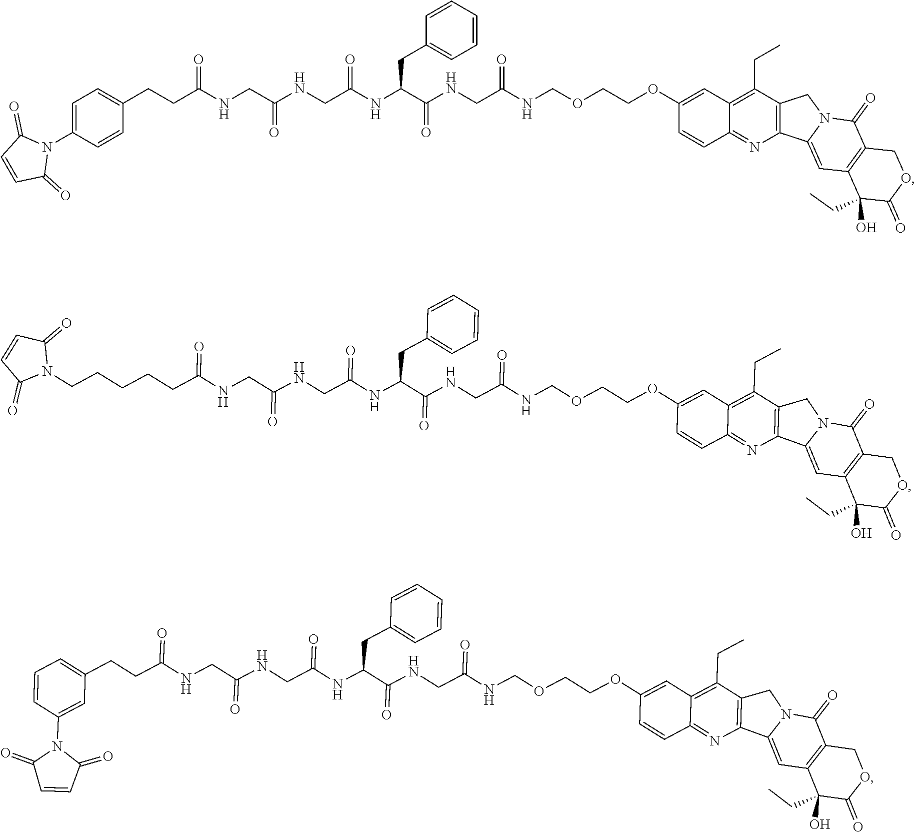 [SMILES: CCc1c2c(nc3ccc(OCCOCNC(=O)CNC(=O)[C@H](Cc4ccccc4)NC(=O)CNC(=O)CNC(=O)CCc4ccc(N5C(=O)C=CC5=O)cc4)cc13)-c1cc3c(c(=O)n1C2)COC(=O)[C@]3(O)CC ; CCc1c2c(nc3ccc(OCCOCNC(=O)CNC(=O)[C@H](Cc4ccccc4)NC(=O)CNC(=O)CNC(=O)CCCCCN4C(=O)C=CC4=O)cc13)-c1cc3c(c(=O)n1C2)COC(=O)[C@]3(O)CC ; CCc1c2c(nc3ccc(OCCOCNC(=O)CNC(=O)[C@H](Cc4ccccc4)NC(=O)CNC(=O)CNC(=O)CCc4cccc(N5C(=O)C=CC5=O)c4)cc13)-c1cc3c(c(=O)n1C2)COC(=O)[C@]3(O)CC]
or a pharmaceutically acceptable salt of any one thereof.

35. The compound or salt of claim 1, wherein
R¹ is selected from —O—CH₂—CH₂—O—;
L is L²-L³-L⁴-(L⁵)ₘ-(L⁶)ₙ-(L⁷)ₚ-R²;
L² is selected from a C₁₋₆ alkylene;
L³ is selected from a residue comprising 3 to 5 amino acids;
L⁴ is selected from 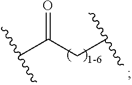
L⁵ is selected from an (O—CH₂—CH₂—)_{q}—(NR³)ₛ;
L⁶ is selected from 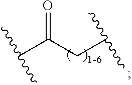
L⁷ is phenylene;
R² is selected from maleimide;
R³ is selected from hydrogen and C₁₋₆ alkyl;
m is selected from 0 and 1;
n is selected from 0 and 1;
p is selected from 0 and 1;
q is selected from 2 to 6; and
s is selected from 0 and 1.

36. The compound or salt of claim 1 or 35, wherein
R¹ is selected from —O—CH₂—CH₂—O—;
L is L²-L³-L⁴-(L⁵)ₘ-(L⁶)ₙ-(L⁷)ₚR²;
L² is 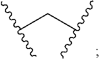 [SMILES: *C*]
L³ is 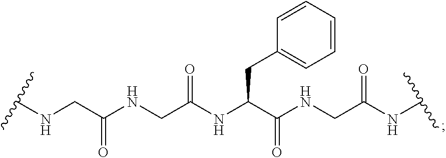
L⁴ is selected from 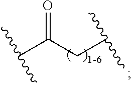
L⁵ is 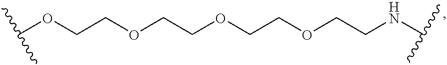
m is selected from 0 and 1;
L⁶ is 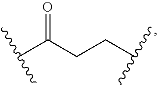
n is selected form 0 and 1;
L⁷ is 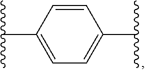
p is selected from 0 and 1;
R² is

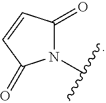

37. The compound or salt of claim 1, or 35 to 36, wherein
R¹ is selected from —O—CH₂—CH₂—O—;
L is L²-L³-L⁴-(L⁵)ₘ-L⁶)ₙ-(L⁷)ₚR²;
L² is 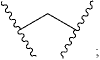 [SMILES: *C*]
L³ is 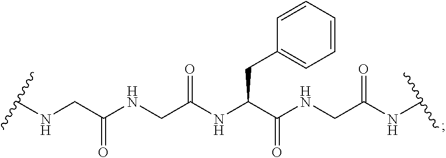
L⁴ is selected from 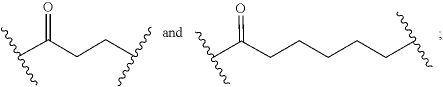
L⁵ is 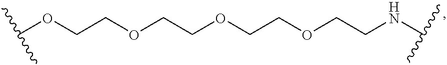
m is selected from 0 and 1;
L⁶ is 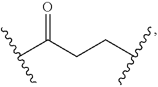
n is selected form 0 and 1;
L⁷ is 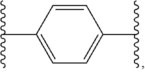
p is selected from 0 and 1;
R² is

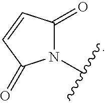

38. The compound or salt of any one of claims 1 to 37, further comprising a ligand (Lg).

39. The compound or salt of any one of claims 1 to 37, wherein the compound or salt is further modified by a ligand.

40. The compound or salt of any one of claims 38 to 39, wherein the compound or salt is covalently attached to a ligand.

41. The compound or salt of any one of claims 38 to 40, wherein the compound or salt reacts with the ligand to form a covalent bond.

42. The compound or salt of any one of claims 38 to 41, wherein the ligand is selected from an antibody or an antigen-binding fragment thereof.

43. The compound or salt of any one of claims 38 to 42, wherein Formula (I) is represented by 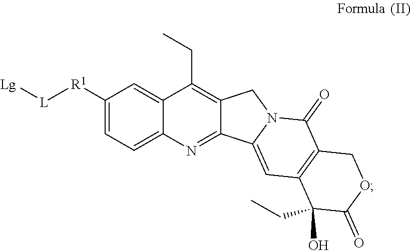
or a pharmaceutically acceptable salt thereof, wherein;
Lg is the ligand.

44. A compound of Formula (III): 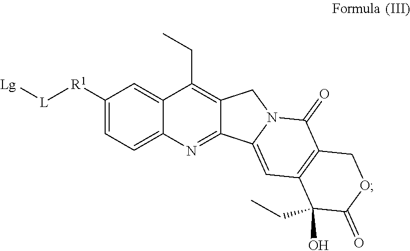
or a pharmaceutically acceptable salt thereof, wherein;
Lg is a ligand;
R¹ is selected from —O—CH₂—CH₂—O— and —NH—C(O)—CH₂—O—;
L is L²-(L^{2B})_{z}-L³-L⁴-(L⁵)ₘ-(L⁶)ₙ-(L⁷)ₚ-R²;
L² is selected from a C₁₋₆ alkylene;
L^{2B} is selected from NR⁴C(O)O—C₁₋₆ alkylene-phenylene, wherein the phenylene is optionally substituted with one or more R⁵;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an —(O—CH₂—CH₂—)_{q}—(NR³)ₛ—;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocycle;
R² is selected from an optionally substituted 5- to 6-membered heterocyclene, wherein the 5- to 6-membered heterocyclene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
R⁴ is selected from C₁₋₆ alkyl optionally substituted with one or more SO₂C₁₋₆ alkyl;
each R⁵ is independently selected from a sugar;
m is selected from 0 and 1;
n is selected from 0 and 1;
p is selected from 0 and 1;
q is selected from 0 to 8;
s is selected from 0 and 1; and
z is selected from 0 and 1.

45. The compound or salt of claim 44, wherein R¹ is —O—CH₂—CH₂—O—.

46. The compound or salt of claim 44, wherein R¹ is —NH—C(O)—CH₂—O—.

47. The compound or salt of any one of claims 44 to 46, wherein Formula (III) is represented by 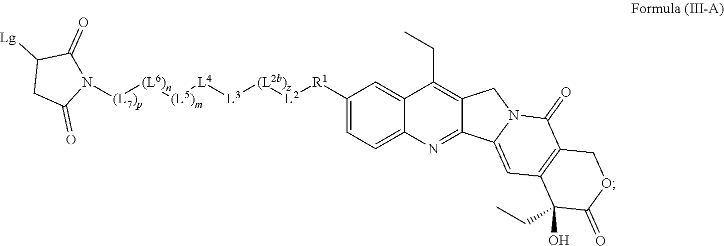
or a pharmaceutically acceptable salt thereof.

48. The compound or salt of any one of claims 44 to 46, wherein L is L²-L³-L⁴-(L⁵)ₘ-(L⁶)ₙ-(L⁷)ₚ-R².

49. The compound or salt of any one of claims 44 to 46, wherein L is L²-L³-L⁴-R².

50. The compound or salt of any one of claims 44 to 46, wherein L is L²-L³-L⁴-L⁷-R².

51. The compound or salt of any one of claims 44 to 46, wherein L is L²-L³-L⁴-L⁵-L⁶-L⁷-R².

52. The compound or salt of any one of claims 44 to 46, wherein L is L²-L^{2B}-L³-L⁴-R².

53. The compound or salt of any one of claims 44 to 52, wherein L² is C₁ alkylene.

54. The compound or salt of any one of claims 44 to 53, wherein L³ is selected from a residue comprising 1 to 5 amino acids.

55. The compound or salt of any one of claims 44 to 54, wherein L³ is selected from a residue comprising 3 to 5 amino acids.

56. The compound or salt of any one of claims 44 to 55, wherein the amino acid is a natural amino acid.

57. The compound or salt of any one of claims 44 to 56, wherein the amino acid is selected from an alpha amino acid and beta amino acid.

58. The compound or salt of any one of claims 44 to 57, wherein the amino acid is selected from a group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, citrulline, sarcosine, and β-Alanine.

59. The compound or salt of any one of claims 44 to 58, wherein the amino acids of L³ are selected from a group consisting of glycine and phenylalanine.

60. The compound or salt of any one of claims 44 to 59, wherein the residue of L³ has 4 amino acids.

61. The compound or salt of any one of claims 44 to 60, wherein the residue of L³ includes at least two different amino acids.

62. The compound or salt of any one of claims 44 to 61, wherein L³ is

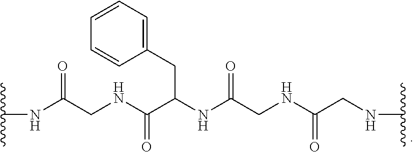 [SMILES: *NCC(=O)NCC(=O)NC(Cc1ccccc1)C(=O)NCC(=O)N*]

63. The compound or salt of claim 58, wherein the amino acids of L³ are selected from a group consisting of sarcosine.

64. The compound or salt of claim 63, wherein the residue of L³ comprises 1 amino acid.

65. The compound or salt of claim 64, wherein L³ is

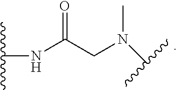 [SMILES: *NC(=O)CN(*)C]

66. The compound or salt of claim 1 or 52, wherein L^{2B} is selected from NR⁴C(O)O—C₁ alkylene-phenylene, wherein the phenylene is substituted with one R⁵.

67. The compound or salt of claim 66, wherein R⁵ is

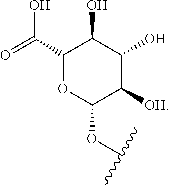

68. The compound or salt of any one of claims 44 to 67, wherein L⁴ is selected from

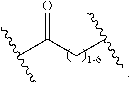

69. The compound or salt of any one of claims 44 to 68, wherein L⁴ is selected from —C(O)—(CH₂)₂— and —C(O)—(CH₂)₅—.

70. The compound or salt of claim 44 or 51, wherein L⁵ is —(O—CH₂—CH₂—)₄—NH—.

71. The compound or salt of claim 44, 51 or 70, wherein L⁶ is selected from

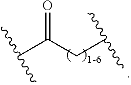

72. The compound or salt of claim 44 or 71, wherein L⁶ is selected from —C(O)—(CH₂)₂— and —C(O)—(CH₂)₅—.

73. The compound or salt of claim 44, 51, or 70-72, wherein L⁶ is selected —C(O)—(CH₂)₂—.

74. The compound or salt of any one of claims 1, 50, or 51, wherein L⁷ is phenylene.

75. The compound or salt of any one of claims 1 to 2, wherein
R¹ is selected from —O—CH₂—CH₂—O—;
L is L²-L³-L⁴-(L⁵)ₘ-(L⁶)ₙ-(L⁷)ₚ-R²;
L² is selected from a C₁₋₆ alkylene;
L³ is selected from a residue comprising 3 to 5 amino acids;
L⁴ is selected from 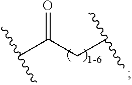
L⁵ is selected from an (O—CH₂—CH₂—)_{q}—(NR³)ₛ;
L⁶ is selected from 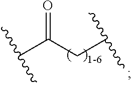
L⁷ is phenylene;
R² is selected from maleimide;
R³ is selected from hydrogen and C₁₋₆ alkyl;
m is selected from 0 and 1;
n is selected from 0 and 1;
p is selected from 0 and 1;
q is selected from 2 to 6; and
s is selected from 0 and 1.

76. The compound or salt of any one of claims 1 to 2, or 75, wherein
R¹ is selected from —O—CH₂—CH₂—O—;
L is L²-L³-L⁴-(L⁵)ₘ-(L⁶)ₙ-(L⁷)ₚ-R²;
L² is 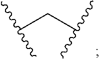 [SMILES: *C*]
L³ is 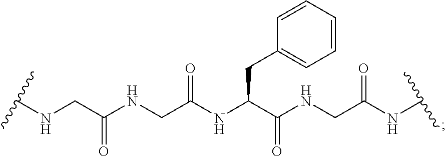
L⁴ is selected from 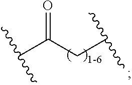
L⁵ is 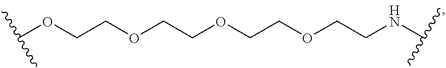
m is selected from 0 and 1;
L⁶ 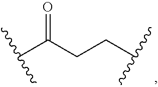
n is selected form 0 and 1;
L⁷ is selected from 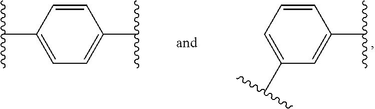 [SMILES: *c1ccc(*)cc1 ; *c1cccc(*)c1]
p is selected from 0 and 1;
R² is

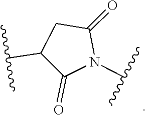 [SMILES: *C1CC(=O)N(*)C1=O]

77. The compound or salt of any one of claims 1 to 2, or 75 to 76, wherein
R¹ is selected from —O—CH₂—CH₂—O—;
L is L²-L³-L⁴-(L⁵)ₘ-(L⁶)ₙ-(L⁷)ₚ-R²;
L² is 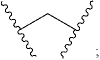 [SMILES: *C*]
L³ is 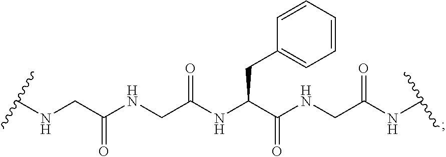
L⁴ is selected from 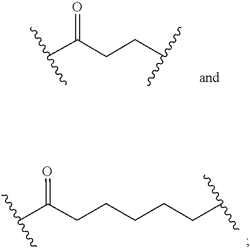
L⁵ is 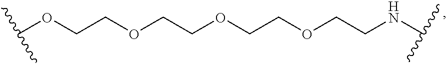
m is selected from 0 and 1;
L⁶ 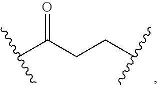
n is selected form 0 and 1;
L⁷ 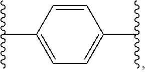
p is selected from 0 and 1;
R²

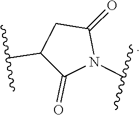 [SMILES: *C1CC(=O)N(*)C1=O]

78. The compound or salt of claim 44, wherein the compound is represented by Formula (V-A) 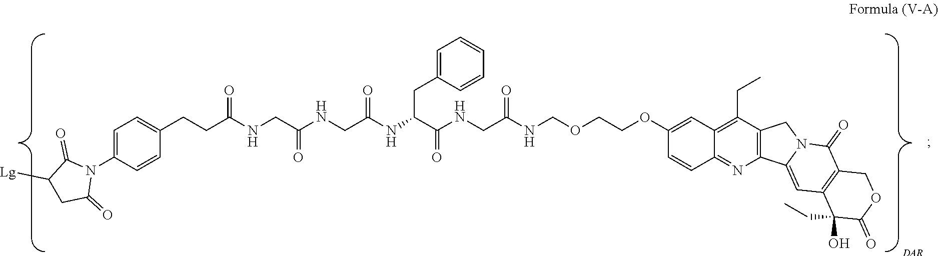
or a pharmaceutically acceptable salt thereof, wherein the DAR is selected from about 1-8.

79. The compound or salt of claim 44, wherein the compound is represented by Formula (V-B) 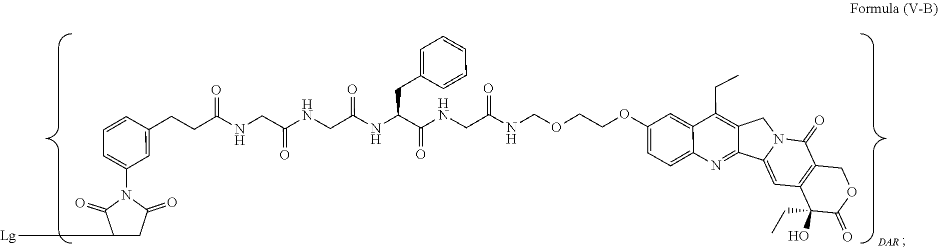
or a pharmaceutically acceptable salt thereof, wherein the DAR is selected from about 1-8.

80. The compound or salt of claim 44, wherein the compound is represented by Formula (V-C): 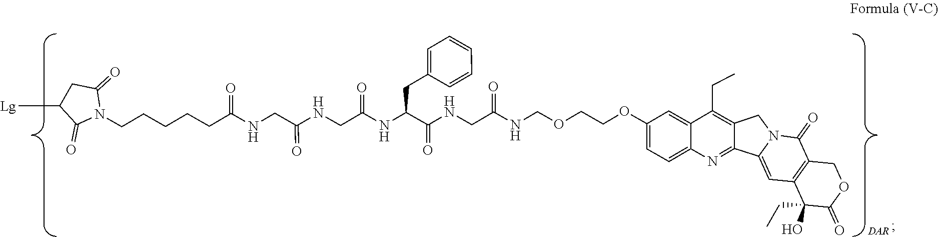
or a pharmaceutically acceptable salt thereof, wherein the DAR is selected from about 1-8.

81. The compound or salt of claim 44, wherein the compound is represented by Formula (V-D) 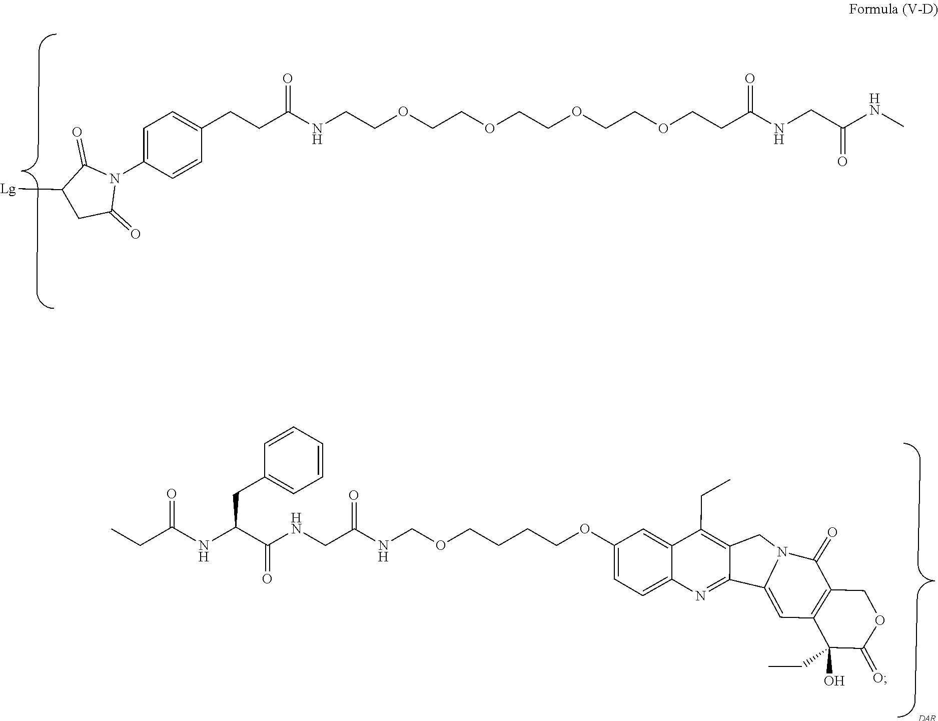
or a pharmaceutically acceptable salt thereof, wherein the DAR is selected from about 1-8.

82. The compound or salt of claim 44 or 78, wherein the compound is selected from: 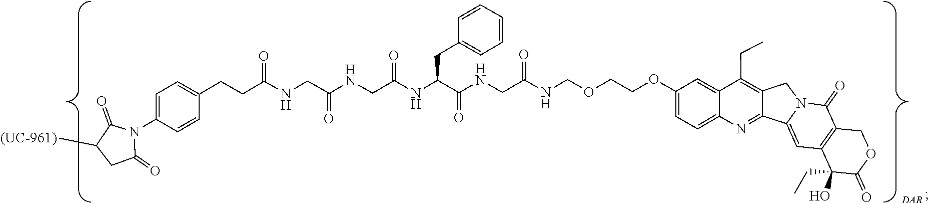
or a pharmaceutically acceptable salt thereof, wherein the DAR is selected from about 1-8.

83. The compound or salt of claim 44 or 79, wherein the compound is selected from: 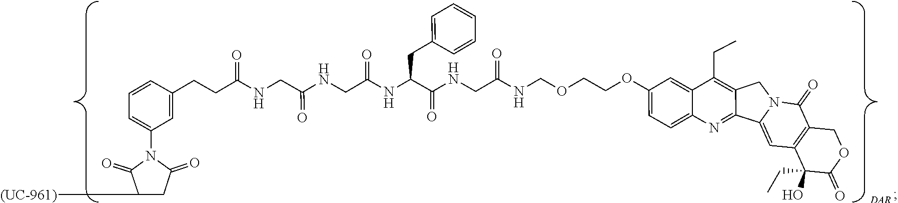
or a pharmaceutically acceptable salt thereof, wherein the DAR is selected from about 1-8.

84. The compound or salt of claim 44 or 80, wherein the compound is selected from: 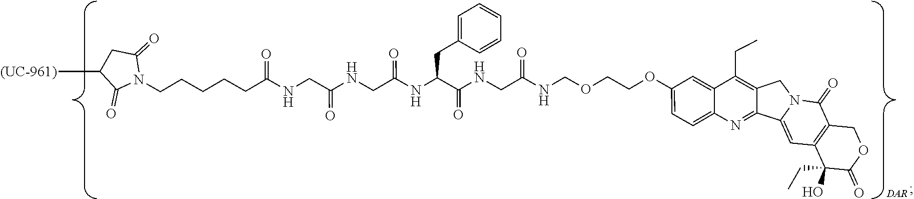
or a pharmaceutically acceptable salt thereof, wherein the DAR is selected from about 1-8.

85. The compound or salt of any one of claims 44 to 81, wherein the ligand is selected from an antibody or an antigen-binding fragment thereof.

86. The compound or salt of claim 85, wherein the ligand is selected from a group consisting of chimeric antibodies, humanized antibodies, and fully human antibodies.

87. The compound or salt of claim 85, wherein the antibody or antigen-binding fragment thereof binds to an antigen selected from the group comprising or consisting of CD19, CD20, CD22, CD30, CD37, CD79b, HER2, and PSMA.

88. The compound or salt of claim 85, wherein the antibody or antigen-binding fragment thereof binds to HER2.

89. The compound or salt of claim 85, wherein the antibody or antigen-binding fragment thereof is selected from UC-961, PTK-7, trastuzumab, brentuximab, loncastuximab, rosopatamab, rituximab, pinatuzumab, polatuzumab, and naratuximab.

90. The compound or salt of claim 85, wherein the antibody is trastuzumab.

91. A compound of Formula (IV): 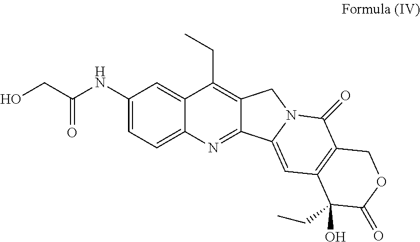 [SMILES: CCc1c2c(nc3ccc(NC(=O)CO)cc13)-c1cc3c(c(=O)n1C2)COC(=O)[C@]3(O)CC]
or a pharmaceutically acceptable salt thereof.

92. A pharmaceutical composition comprising a compound or salt of any one of claims 1 to 91 and a pharmaceutically acceptable excipient.

93. The use of a compound or salt of any one of claims 1 to 91, or the pharmaceutical composition of claim 92, in the treatment of a disease or disorder.

94. The use of a compound or salt of any one of claims 1 to 91, or the pharmaceutical composition of claim 92, in the treatment of a tumor.

95. The use of a compound or salt of any one of claims 1 to 91, or the pharmaceutical composition of claim 92, in the treatment of a cancer.

96. A method of treating a subject with a disease or disorder, comprising administering to the subject in need thereof a compound or salt of any one of claims 1 to 91 or a pharmaceutical composition of claim 92.

97. A method of treating a subject with a tumor, comprising administering to the subject in need thereof a compound or salt of any one of claims 1 to 91 or a pharmaceutical composition of claim 92.

98. A method of treating a subject with a cancer, comprising administering to the subject in need thereof a compound or salt of any one of claims 1 to 91 or a pharmaceutical composition of claim 92.

99. The method of claim 95 or 98, wherein the cancer is selected from the group consisting of lung cancer, kidney cancer, urethral cancer, colorectal cancer, prostate cancer, glioblastoma multiforme, ovarian cancer, pancreatic, breast, melanoma, liver, bladder, stomach, and esophageal cancers.

100. A compound of Formula (I)*: 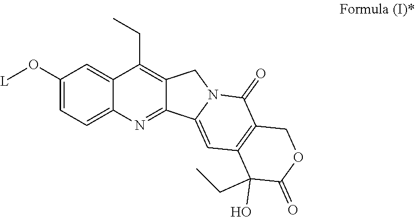
or a pharmaceutically acceptable salt thereof, wherein;
L is L²-L³-L⁴-(L⁵)ₘ-(L⁶)ₙ-(L⁷)ₚ-R²;
L² is selected from a C₁₋₆ alkylene;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocyclene;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
m is selected from 0 and 1;
n is selected from 0 and 1;
p is selected from 0 and 1;
q is selected from 0 to 8; and
s is selected from 0 and 1.

101. The compound or salt of claim 100, wherein Formula (I)* is represented by 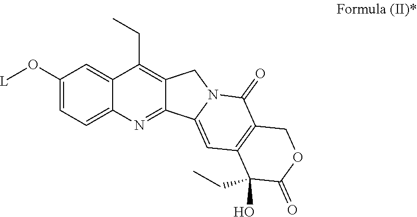
or a pharmaceutically acceptable salt thereof.

102. The compound or salt of any one of claims 100 to 101, wherein L is L²-L³-L⁴-R².

103. The compound or salt of any one of claims 100 to 101, wherein L is L²-L³-L⁴-L⁷-R².

104. The compound or salt of any one of claims 100 to 101, wherein L is L²-L³-L⁴-L⁵-L⁶-L⁷-R².

105. The compound or salt of any one of claims 100 to 104, wherein L² is C₁ alkylene.

106. The compound or salt of any one of claims 100 to 105, wherein L³ is selected from a residue comprising 1 to 5 amino acids.

107. The compound or salt of any one of claims 100 to 106, wherein the amino acid is a natural amino acid.

108. The compound or salt of any one of claims 100 to 107, wherein the amino acid is selected from an alpha amino acid and beta amino acid.

109. The compound or salt of claim 108, wherein the amino acid is selected from a group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, citrulline, sarcosine, and β-Alanine.

110. The compound or salt of any one of claims 100 to 109, wherein the amino acids of L³ are selected from a group consisting of glycine and phenylalanine.

111. The compound or salt of any one of claims 100 to 110, wherein the residue of L³ includes 4 amino acids.

112. The compound or salt of any one of claims 100 to 111, wherein the residue of L³ comprises at least two different amino acids.

113. The compound or salt of any one of claims 100 to 112, wherein L³ is

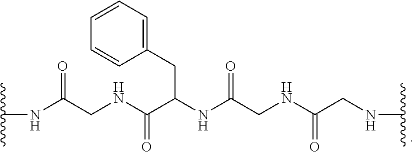 [SMILES: *NCC(=O)NCC(=O)NC(Cc1ccccc1)C(=O)NCC(=O)N*]

114. The compound or salt of any one of claims 100 to 113, wherein L⁴ is selected from —C(O)—(CH₂)₂— and —C(O)—(CH₂)₅—.

115. The compound or salt of claim 100 or 104, wherein L⁵ is —(O—CH₂—CH₂—)₄—NH—.

116. The compound or salt of claim 100 or 104, wherein L⁶ is selected from —C(O)—(CH₂)₂— and —C(O)—(CH₂)₅—.

117. The compound or salt of claim 116, wherein L⁶ is selected —C(O)—(CH₂)₂—.

118. The compound or salt of any one of claim 100 or 117, wherein L⁷ is phenylene.

119. The compound or salt of any one of claims 100 to 118, wherein R² is selected from an optionally substituted 5-membered heterocycle.

120. The compound or salt of claim 119, wherein R² is

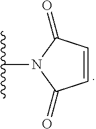 [SMILES: *N1C(=O)C=CC1=O]

121. The compound or salt of claims 100 to 120, wherein L is selected from 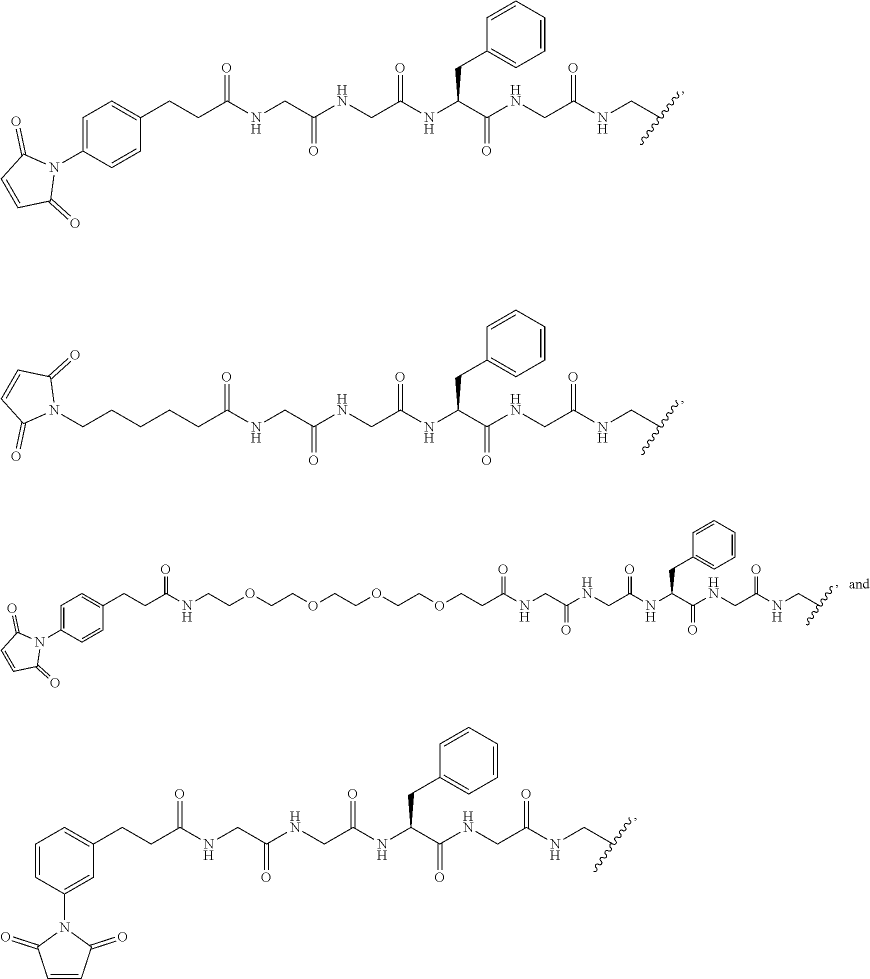 [SMILES: *CNC(=O)CNC(=O)[C@H](Cc1ccccc1)NC(=O)CNC(=O)CNC(=O)CCc1ccc(N2C(=O)C=CC2=O)cc1 ; *CNC(=O)CNC(=O)[C@H](Cc1ccccc1)NC(=O)CNC(=O)CNC(=O)CCCCCN1C(=O)C=CC1=O ; *CNC(=O)CNC(=O)[C@H](Cc1ccccc1)NC(=O)CNC(=O)CNC(=O)CCOCCOCCOCCOCCNC(=O)CCc1ccc(N2C(=O)C=CC2=O)cc1 ; *CNC(=O)CNC(=O)[C@H](Cc1ccccc1)NC(=O)CNC(=O)CNC(=O)CCc1cccc(N2C(=O)C=CC2=O)c1]
or a pharmaceutically acceptable salt of any one thereof.

122. The compound or salt of claim 100 or 121, wherein the compound is selected from 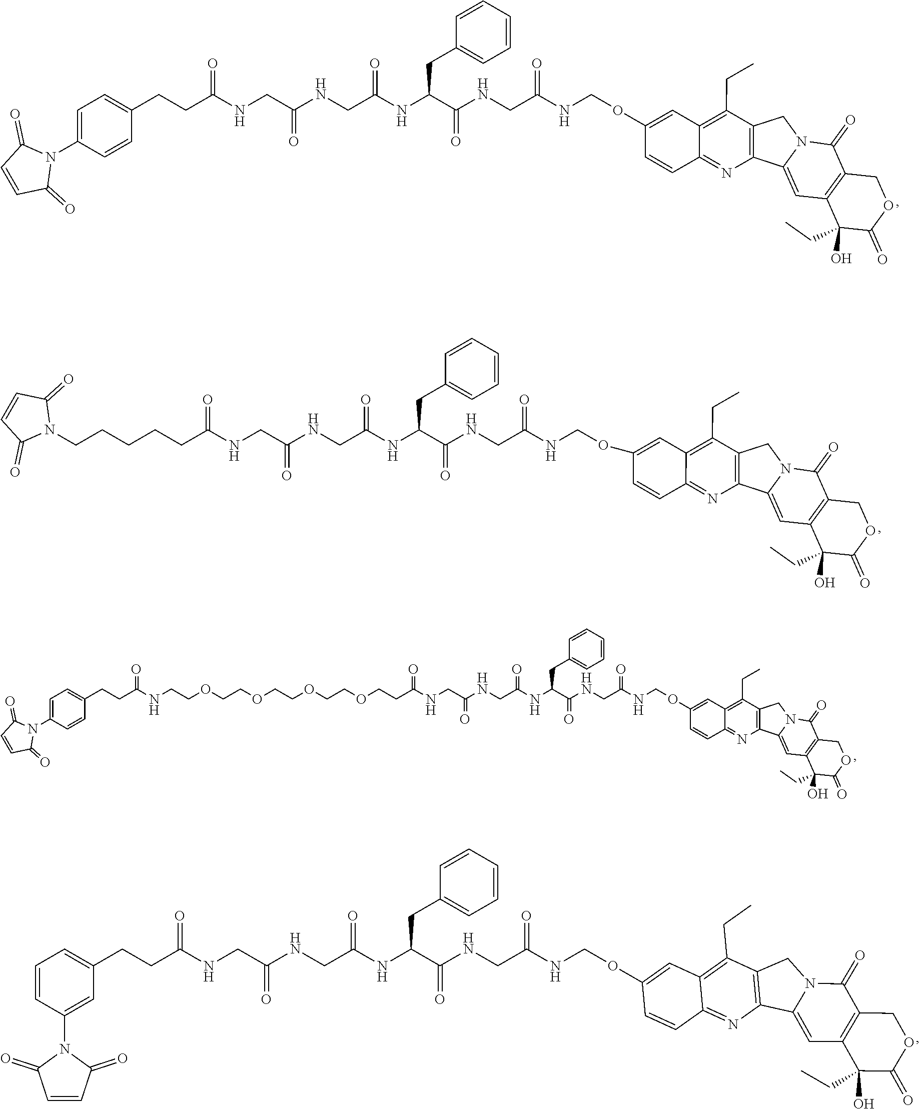
or a pharmaceutically acceptable salt of any one thereof.

123. The compound or salt of any one of claims 100 to 122, further comprising a ligand.

124. The compound or salt of any one of claims 100 to 123, wherein the compound or salt is further modified by a ligand.

125. The compound or salt of any one of claims 100 to 124, wherein the compound or salt is covalently attached to a ligand.

126. The compound or salt of any one of claims 100 to 125, wherein the compound or salt reacts with a ligand to form a covalent bond.

127. The compound or salt of any one of claims 100 to 126, wherein Formula (I)* is represented by 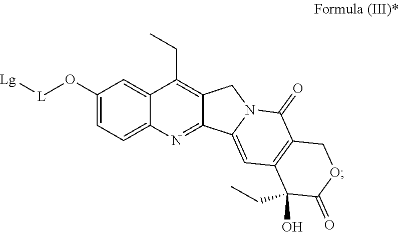
or a pharmaceutically acceptable salt thereof, wherein;
Lg is the ligand.

128. A compound of Formula (IV)*: 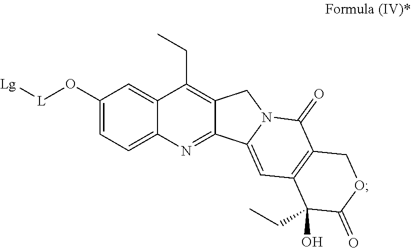
or a pharmaceutically acceptable salt thereof, wherein;
Lg is a ligand;
L is L²-L³-L⁴-(L⁵)ₘ-(L⁶)ₙ-(L⁷)ₚ-R²;
L² is selected from a C₁₋₆ alkylene;
L³ is selected from a residue comprising 1 to 7 amino acids;
L⁴ is selected from an optionally substituted C₁₋₆ alkylene, wherein the C₁₋₆ alkylene is optionally substituted with one or more substituents independently selected from halogen, —OH, —CN, —NO₂, —NH₂, oxo, —C₁₋₁₀ haloalkyl, —O—C₁₋₁₀ alkyl, C₂₋₁₀ alkenyl, C₂₋₁₀ alkynyl;
L⁵ is selected from an (O—CH₂—CH₂—)_{q}—(NR³)ₛ;
L⁶ is selected from an optionally substituted C₁₋₆ alkylene, wherein the C₁₋₆ alkylene is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
L⁷ is selected from a C₅₋₆ carbocyclene;
R² is selected from an optionally substituted 5- to 6-membered heterocycle, wherein the 5- to 6-membered heterocycle is optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₂₋₁₀ alkenyl, C₂₋₁₀ alkynyl;
R³ is selected from hydrogen and C₁₋₆ alkyl;
m is selected from 0 and 1;
n is selected from 0 and 1;
p is selected from 0 and 1;
q is selected from 0 to 8; and
s is selected from 0 and 1.

129. The compound or salt of any one of claims 127 to 128, wherein the ligand is selected from an antibody or an antigen-binding fragment thereof.

130. The compound or salt of claim 129, wherein the ligand is selected from a group consisting of chimeric antibodies, humanized antibodies, and fully human antibodies.

131. The compound or salt of claim 129, wherein the antibody or antigen-binding fragment thereof binds an antigen selected from the group comprising or consisting of CD19, CD20, CD22, CD30, CD37, CD79b, HER2, and PSMA.

132. The compound or salt of claim 131, wherein the antibody or antigen-binding fragment thereof binds to HER2.

133. The compound or salt of claim 129, wherein the antibody or antigen-binding fragment thereof is selected from trastuzumab, brentuximab, loncastuximab, rosopatamab, rituximab, pinatuzumab, polatuzumab, and naratuximab.

134. The compound or salt of claim 133, wherein the antibody is trastuzumab.

135. The compound or salt of claim 133, wherein the ligand is UC-961.

136. A pharmaceutical composition comprising a compound or salt of any one of claim 100 to 135 and a pharmaceutically acceptable excipient.

137. The use of a compound or salt of any one of claims 100 to 135, or the pharmaceutical composition of claim 136, in the treatment of a disease or disorder.

138. The use of a compound or salt of any one of claims 100 to 135, or the pharmaceutical composition of claim 136, in the treatment of a tumor.

139. The use of a compound or salt of any one of claims 100 to 135, or the pharmaceutical composition of claim 136, in the treatment of a cancer.

140. A method of treating a subject with a disease or disorder, comprising administering to the subject in need thereof a compound or salt of any one of claims 100 to 135 or a pharmaceutical composition of claim 136.

141. A method of treating a subject with a tumor, comprising administering to the subject in need thereof a compound or salt of any one of claims 100 to 135 or a pharmaceutical composition of claim 136.

142. A method of treating a subject with a cancer, comprising administering to the subject in need thereof a compound or salt of any one of claims 100 to 135 or a pharmaceutical composition of claim 136.

143. The method of claim 139 or 142, wherein the cancer is selected from the group consisting of lung cancer, kidney cancer, urethral cancer, colorectal cancer, prostate cancer, glioblastoma multiforme, ovarian cancer, pancreatic, breast, melanoma, liver, bladder, stomach, and esophageal cancers.
